(19) United States
(12) Patent Application Publication
(10) Pub. No.: US 2020/0406254 A1
(43) Pub. Date: Dec. 31, 2020

(54) Q-MAX CARD-BASED ASSAY DEVICES AND METHODS

(71) Applicant: Essenlix Corporation, Monmouth Junction, NJ (US)

(72) Inventors: Stephen Y. Chou, Princeton, NJ (US); Wei Ding, East Windsor, NJ (US); Yufan Zhang, Monmouth Junction, NJ (US); Ji Qi, Hillsborough, NJ (US)

(73) Assignee: Essenlix Corporation, Monmouth Junction, NJ (US)

(21) Appl. No.: 16/484,409

(22) PCT Filed: Feb. 13, 2018
(86) PCT No.: PCT/US2018/018007

§ 371 Date: Aug. 7, 2019

Related U.S. Application Data
(60) Provisional application No. 62/456,612, filed on Feb. 8, 2017.
(60) Provisional application No. 62/456,488, filed on Feb. 8, 2017.
(60) Provisional application No. 62/456,504, filed on Feb. 8, 2017.
(60) Provisional application No. 62/457,133, filed on Feb. 9, 2017.
(60) Provisional application No. 62/457,103, filed on Feb. 9, 2017.
(60) Provisional application No. 62/456,988, filed on Feb. 9, 2017.
(60) Provisional application No. 62/460,062, filed on Feb. 16, 2017.

(51) Int. Cl. B01L 3/00; G01N 33/543

(52) U.S. Cl. CPC B01L 3/502707; B01L 3/502746; G01N 33/54366; B01L 2300/0636; B01L 2400/0406; B01L 2300/0816; B01L 2300/0819; B01L 2300/0851; B01L 2300/069

(57) ABSTRACT

Among other things, the present invention is related to devices and methods of performing biological and chemical assays, devices and methods of performing a biological and chemical extraction from a liquid, and performing assays, such as but not limited to immunoassays and nucleic acid assays.

### CROSS-REFERENCE TO RELATED APPLICATIONS

This application is a § 371 national stage application of International Application PCT/US2018/018007 filed on Feb. 13, 2018, which claims the benefit of priority to U.S. Provisional Patent Application No. 62/456,488, filed on Feb. 8, 2017, U.S. Provisional Patent Application No. 62/456,612, filed on Feb. 8, 2017, U.S. Provisional Patent Application No. 62/456,504, filed on Feb. 8, 2017, U.S. Provisional Patent Application No. 62/456,988, filed on Feb. 9, 2017, U.S. Provisional Patent Application No. 62/457,133, filed on Feb. 9, 2017, U.S. Provisional Patent Application No. 62/457,103, filed on Feb. 9, 2017, and U.S. Provisional Application No. 62/460,062, which was filed on Feb. 16, 2017, the contents of which are relied upon and incorporated herein by reference in their entirety. The entire disclosure of any publication or patent document mentioned herein is entirely incorporated by reference.

### FIELD

Among other things, the present invention is related to devices and methods of performing biological and chemical assays, devices and methods of performing a biological and chemical extraction from a liquid, and performing assays, such as but not limited to immunoassays and nucleic acid assays.

### BACKGROUND

In many bio/chemical testing processes (e.g., immunoassay, nucleotide assay, blood cell counting, etc.), chemical reactions, and other processes, there are needs for methods, kits, and systems that can accelerate the process (e.g., binding, mixing reagents, etc.), quantify the parameters (e.g., analyte concentration, sample volume, etc.), and do so with a small sample volume.

On the other hand, there are needs to separate component from a composite liquid sample, e.g., plasma separation. Conventionally, centrifugation is the most commonly used technique to separate component from a composite liquid sample based on the difference in the centrifugal forces. This method is laborious, requiring sophisticated equipment and professional handling. It is especially unsuitable for small volume of samples, which become more and more desired in point-of-care settings and personal health management where miniaturized testing equipment is being quickly developed and commercialized. Other existing arts in the field involve the use of microfluidic channels, eliminating the need of large volume of the sample. However, the manufacturing of microfluidic channels is technically challenging and hardly cost-effective. Some other arts take advantage of various filter media, mainly composed of porous materials (like filter paper) or glass fibers, in combination with the housing and supporting apparatus. This method is usually cost-effective and easy to handle, but often requires discharging or transferring of the filtering product for further analysis or processing.

### SUMMARY OF INVENTION

The following brief summary is not intended to include all features and aspects of the present invention.

The present invention relates to the methods, devices, and systems that make bio/chemical sensing (including, not limited to, immunoassay, nucleic assay, electrolyte analysis, etc.) much faster, much more sensitive, much less steps and easy to perform, much smaller amount of samples required, much more convenient to use, much less or no needs for professional assistance, and/or much lower cost, than many current sensing being used.

Particularly, the present invention is related to QMAX (“QMAX” (Q.: quantification; M. magnifying, A. adding reagents, X: acceleration), also known as “CROF” (compressed regulated open flow)) card-based assay devices and methods. More specifically, the present invention is related to compressed open flow assay methods, devices, kits, and systems for performing squeeze-wash, dilution calibration, component separation, and multi-plate sample analyses.

Improve Assay—Accurate Metering of a Sample Volume

One aspect of the invention is the methods and devices that make at least a portion of a small droplet of a liquid sample deposited on a plate become a thin film with a precisely controlled, predetermined, and uniform thickness over large area. The uniform thickness can be less than 1 um. Furthermore, the invention allows the same uniform thickness to be maintained for a long time period without suffering evaporation in an open surface.

Another aspect of the invention is the methods and devices that utilize the uniform thin sample thickness formed by the invention to determine the precise volume of a portion or entire of the sample without using any pipette or alike.

Improve Assay—A Efficient Way to Decrease Unspecific Binding

Another aspect of the invention is the methods and devices that perform squeeze/sponge wash with a QMAX device.

Improve Assay—Easy Calibration of Dilution Factors

Another aspect of the invention is the methods that use a QMAX card to conveniently calibrate dilution factors of any sample, e.g., blood or plasma.

Component Separation with a QMAX Device

Yet another aspect of the invention is the methods and devices that use a QMAX card to separate certain component from a composite liquid sample and obtain the liquid sample without the component therein and/or extract the component from the sample.

### BRIEF DESCRIPTION OF THE DRAWINGS

The skilled artisan will understand that the drawings, described below, are for illustration purposes only. The drawings are not intended to limit the scope of the present teachings in any way. The drawings are not entirely in scale. In the figures that present experimental data points, the lines that connect the data points are for guiding a viewing of the data only and have no other means.

FIG. 1 is a schematic representation of an example of an assay method according to the present disclosure.

FIG. 2 is a schematic representation of an assay plate according to the present disclosure.

FIG. 3 is a schematic representation of a second plate according to the present disclosure.

FIG. 4 is a schematic representation of a wash pad according to the present disclosure.

FIG. 5 is a schematic representation of a sample and an assay plate.

FIG. 6 is a schematic representation of an assay assembly (exploded diagram).

FIG. 7 is a schematic representation of an assay assembly being squeezed.

FIG. 8 is a schematic representation of a wash pad used with an assay plate.

FIG. 9 is a chart comparing results of assays performed with various techniques. “No wash” is an assay without a wash step. “Sponge wash” is the same assay performed with a squeeze wash according to the present disclosure. “Normal wash” is the same assay performed with a conventional wash step. Assay and wash parameters are given in Table 1.

FIG. 10 is a schematic representation of a kit and kit components according to the present disclosure.

FIG. 11 is a schematic side view of a wash pad.

FIG. 12 is a flow diagram of an exemplary embodiment of a method of determining the dilution factor for a sample provided by the present invention.

FIG. 13 is a flow diagram of another exemplary embodiment of a method of determining the dilution factor for a sample provided by the present invention.

FIG. 14 shows an embodiment of a QMAX device.

FIG. 15 is a flow diagram of an exemplary embodiment of a method to determine the dilution factor for a blood sample, according to the present invention.

FIG. 16 shows representative images of undiluted (a) and 10× diluted (b) samples obtained in bright field mode.

FIG. 17 shows schematically exemplary embodiments of the device and method for separating component from a composite liquid sample as provided by the present invention.

FIG. 18 is a flow chart for an exemplary embodiment of the method disclosed in the present invention.

FIG. 19 shows the representative images of the filtering products resulted from different experimental configurations of the device when used for plasma separation.

FIG. 20 shows the results of a triglyceride (TG) assay using the filtering products from the experimental filtering device as the assay sample and the QMAX device as the assay device.

FIG. 21 shows an embodiment of a QMAX (Q: quantification; M: magnifying, A. adding reagents, X: acceleration; also known as compressed regulated open flow (CROF)) device, which comprises a first plate, a second plate and a third plate. Panel (A) shows the perspective view of the plates in an open configuration when the plates are separated apart, panel (B) shows the sectional view of the plates at the open configuration.

FIG. 22 shows an exemplary embodiment of the QMAX device and the process to utilize the QMAX device to filter and analyze a liquid sample. Panel (A) shows the sectional view of a QMAX device in an open configuration, where sample is deposited on the filter, which is placed on top of the first plate, panel (B) shows the sectional view of a QMAX device when the third plate is pressed on top of the filter, pushing part of the sample to flow through the filter, panel (C) shows a sectional view of the QMAX device when the third plate 30 is opened after filtering and before the second plate is pivoting towards the first plate, panel (D) shows a sectional view of the QMAX device in a closed configuration when the part of the sample that flows through the filter is pressed into a layer of uniform thickness.

FIG. 23 shows an exemplary embodiment of the QMAX device. Panel (A) shows the top view of a QMAX device that comprises notches in the closed configuration; panel (B) shows the top view of a QMAX device that comprises notches in the closed configuration when the filter is placed on top of the first plate.

### DETAILED DESCRIPTION OF EXEMPLARY EMBODIMENTS

The following detailed description illustrates some embodiments of the invention by way of example and not by way of limitation. The section headings and any subtitles used herein are for organizational purposes only and are not to be construed as limiting the subject matter described in any way. The contents under a section heading and/or subtitle are not limited to the section heading and/or subtitle, but apply to the entire description of the present invention.

The citation of any publication is for its disclosure prior to the filing date and should not be construed as an admission that the present claims are not entitled to antedate such publication by virtue of prior invention. Further, the dates of publication provided can be different from the actual publication dates which can need to be independently confirmed.

### Definitions

The following definitions are set forth to illustrate and describe the meaning and scope of (a) certain embodiments of the invention and (b) certain terms used in the section of “Detailed Description of Exemplary Embodiments.”

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

Any patents, patent applications, or other references that are incorporated by reference herein and (1) define a term in a manner that is inconsistent with and/or (2) are otherwise inconsistent with, either the non-incorporated portion of the present disclosure or any of the other incorporated references, the non-incorporated portion of the present disclosure shall control, and the term or incorporated disclosure therein shall only control with respect to the reference in which the term is defined and/or the incorporated disclosure was present originally.

The terms used in describing the devices, systems, and methods herein disclosed are defined in the current application, or in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, all of which applications are incorporated herein in their entireties for all purposes.

“QMAX” (Q.: quantification; M: magnifying, A. adding reagents, X: acceleration; also termed as self-calibrated compressed open flow (SCOF)) devices, assays, methods, kits, and systems are described in: U.S. Provisional Patent Application No. 62/202,989, which was filed on Aug. 10, 2015, U.S. Provisional Patent Application No. 62/218,455, which was filed on Sep. 14, 2015, U.S. Provisional Patent Application No. 62/293,188, which was filed on Feb. 9, 2016, U.S. Provisional Patent Application No. 62/305,123, which was filed on Mar. 8, 2016, U.S. Provisional Patent Application No. 62/369,181, which was filed on Jul. 31, 2016, U.S. Provisional Patent Application No. 62/394,753, which was filed on Sep. 15, 2016, PCT Application (designating U.S.) No. PCT/US2016/045437, which was filed on Aug. 10, 2016, PCT Application (designating U.S.) No. PCT/US2016/051775, which was filed on Sep. 14, 2016, PCT Application (designating U.S.) No. PCT/US2016/051794, which was filed on Sep. 15, 2016, and PCT Application (designating U.S.) No. PCT/US2016/054025, which was filed on Sep. 27, 2016, all of these disclosures are hereby incorporated by reference for their entirety and for all purposes.

The terms “CROF Card (or card)”, “COF Card”, “QMAX-Card”, “Q-Card”, “CROF device”, “COF device”, “QMAX-device”, “CROF plates”, “COF plates”, and “QMAX-plates” are interchangeable, except that in some embodiments, the COF card does not comprise spacers, and the terms refer to a device that comprises a first plate and a second plate that are movable relative to each other into different configurations (including an open configuration and a closed configuration), and that comprises spacers (except some embodiments of the COF) that regulate the spacing between the plates. The term “X-plate” refers to one of the two plates in a CROF card, wherein the spacers are fixed to this plate. More descriptions of the COF Card, CROF Card, and X-plate are described in the provisional application Ser. Nos. 62/456,065, filed on Feb. 7, 2017 and U.S. Provisional Application No. 62/456,287, which was filed on Feb. 8, 2017, all of which is incorporated herein in their entirety for all purposes.

### 1 Squeeze/Sponge-Wash Assay Methods, Kits, and Systems

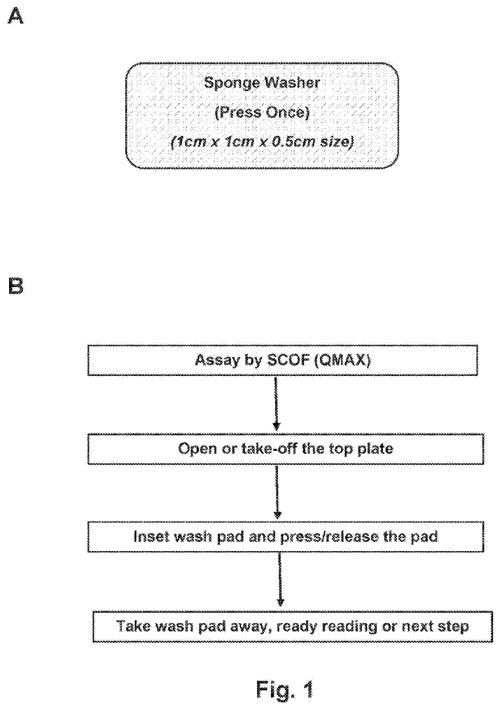

FIGS. 1-11 illustrate squeeze-wash self-calibrated compressed open flow assay methods, kits, and systems. In general, in the drawings, elements that are optional or alternatives are illustrated in dashed lines. However, elements that are illustrated in solid lines are not essential to all embodiments of the present disclosure, and an element shown in solid lines is omitted from a particular embodiment without departing from the scope of the present disclosure. Elements that serve a similar, or at least substantially similar, purpose are labeled with numbers consistent among the figures. Like numbers in each of the figures, not all the corresponding elements are discussed in detail herein with reference to each of the figures. Similarly, not all elements are labeled or shown in each of the figures, but reference numerals associated therewith are used for consistency. Elements, components, and/or features that are discussed with reference to one or more of the figures are included in and/or used with any of the figures without departing from the scope of the present disclosure.

Generally, drawing elements are referenced according to the following table.

| Number | Brief description |
|---|---|
| 10 | Assay assembly-assembly of sample 50 and one or more plates 20 and |
| | optionally wash pad 40 |
| 12 | Kit - assay kit including an assay plate 22, a Second plate 24, and a wash pad 40 |
| 20 | Plate-base structure for assay assembly and assay kit. A plate can also be called a |
| | substrate, a cover, a film. Plates are rigid or flexible. Plates generally are thin relative |
| | to their lateral dimensions. Plates generally have a relatively smooth and flat surface |
| | when neglecting the presence of optional spacers 70. Plates generally are configured |
| | for optical detection, with one or both being optically transparent. |
| 22 | Assay plate - a type of plate 20. Assay plates include one or more assay sites 30. |
| | Some assay plates are referred to as first plates. |
| 24 | Second plate - a type of plate 20 used in conjunction with the assay plate 22. Some |
| | second plates are also referred to as cover plates and/or reagent plates (especially |
| | when the second plate includes reagent 60). In some embodiments, the Second plate is |
| | also an assay plate 22. |
| 26 | Receiving plate - a type of plate 20 that receives a sample 50 and/or that is configured |
| | to receive sample 50. Either the assay plate 22 or the second plate 24 |
| | (or both) is a receiving plate. |
| 28 | Assay surface - the working surface of a plate 20. in some embodiments, assay |
| | surfaces include assay sites 30, reagents 60, and spacers 70. |
| 30 | Assay site - an assay region of the assay surface 28 of the assay plate 22. Assay |
| | sites include assay components such as capture agent 54 to perform an assay localized |
| | at the assay site. Some assay sites are also referred to as measurement |
| | sites. |
| 40 | Wash pad - a pad of porous media 42 configured to hold wash solution 44. Wash |
| | pads are configured to expel wash solution 44 when squeezed (compressed) and |
| | are configured to draw in fluids when squeezing is stopped. In some |
| | embodiments, wash pads are referred to as sponges, sponge washers, and/or |
| | washing sheets. |
| | Porous media-absorbent media with an open volume that can be reduced when |
| | squeezed (compressed). Generally, porous media is resilient and substantially |
| | returns to its uncompressed state and shape when squeezing (compression) is |
| | stopped. |
| 44 | Wash solution - a liquid solution configured to carry unbound assay components |
| | away from the assay site 30. Wash solution generally includes water, buffer, and/or |
| | solvent. |
| 50 | Sample - an assay sample that is to be tested for the presence and/or activity of analyte |
| | (analyte molecules 52). Samples generally are biological samples and in some |
| | embodiments are direct samples from a subject (with or without dilution and/or |
| | suspension) such as cells, tissues, bodily fluids, stool, hair, etc. |
| 52 | Analyte molecule - an individual analyte entity, the Subject of the assay. As used |
| | herein, the analyte molecule is the analyte entity, regardless of whether the entity is a |
| | molecule, an atom, a complex, a particle, etc. Analyte types include proteins, |
| | peptides, DNA, RNA, nucleic acid, small molecules, cells (including blood cells, |
| | platelets), cells, issues, viruses, and nanoparticles. |
| 54 | Capture agent - an assay component that binds to a target analyte (analyte |
| | molecule 52) through a specific interaction, generally with high affinity (e.g., with a |
| | dissociation constant (KD) less than 10M (molar)). Generally, capture agents do not |
| | significantly bind other components of the sample 50. Examples of capture |
| | agents include antibodies, proteins, and nucleic acids. |
| 56 | Blocking agent - an optional assay component that reduces off-target binding |
| | (binding of components other than the analyte), non-specific binding (undesired |
| | binding of the analyte or other assay components), and/or other types of assay |
| | interference. In some embodiments, blocking agents are included at the assay site(s) |
| | 30, the assay surface 28 to off-target binding, and/or in solution. |
| 58 | Linker- an optional assay component that specifically binds the capture agent 54 to |
| | the assay site 30. For example, in certain embodiments the linker is Protein A, a |
| | protein that specifically binds to immunoglobulins of certain species. |
| 60 | Reagent - an assay component, e.g., the capture agent 54, the detection agent 62, |
| | a cofactor, a lysing agent, etc. Reagents are added to the assay in dry or fluid |
| | form. For example, one or more reagents are dried on the assay surface 28 (e.g., |
| | at assay site 30) of a plate 20. As another example, reagents are added to the |
| | sample by liquid addition before, after, or during contact with one or both plates 20. |
| 62 | Detection agent- an assay component that binds to the target analyte (analyte |
| | molecule 52) and/or the target analyte molecule when bound to the capture agent |
| | 54. Additionally or alternatively, the detection agent is a substrate or chemical |
| | reactant acted upon by the target analyte bound to the capture agent. The detection |
| | agent is an antibody that recognizes a site on the analyte that is different from the |
| | capture agent's binding site. Generally, the detection agent binds with high affinity |
| | (e.g., KD < 10M) to the analyte and/or the capture agent-analyte complex. Examples |
| | of detections agents include antibodies, proteins, and nucleic acids. In some |
| | embodiments, the detection agents include a label64 and/or is selected and/or adapted |
| | to bind to a label 64. |
| 64 | Label - a detectable moiety Such as an enzyme, a fluorophore, a luminophore |
| | (chemiluminescent, electrochemiluminescent), a radioisotope, a mass label, etc. |
| | Labels are generally optically detectable and are acoustically and/or electrically |
| | detectable. |
| 70 | spacers - structures that regulate the squeezed thickness between plates 20. |
| | spacers are surface structure or bound to one or both assay surfaces 28 of the assay |
| | plate 22. Additionally, or alternatively, the sample 50 include spacers. |
| | spacers are beads or other particulate, generally with a narrow size distribution such |
| | that the regulated spacing between plates 20 is substantially characterized by the |
| | average size of the spacers. In some embodiments, spacers are embossed, etched, or |
| | otherwise formed on an assay surface 28 and/or within an assay site 30. Bound and/or |
| | integral spacers have a substantially uniform height that characterizes the regulated |
| | spacing between plates 20. |
| 110 | Sample alignment mark - a mark on the receiving plate 26 that facilitates placement of |
| | the sample 50 on the receiving plate 26. Sample alignment marks are on the assay |
| | surface 28, within the material of the receiving plate 26, and/or on the surface |
| | opposite the assay surface 28. In some embodiments, sample alignment marks |
| | indicate the assay site 30 but do not generally obscure the assay site 30. |
| 112 | Plate alignment fiducial - a mark or structure on one or both of the assay plate 22 and |
| | the second plate 24. Plate alignment fiducials facilitate placement of the assay |
| | plate 22 and the second plate 24 together. In some embodiments, plate alignment |
| | fiducials are edges or marks that are aligned when placing the plates together. In |
| | some embodiments, plate alignment fiducials include a shoulder, a pin, a socket, etc. |
| | that mates to a corresponding structure on the opposite plate. In some |
| | embodiments, plate alignment fiducials are configured to assist plate alignment by |
| | hand or by machine. |
| 116 | Tab (plate) - a projection, grip, or handle of a plate 20 that is configured to facilitate |
| | handling of the plate and/or separation of the plates. In some embodiments, tabs 116 is |
| | extensions of the plate body (in the general plane of the plate). |
| 140 | Backing - an optional component of the wash pad 40 that is configured for ease of |
| | handling and/or to assist with squeezing the wash pad. |
| 142 | Tab (wash pad) - a projection, grip, or handle of a wash pad 40, generally a |
| | component of the backing 140. Tabs 142 are configured to facilitate handling of |
| | the wash pad 40, separating the wash pad from the assay plate 22, loading the |
| | wash pad with wash solution 44, and/or removing the wash pad seal 146. |
| 144 | Wash surface - a surface of the porous media 42 of a wash pad 40 that is |
| | configured for contact with the sample 50 and/or the assay plate 22 during an |
| | assay. The wash surface generally is opposite to the backing 140 (i.e., one side of |
| | the wash pad is the wash surface and the other side is the backing). |
| 146 | Wash pad seal - a liquid barrier that contain and/or seal wash solution 44 in the |
| | porous media 42 of a wash pad 40. In some embodiments, the wash pad seal is |
| | an impervious film or membrane encasing the porous media 42 (or the porous |
| | media not covered by the backing 140). In some embodiments, the wash pad seal is |
| | used to seal the wash pad 40 loaded with wash solution 44 until the time of use: |
| | of the wash pad to Wash the assay plate 22. |

The squeeze-wash or sponge wash technology (also referred to as S-Technology) can be used in QMAX (Q: quantification; M: magnifying, A. adding reagents, X: acceleration; also termed SCOF: self-calibrated compressed open flow) assays. Specifically, the squeeze-washing technology is used to reducing non-specific binding and improve the specificity of the assay. It should also be noted the squeeze-washing technology can also be used in other assays besides the QMAX assays. In the QMAX assay, a sample containing analytes is squeezed between two plates. At least one of the plates or the sample has spacers that are configured to regulate the sample thickness when squeezed between the plates. The squeezing causes the sample to spread between the plates and limits diffusion to less than unconstrained, three-dimensional diffusion (three-dimensional Brownian motion). In some embodiments, the squeezed thickness is small enough that diffusion is substantially two-dimensional. The limited thickness improves (accelerates) reagent incubation time for reagents traversing the thickness (reagents mix across the thickness relatively rapidly). The constrained lateral diffusion isolates assay sites along the plate surface (reagents mix laterally (transverse to the thickness) relatively slowly).

Many assays are adapted to the self-calibrated compressed open flow technique. Some assays benefit from, or require, a wash step. Assay wash steps typically are designed to remove unbound assay components and reduce off-target binding. Conventional washing techniques include rinsing (allowing excess solution to drain away), dunking, and cycles of aspiration and dispensing. In the self-calibrated compressed open flow technique, some of the benefits of the increased assay speed and efficiency could be lost by conventional washing.

In some embodiments of the sponge washing technology, any of the following are implemented or described:

(1) A sponge sheet (or any porous and absorbent material) is used with a wash solution (e.g. water) to ash an assay surface.
(2) The sponge is a flexible porous material; its pore size can be reduced under a compression pressure and return to the original size when the pressure is removed.
(3) when a sponge sheet covers an assay surface, a pressing of the sponge makes the washing solution in the sponge touch and wash the assay surface. Then a release of the pressure makes the waste washing solution reabsorbed back to the sponge, leaving the assay surface washed and nearly free of waste washing solution.
(4) The assay washed in this way is ready for a next step, such as reading or subsequent reagent interaction.
(5) The S-technology wash can be used repeatedly, if necessary.
(6) FIG. 1, panel (A), provides an example of the sponge, which as a 1 cm×1 cm 0.5 cm size.
(7) The sponge can have a plastic back plane for easy handling and to facilitate a washing.

As shown in FIG. 1, panel (B), in the squeeze-wash self-calibrated compressed open flow technique, the plates are separated (e.g., opened) after the self-calibrated compressed open flow squeezing step. This initial squeezing step causes assay components to mix and/or react and causes at least some assay components to bind to at least one of the plate surfaces. Washing is performed by separating the plates and by contacting the assay site (the site with bound analyte) with a wash pad loaded with wash solution. In some embodiments, the wash pad is preloaded with wash solution; the wash pad is loaded (filled) with wash solution just before contacting the assay site, and/or the Wash pad is loaded after contacting the assay site. Washing continues by squeezing the wash pad on the assay site. Squeezing the loaded wash pad causes wash solution to be expelled from the wash pad and contact/rinse the assay site. In some embodiments, the washing procedure includes releasing the force that squeezes the wash pad, in which case, the wash pad expands to its original shape and draws in neighboring fluids (e.g., wash solution mixed with unbound assay components). In certain embodiments, the used wash pad is removed from the assay site to prepare the plate for subsequent measurements or assay steps (such as further assay additions and/or washings with different reagents). Additionally or alternatively, a wash pad is reused in place (e.g., by reloading with wash solution and re-squeezing). The dimensions of the wash pad indicated in panel (A) of FIG. 1 (e.g., 1 cm×1 cm×0.5 cm) are illustrative only and do not represent a limitation or bound on the size.

In some embodiments, the wash pad is squeezed by one of the plates 20. In some embodiments, the wash pad is squeezed with an object that this is not part of the assay assembly. In certain embodiments, the wash pad is squeezed with a human hand.

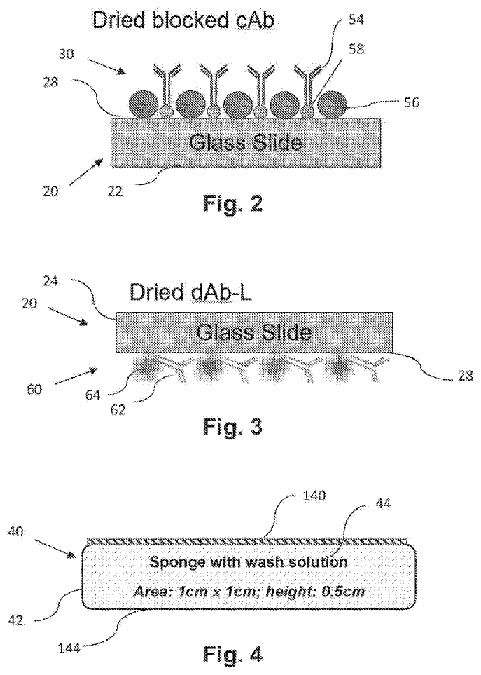

FIG. 2 illustrates an assay plate 22 (also referred to as a first plate). The assay plate 22 includes an assay surface 28 and an assay site 30 on the assay surface. The assay site 30 has bound capture agents 54. The capture agents 54 are schematically illustrated as antibodies though capture agents are not required to be antibodies. In some embodiments, the assay site 30 includes blocking agent 56 to reduce non-specific and off-target binding at the assay site. In some embodiments, the capture agents 54 is bound to the assay site 30 by linkers 58 (e.g., Protein A, avidin, etc.). Additionally or alternatively, the capture agents 54 are covalently bound (directly or via linkers 56) to the assay surface 28 at the assay site 30. The capture agents 54 are bound to the assay site 30 in dried and/or environmentally stabilized form. In some embodiments, the capture agent 54 and/or the blocking agent 56 are dried and/or coated on the assay site 30 of the first plate 22.

In some embodiments, the assay plate 22 includes a plurality of assay sites 30. Each assay site 30 includes the same or different types of capture agents 54. For example, each assay site 30 has a different type of capture agent 54 to perform an assay for a different type of analyte, or each assay site 30 has the same type of capture agent 54 but in different concentrations. As another example, an assay plate 22 includes one or more replicate assay sites (e.g., duplicates), with each assay site 30 of the replicate assay sites having the same type of capture agent 54 to perform the same assay.

FIG. 3 illustrates a second plate 24. In the example of FIG. 3, the second plate 22 includes reagent 60 on the assay surface 28. The reagent 60 in this example is detection agents 62. In some embodiments, the detection agents 62 include a label 64 and are referred to as labeled detection agents. The detection agents 62 are schematically illustrated as antibodies though detection agents are not required to be antibodies. The detection agents 62 are associated, adhered, and/or bound to the assay surface 28. Generally, detection agents 62 are placed on the assay surface 28 in a form that permits the detection agents to dissociate from the assay 10 surface and diffuse to the assay site 30 of the assay plate 22. In some embodiments, detection agents 62 are dried onto the assay surface 28 and are in dried and/or environmentally stabilized form.

Referring to FIGS. 2-3, the assay plate 22 and the second plate 24 are components of the plate combination 20. In some embodiments, the assay plate 22, the second plate 24, or both plates comprise spacers that are fixed on the respective surface(s) of the plate(s). When the plates are pressed together, with the assay surfaces facing each other, the spacers control the spacing between the plates 20. In addition, if the plates 20 are pressed after the deposition of the sample, the spacers control the thickness of the sample, forming a thin and uniform thickness.

FIG. 4 illustrates a wash pad 40. The wash pad 40 includes porous media 42 and, at least when prepared for use, includes wash solution 44. The wash pad 40 is configured, selected, and/or adapted to hold (retain) wash solution 44 in an uncompressed state and to expel at least some of the wash solution upon compression. As also shown in FIGS. 8, 10, and 11, in some embodiments the wash pad includes a backing 140 and/or a tab (not shown). The wash pad 40 has a wash surface 144 configured to contact the assay surface 28 and/or the assay site 30 of the assay plate 22.

The porous media 42 of the wash pad 40 is absorbent and includes, and/or is, a foam (reticulated and/or open cell), a fibrous material, a gel, a sponge, etc. Examples of materials include cellulose, polyester, polyurethane, gelatin, agarose, polyvinyl alcohol, and combinations thereof. Generally, the porous media 42 is selected and/or configured to avoid specific binding of the analyte molecules 52, the sample 50, and/or assay reagents 60. However, in some embodiments, the porous media 42 is selected and/or configured to preferentially and/or specifically bind certain assay components (e.g., components of the sample 50).

In some embodiments, the wash pad 40 includes a backing 140 for ease of handling and/or to assist with squeezing. In certain embodiments, the backing 140 includes, and/or is, a non-absorbent layer and/or a water impermeable layer. In certain embodiments, the backing 140 is rigid and/or resilient. Generally, the porous media 42 is bonded or otherwise attached to the backing 140 with the wash surface 144 of the porous media facing away from the backing (i.e., one side of the wash pad is the backing and the other side includes the wash surface). In certain embodiments, the backing 140 (and/or the wash pad 40 generally) includes a tab (not shown) to aid in handling the wash pad 40 and/or to aid in separating the wash pad from the assay plate 22.

The porous media 42 and the pores in the porous media are configured to hold wash solution 44. Generally, the porous media 42 has a substantial open volume, e.g., greater than 50%, greater than 80%, or greater than 90% open, that holds the wash solution 44. Typically, the average effective pore diameter is about 0.1 um to about 1,000 um so that capillary forces retain wash solution 44 within the pores. The porous media 42 is configured to reduce the open volume when subject to squeezing (compressive force). When previous loaded with wash solution 44, the reduced volume due to squeezing causes at least some of the wash solution 44 to be expelled from the wash pad 40. Additionally, when a previously compressed (squeezed) wash pad 44 is released from compression, the wash pad relaxes back to substantially its original shape, causing the pores to expand and the open volume to increase. This action draws fluids into the wash pad 44 when the compressive force is released.

When used as described herein, the squeezing of the wash pad 40 causes wash solution 44 to rinse the assay surface 28 and/or the assay site 30 of the assay plate 22. When used as described herein, the release of the squeezing of the wash pad 40 causes rinsed solution (the wash solution 44 and the unbound sample 50) to be substantially drawn into the wash pad 40.

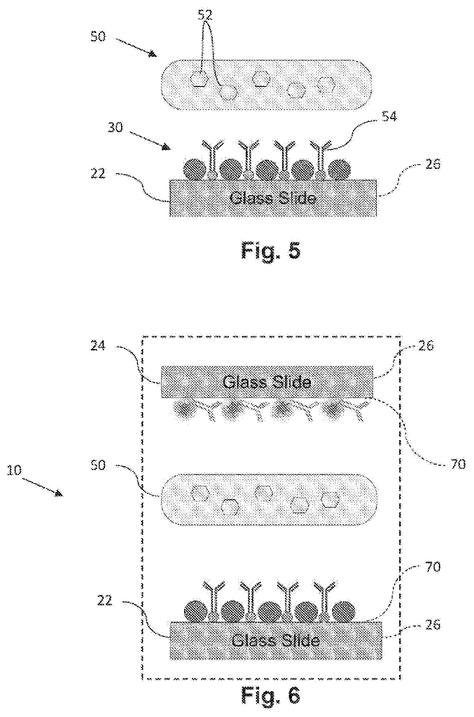

FIG. 5 illustrates a sample 50 in context with an assay plate 22. The sample 50 generally includes one or more species of analytes, with each species of analyte found as analyte molecules 52. As the assay is configured to detect the presence, quantity and/or activity of analyte species, certain samples 50 has little to no analyte molecules 52 (or no analyte molecules of a particular analyte species). In some embodiments, the sample 50 is placed in contact with the assay site 30. In some embodiments, the sample 50 is placed on the assay plate 22 on or near the assay site 30. Additionally or alternatively, the sample 50 is placed on the second plate 24 in a location that will be over or near the assay site 30 when the plates 20 are placed together. In some embodiments, the sample 50 is drawn to a location at or near the assay site 30 by capillary action of the sample between the plates 20. For example, the plates 20 are spaced apart by a spacing sufficient to permit capillary action of the sample 50, and the sample 50 is introduced to the plates 20 at an open edge of the spaced-apart plates 20. As indicated in the descriptions above, in some embodiments capture agents (e.g. antibodies) and/or blocking agents are dried and coated on the assay site of the plates 20.

The plates 20 are moveable relative to each other into different configurations. One of the configuration is an open configuration, in which the two plates 20 are partially or completely separated apart, the spacing between the plates is not regulated by the spacers, allowing a liquid sample to be deposited on one or both of the plates. Another of the configurations is a closed configuration which is configured after the sample deposition in the open configuration, and in the closed configuration: at least part of spacing between the two plates 20 is regulated by the plates and the spacers and at least part of the sample is compressed into a layer of uniform thickness, which is in contact with the capture agent.

As shown in FIG. 6, the assay surface 28 of each plate 20 is the operative surface of the plate. The sample 50 contacts the assay surfaces 28. Generally, when assembled in the assay assembly 10, the sample 50 is sandwiched between the plates 20 with the assay surfaces 28 of the respective plates 20 facing each other. In some embodiments, the assay plate 22 and the second plate 24 are connected by turning structures such as one or more hinges, which allow the plates 20 to pivot against one another. The plates 20 connected by structures such as hinges are termed a QMAX card.

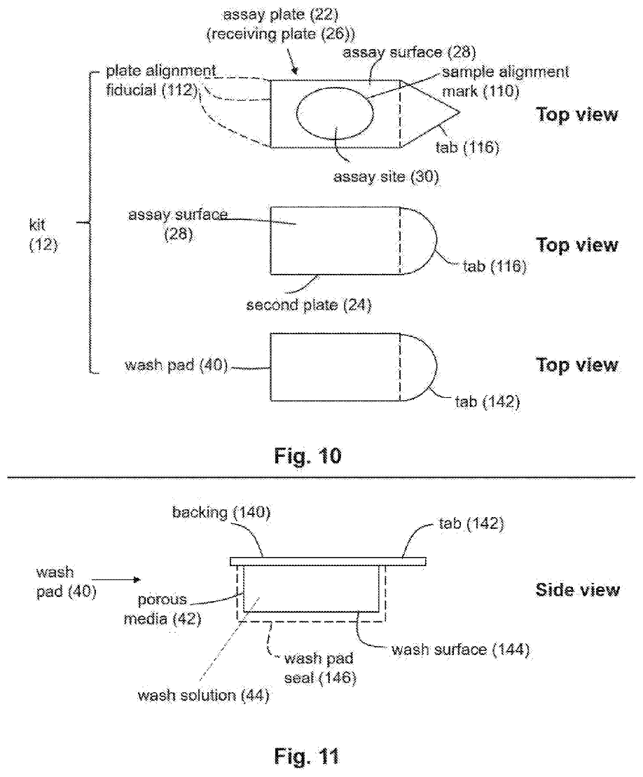

When the plates 20 are assembled in the assay assembly 10, generally no precise alignment is needed. The sample 50 between the plates 20 is squeezed by the pressing of the plates, causing the sample to expand laterally across the assay surfaces 28. This extension of the sample 50 as it is squeezed permits the sample to be placed with coarse precision on the assay surface 28 of the receiving plate 26 and permits the plates 20 to be contacted together with coarse precision. The extension of the sample 50 will substantially fill the assay site(s) 30 on the assay plate 22 even if the sample was not initially aligned with the assay site(s) 30. In some embodiments, the receiving plate 26 includes a sample alignment mark 110 to guide placement of the sample 50. In some embodiments, one or more of the plates 20 include plate alignment fiducials 112 (e.g., a mark or a physical structure as shown in FIG. 10) to guide placement of the plates together. For example, the plates 20 has one or more edges that are aligned when the plates are sufficiently aligned.

As indicated in FIG. 6, one or more of the assay surface 28 of the assay plate 22, the assay surface 28 of the second plate 24, and the sample 50 generally includes spacers 70 (not shown). The spacers are configured, sized, selected, and/or adapted to define a minimum distance (also referred to as a regulated distance and/or a threshold thickness) between the assay plate 22 and the second plate 24. In some embodiments, the minimum distance is a non-zero distance and is the same as the height of the spacers. In certain embodiments, the minimum distance between the plates 20 is also the same as the thickness of the sample 50 when the plates are pressed together, rendering the sample 50 into a thin layer. The distance is minimum distance between the plates 20 in the local neighborhood. In some embodiments, individual spacer 70 contacts both plates 20 (e.g., a spacer is integral with one plate and contact the other plate when the plates are squeezed together). Generally, the height (length of the dimension between the plates) of the spacers 70 determines the minimum distance. In some embodiments, the minimum distance is the height of the spacers 70 plus a residual height of the sample between the spacer and the plate(s). The minimum distance, the height of the spacers, and/or the thickness of the sample 50, generally is 3 nm or less, 10 nm or less, 50 nm or less, 100 nm or less, 200 nm or less, 500 nm or less, 800 nm or less, 1000 nm or less, 1 μm or less, 2 μm or less, 3 μm or less, 5 μm or less, 10 μm or less, 20 μm or less, 30 μm or less, 50 μm or less, 100 μm or less, 150 μm or less, 200 μm or less, 300 μm or less, 500 μm or less, 800 μm or less, 1 mm or less, 2 mm or less, 4 mm or less, or in a range between any two of the values.

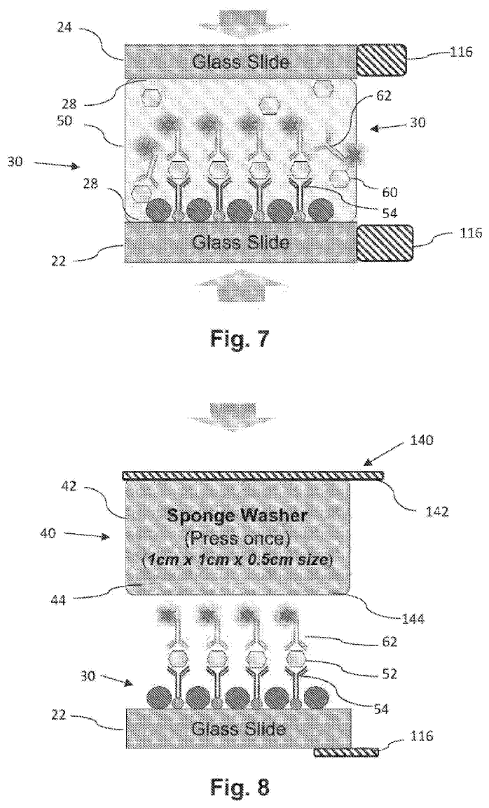

FIG. 7 illustrates an assay assembly 10 when squeezed into a closed configuration. The assay plate 22 and the second plate 24 are squeezed together with the sample 50 between the plates. The sample 50 contacts the assay site 30 (rehydrating the assay site and/or the capture agents 54 if needed). The sample 50 also contacts the assay surface 28 of the second plate 24, permitting reagents 60 (such as detection agents 62 as shown) to mix in the sample and migrate to the assay site 30. The contact of the sample 50 with the reagents 60 on the assay surface 28 of the second plate 24 releases the reagents from the assay surface and rehydrates and/or dissolves the reagents.

In the closed configuration (squeezed condition), as illustrated in FIG. 7, the assay assembly 10 is incubated to permit the capture agents 54, the sample 50, the analyte molecules 52, the detection agents 62, and/or other reagents 60 to mix and/or react. Due to the reduced thickness of the sample 50 between the plates (the distance regulated by the spacers 70), the time for a molecule or other assay component to diffuse along the thickness is greatly reduced as compared to the original sample thickness. A sample thickness of less than about 200 um strongly impacts the molecular diffusion. A sample thickness of less than about 20 um constrains diffusion to substantially two dimensions (motion in the thickness direction is more ballistic than diffusive). Incubation time can be substantially reduced from the incubation time required when performing a similar assay in a bulk format (e.g., in a multiwell plate). The useful incubation time in the squeeze-wash QMAX assay format is less than 500 seconds, less than 100 seconds, less than 50 seconds, less than 20 seconds, less than 5 seconds, or less than 2 seconds, or in a range between any of the two values. Relative to the time for hand manipulation of plates 20, the useful incubation time is essentially instantaneous. The assay assembly 10 is held in the squeezed condition for a period of time longer than necessary to cause the assay components to mix and react.

FIG. 8 illustrates the wash pad 40 used to wash the assay plate 22. The wash pad 40 is placed in contact with the assay surface 28 and/or the assay site 30. The wash pad 40 is preloaded with wash solution 44 and/or wash solution 44 is added to the wash pad 40. The wash pad 40 and the assay plate 22 are squeezed together to expel wash solution 44 from the wash pad onto the assay plate 22. In some embodiments, the squeezing of the wash pad 40 is facilitated by the optional backing 140 and/or the second plate 24. In certain embodiments, the second plate 24 is used to press the wash pad 40. In some embodiments, the assay assembly 10 comprises one or more hinges that connects the assay plate 22 and the second plate 24, the plates 20 pivot against each other, switching between open and closed configurations. In certain embodiments, after incubation in the closed configuration, the second plate 24 is opened and the wash pad 40 is placed against the assay surface on the assay plate, then the Second plate 24 is pressed against the Wash pad 40, depositing the wash solution 44 on the assay plate 22 to wash the assay site, with the release of the second plate 24, the wash solution 44 is reabsorbed into the wash pad 40.

After the incubation and switching to the open configuration, the wash pad is placed on the assay plate 22 so that the wash pad 40 contacts the assay plate 22 (the plate 20 with the Capture agents 54 and the assay site(s) 30), generally without any need for precise alignment. The wash pad 40 generally is sized larger than the area covered by all the relevant assay sites 30. For example, in some embodiments the wash pad 40 has a lateral size substantially the same as the size of the assay surface 28 of the assay plate 22. Additionally, the wash pad 40 is sized to hold sufficient wash solution 44 to rinse the relevant assay sites 30. Hence, when the wash pad 40 is squeezed, excess wash solution 44 flows beyond the periphery of the wash pad.

In some preferred embodiments, there are spacers (also termed “wash spacers”) between the wash surface 144 of the wash pad 40 and the assay plate 22 that are configured to maintain the non-zero spacing between the wash surface 144 and the assay site 30, in order to prevent the direct contact therebetween during squeezing and thereby the potential physical removal by the direct contact of the reagent 60 (e.g., the capture agent 54, the detection agent 62) and/or the analyte 52 bound therewith in the relevant assay site 30. In some embodiments, wash spacers are part of the spacers 70 of the assay plate 22 and are within and/or adjacent to the assay site 30. Additionally or alternatively, said spacers are part of the spacers 70 of the sample 50 and, following the separation of the assay plate 22 and the second plate 24 after the assay, are located within and/or adjacent to the assay site 30. Additionally or alternatively, wash spacers are part of the wash surface 144 of the wash pad 40 (termed “wash pad spacers”), and following the contact between the wash pad 40 and the assay plate 22, are within and/or adjacent to the assay site 30.

In these preferred embodiments, the wash surface 144 is configured (e.g. rigid enough) to, combined with the wash spacers, prevent the direct contact with the assay site 30 during squeezing, whereas the Wash pad 40 in its entirety, as described above, is configured, selected, and/or adapted to hold (retain) wash solution 44 in an uncompressed state and to expel at least some of the wash solution upon compression.

### 1.3 Experiments

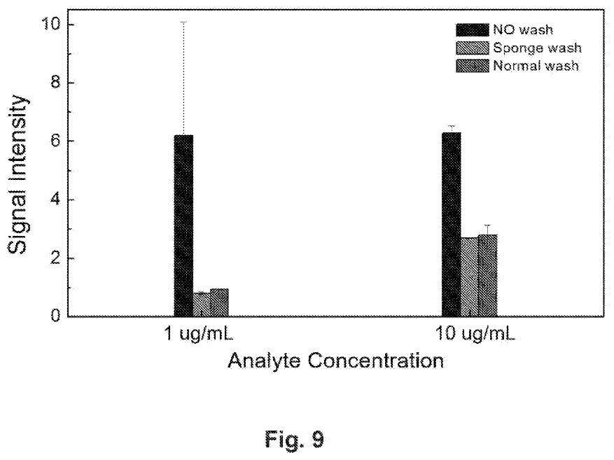

FIG. 9 and table 1 are summaries of an experimental realization of an exemplary embodiment of the present disclosure and indicate, according to the embodiment, relative performance of a squeeze-wash QMAX assay (samples 3 and 4 in Table 1) versus a QMAX assay with no washing (samples 1 and 2 in Table 1) and a QMAX assay with a conventional wash (samples 5 and 6 in Table 1).

TABLE 1

| # | cAb | dAb | Antigen | Wash method |
|---|---|---|---|---|
| 1 | Dry Mouse Anti- | Dry Goat Anti-IgG- | 10 uL 1 ug/mL | No |
| | IgG 20 ug/mL | IR800 20 ug/mL | Human IgG | |
| 2 | Dry Mouse Anti- | Dry Goat Anti-IgG- | 10 uL 10 ug/mL | No |
| | IgG 20 ug/mL | IR800 20 ug/mL | Human IgG | |
| 3 | Dry Mouse Anti- | Dry Goat Anti-IgG- | 10 uL 1 ug/mL | Sponge 1× |
| | IgG 20 ug/mL | IR800 20 ug/mL | Human IgG | 200 uL~300 |
| | | | | uL once |
| 4 | Dry Mouse Anti- | Dry Goat Anti-IgG- | 10 uL 10 ug/mL | Sponge 1× |
| | IgG 20 ug/mL | IR800 20 ug/mL | Human IgG | 200 uL~300 |
| | | | | uL once |
| 5 | Dry Mouse Anti- | Dry Goat Anti-IgG- | 10 uL 1 ug/mL | Regular 3× |
| | IgG 20 ug/mL | IR800 20 ug/mL | Human IgG | 150 uL |
| | | | | 3 min 3× |
| 6 | Dry Mouse Anti- | Dry Goat Anti-IgG- | 10 uL 10 ug/mL | Regular 3× |
| | IgG 20 ug/mL | IR800 20 ug/mL | Human IgG | 150 uL |
| | | | | 3 min 3× |

In the experiment, to prepare the sample, one plate was coated with: (1) protein-A for 2 hours, (2) CAb for 2 hours, and (3) blocking agent and stabilizer for 2 hours, the other plate was coated with dAb-L and stabilizer for 2 hours; the sample included an antigen of human IgG at 1 ug/ml, the incubation at the closed configuration was 5 min before the assay plate was washed. As shown in FIG. 9, sponge wash achieves the same signal as the conventional wash. It should be noted, however, that the sponge wash is much faster and much easier/simpler to conduct compared to the conventional wash. In the experiments shown in FIG. 9, the sponge wash took less than 30 seconds, the conventional wash took about 10 minutes. The samples that were not washed showed high signal but large variation (too high background signal), making the results unreliable.

FIG. 10 illustrates a squeeze-wash SCOF assay kit 12. The kit 12 includes an assay plate 22, one or more second plates 24, and a wash pad 40. The assay plate 22, the second plate(s) 24, and the wash pad 40 are sealed and/or environmentally stabilized (e.g., reagents are dried on the respective plates and/or contained in an environmental stabilization layer). As shown in FIG. 11, the wash pad 40 is sealed with a wash pad seal 146. The wash pad seal 146 is configured (in conjunction with the optional backing 140) to retain wash solution 44 within the wash pad 40, which is useful for example when distributing the wash pad in a kit 12.

In some embodiments, a device for washing a surface of a plate, comprising:

a first plate and a second plate, wherein:
i. the first plate is a plate that has a sample surface to be washed,
ii. the second plate is a plate that is made a porous material that has at least partial of the pores that are deformable and are capable of absorbing a solution by capillary force,
iii. the plates are movable relative to each other into different configurations,
iv. one or both plates are flexible,
v. one or both of the plates comprise spacers that are fixed with a respective plate, wherein the spacers have a predetermined substantially uniform height and a predetermined constant inter-spacer distance that is up to 250 um;

wherein one of the configurations is an open configuration, in which: the two plates are separated apart, the spacing between the plates is not regulated by the spacers, and the sample is deposited on one or both of the plates, and

wherein another of the configurations is a closed configuration which is configured after the sample deposition in the open configuration; and in the closed configuration: at least part of spacing between the two plates is regulated by the plates and the spacers.

During the operation, a wash solution was first filled into the pores of the porous material, and then bring the two plate into the closed configuration and deform the porous material to release the solution. The solution will be in the spacing between the plates, and will be absorbed back the porous material when the pressing force is released, and the pores turned to its original shape (the same or similar shape before the pressing).

The spaces can reduce the contact between the two surfaces of the plates at the closed configuration, and thereby reduce damages to the sample surface to be washed.

In some embodiments, the inter-spacer distance is in the range of 1 μm to 400 μm (e.g. 1 μm to 10 μm, 10 μm to 50 μm, 50 μm to 100 μm, 100 to 200 μm, 200 to 300 μm, or 300 to 400 μm).

In some embodiments, the spacer has a height in the range of 1 μm to 250 μm (e.g. 1 μm to 10 μm, 10 μm to 50 μm, 50 μm to 100 μm, 100 to 200 μm, or 200 to 250 μm); and a lateral dimension from 1 μm to 300 μm (e.g. 1 μm to 10 μm, 10 μm to 50 μm, 50 μm to 100 μm, 100 to 200 μm, or 200 to 300 μm), wherein a spacer will select one of the values respectively.

In some embodiments, the spacing are fixed on a plate by directly embossing the plate or injection molding of the plate.

In some embodiments, the materials of the plate and the spacers are selected from polystyrene, PMMA, PC, COC, COP, or another plastic.

In some embodiments, the spacers have a density of at least 100/mm², at least 1000/mm², or at least 10000/mm².

In some embodiments, the mold used to make the spacers is fabricated by a mold containing features that are fabricated by either (a) directly reactive ion etching or ion beam etched or (b) by a duplication or multiple duplication of the features that are reactive ion etched or ion beam etched.

### 2 Summary of Embodiments of Squeeze/Sponge-Wash Assay Methods, Kits, and Systems

The present invention includes a variety of embodiments, which can be combined in multiple ways as long as the various components do not contradict one another. The embodiments should be regarded as a single invention file: each filing has other filing as the references and is also referenced in its entirety and for all purpose, rather than as a discrete independent. These embodiments include not only the disclosures in the current file, but also the documents that are herein referenced, incorporated, or to which priority is claimed.

Examples of inventive subject matter according to the present disclosure are described in the following enumerated paragraphs.

### 2.1 A Squeeze/Sponge-Wash Assay Method

Embodiment 1: An assay method comprising, in order:

(a) placing a biological sample between an assay surface of an assay plate and an assay surface of a second plate, wherein the biological sample includes analyte molecules, wherein the assay surface of the assay plate includes an assay site that includes capture agents bound to the assay site, wherein the capture agents are configured to specifically associate the analyte molecules, and wherein at least one of the assay surface of the assay plate, the assay surface of the second plate, and the biological sample includes spacers sized to separate the assay plate and the second plate by a threshold thickness,

(b) squeezing the assay plate and the second plate together to a squeezed thickness regulated by the spacers,

(c) separating the assay plate and the Second plate,

(d) contacting the assay plate with a wash pad, having a wash surface, loaded with wash solution, wherein the wash surface is the surface of the wash pad that contacts the assay plate, and

(e) squeezing the Wash pad and the assay plate together to expel wash solution from the wash pad onto the assay site of the assay plate.

In the method of embodiment 1, the (a) placing includes placing the biological sample on at least one of the assay surface of the assay plate and the assay surface of the second plate.

In the method of embodiment 1, the (a) placing includes placing the biological sample on at least one of the assay surface of the assay plate and the assay surface of the second plate, and closing the biological sample between the assay plate and the second plate.

In the method of any of prior embodiments, the method further comprises, after the (a) placing and before the (c) separating, incubating the biological sample in contact with the capture agents for a period of time related to the saturation binding time of the analyte molecules to the capture agents.

In the method of any of prior embodiments, the period of time is less than 500 seconds, less than 100 seconds, less than 50 seconds, less than 20 seconds, less than 5 seconds, or less than 2 seconds.

In the method of any of prior embodiments, the assay plate includes a plurality of assay sites spaced apart a minimum site spacing, and the period of time is less than an average lateral diffusion time of the analyte molecules to traverse the minimum site spacing.

In the method of any of prior embodiments, the (b) squeezing includes squeezing the assay plate and the second plate together to accelerate a diffusion-limited reaction time of the analyte molecules to the capture agents relative to an un-squeezed sample.

In the method of any of prior embodiments, the assay surface of the second plate includes detection agents adhered to the assay surface and the detection agents are configured to Specifically associate at least one of the analyte molecule and the analyte molecule bound to the capture agent.

In the method of any of prior embodiments, the (b) squeezing includes squeezing the assay plate and the second plate together to accelerate a diffusion-limited reaction time of the detection agents to the analyte molecules relative to an un-squeezed sample.

In the method of any of prior embodiments, the (b) squeezing includes squeezing the assay plate and the second plate together to accelerate a diffusion-limited reaction time of the detection agents to the analyte molecules bound to the capture agents relative to an un-squeezed sample.

In the method of any of prior embodiments, the (d) contacting includes contacting at least part of the spacers with the wash pad loaded with the wash solution, wherein said part of the spacers and the wash surface are configured to prevent the direct contact between the wash surface and the assay site.

In the method of any of prior embodiments, before the (d) contacting, said part of the spacers are within and/or adjacent to the assay site.

In the method of any of prior embodiments, the wash surface is rigid.

In the method of any of prior embodiments, the wash pad includes wash pad spacers on the wash surface, the wash surface and the wash pad spacers are configured to prevent the direct contact between the wash surface and the assay site.

In the method of any of prior embodiments, after the (d) contacting, the wash pad spacers are within and/or adjacent to the assay site.

In the method of any of prior embodiments, the wash surface is rigid.

In the method of any of prior embodiments, the (d) contacting includes placing the Wash pad between the assay surface of the assay plate and the assay surface of the second plate.

In the method of any of prior embodiments, the (e) squeezing includes squeezing the Wash pad between the Second plate and the assay plate.

In the method of any of prior embodiments, the method further comprises removing wash pad from the assay plate after the (e) squeezing.

In the method of any of prior embodiments, the method further comprises covering the assay surface of the assay plate after removing the Wash pad, optionally by covering the assay plate with at least one of the second plate and a cover plate.

In the method of any of prior embodiments, the method further comprises, after the (e) squeezing, detecting analyte molecules bound to the capture agents.

In the method of any of prior embodiments, the detecting includes measuring at least one of fluorescence, luminescence, scattering, reflection, absorbance, and surface plasmon resonance associated with the analyte molecules bound to the capture agents.

In the method of any of prior embodiments, assay surface of the assay plate at the assay site includes a signal amplification surface such as a metal and/or dielectric microstructure (e.g., a disk-coupled dots-on-pillar antenna array).

In the method of any of prior embodiments, the (d) contacting includes contacting the assay site with the wash pad without wash solution and adding wash solution to the wash pad while in contact with the assay site to load the wash pad with wash solution.

In the method of any of prior embodiments, the method further comprises, before the (d) contacting, adding wash solution to the wash pad to load the wash pad with wash solution.

In the method of any of prior embodiments, the wash pad includes porous media configured to hold the wash solution.

In the method of any of prior embodiments, the porous media is configured to hold the wash solution in an open volume of the porous media.

In the method of any of prior embodiments, an/the open volume of the porous media is reduced upon compression of the porous media.

In the method of any of prior embodiments, the porous media is resiliently compressible, being configured to return to an uncompressed shape and an uncompressed open volume after an application and subsequent release of compression.

In the method of any of prior embodiments, the (e) squeezing includes diluting the sample and unbound analyte molecules with expelled wash solution.

In the method of any of prior embodiments, the (e) squeezing includes draining expelled wash solution from the wash pad and the assay plate.

In the method of any of prior embodiments, the method further comprises ceasing the (e) squeezing to permit the wash pad to absorb excess fluid into a/the porous media of the wash pad.

In the method of any of prior embodiments, the threshold thickness is at least 0.1 μm, at least 0.5 μm, or at least 1 μm.

In the method of any of prior embodiments, the squeezed thickness is at most 1 mm or at most 200 μm.

In the method of any of prior embodiments, the squeezed thickness is at most 20 μm, at most 10 μm, or at most 2 μm.

In the method of any of prior embodiments, the assay plate includes spacers.

In the method of any of prior embodiments, the second plate includes spacers.

In the method of any of prior embodiments, the biological sample does not include spacers.

A multi-step assay comprising:

performing the method of any of prior embodiments, the second plate is a first reagent plate that includes a first reagent on the assay surface and the wash pad is a first wash pad;
removing the first wash pad from the assay plate, and
performing the method of any of prior embodiments, the second plate is a second reagent plate that includes a second reagent on the assay surface and the wash pad is a second wash pad.

### 2.2 A Kit of Squeeze/Sponge-Wash Assay

Embodiment 2: A kit for assaying a sample, comprising:

a first plate, a second plate, and a sponge, wherein:
i. the plates are movable relative to each other into different configurations,
ii. the first plate comprises, on its inner surface, a sample contact area for contacting a sample that comprises an analyte,
iii. the sponge is made of a flexible porous material that has flexible pores with their shapes changeable under a force and that can absorb a liquid into the sponge or release a liquid out of the sponge, when the shape of the pores is changed;
   wherein one of the configurations is an open configuration, in which: the two plates are partially or completely separated apart, allowing the sample to be deposited on one or both of the plates,
   wherein another of the configurations is a closed configuration which is configured after the sample is deposited in the open configuration; and in the closed configuration: at least part of the sample is compressed by the two plates into a layer and is substantially stagnant relative to the plates, wherein the layer is confined by the inner surfaces of the two plates, and
   wherein the sponge is configured to deposit a wash solution that fills the sponge on the sample contact area when the sponge is pressed and re-absorb the wash solution when the pressing force is relieved.

Embodiment 3: A kit for assaying a sample, comprising:

a first plate, a second plate, spacers and a sponge, wherein:
i. the plates are movable relative to each other into different configurations,
ii. the first plate comprises, on its inner surface, a sample contact area for contacting a sample that comprises an analyte,
iii. the spacers are fixed on respective surfaces of one or both of the plates, wherein the spacers have a predetermined substantially uniform height and a predetermined fixed inter-spacer distance, and iv. the sponge is made of a flexible porous material that has flexible pores with their shapes changeable under a force and that can absorb a liquid into the sponge or release a liquid out of the sponge, when the shape of the pores is changed;
   wherein one of the configurations is an open configuration, in which: the two plates are partially or completely separated apart, the spacing between the plates is not regulated by the spacers, allowing the sample to be deposited on one or both of the plates,
   wherein another of the configurations is a closed configuration which is configured after the sample is deposited in the open configuration; and in the closed configuration: at least part of the sample is compressed by the two plates into a layer of highly uniform thickness and is substantially stagnant relative to the plates, wherein the uniform thickness of the layer is confined by the inner surfaces of the two plates and is regulated by the plates and the spacers, and
   wherein the sponge is configured to deposit a wash solution that fills the sponge on the sample contact area when the sponge is pressed and re-absorb the wash solution when the pressing force is relieved.

In the kit of embodiment 2 or 3, the kit further comprises a sponge container, which is configured to accommodate the sponge.

In the kit of any prior embodiment, the sponge comprises an enclosing wall with a sealed bottom that holds a solution in inside the sponge container.

In the kit of any prior embodiment, the pressing uses a plate and the bottom of the sponge container.

In the kit of any prior embodiment, the kit comprises multiple sponges.

In the kit of any prior embodiment, the kit comprises multiple containers.

In the kit of any prior embodiment, the kit comprises multiple sponges, which are configured to be accommodated by one container.

In the kit of any prior embodiment, the kit comprises a separate dry sponge for absorbing liquid only.

In the kit of any prior embodiment, the kit comprises a separate sponge for release liquid only.

In the kit of any prior embodiment, the sponge container further comprises a lid.

### 2.3 A Method for Squeeze/Sponge-Wash Assay

Embodiment 4: A method of sample analysis, comprising:

(a) obtaining a QMAX device that comprises a first plate and a second plate, which are movable into different configurations, including an open configuration and a closed configuration,
(b) depositing a liquid sample on a sample contact area of the first plate in the open configuration, in which the two plates are partly or entirely separated apart
(c) pressing the plates into a closed configuration, in which at least part of the sample is compressed into a layer of uniform thickness and incubating the sample for a predetermined period of time,
(d) removing the second plate,
(e) placing a sponge that contains a wash solution on the sample contact area of the first plate,
(f) pressing the sponge to deposit the wash solution onto the sample contact area, holding the sponge at the pressed position for a period of time, and releasing the sponge to reabsorb the wash solution.

In the method of Embodiment 4, the first plate or the second plate comprises spacers that are fixed on the respective surface.

In the method of Embodiment 4, the first plate or the second plate comprises spacers that are fixed on the respective surface and the spacers are configured to regulate the thickness of the sample between the first plate the second plate when the sample is compressed.

In the method of any prior embodiment, incubation period of time is less than 500 seconds, less than 100 seconds, less than 50 seconds, less than 20 seconds, less than 5 seconds, or less than 2 seconds, or in a range between any of the two values.

In the method of any prior embodiment, the inner surface of the second plate includes detection agents adhered to the assay surface and the detection agents are configured to specifically associate at least one of the analyte molecule and the analyte molecule bound to the capture agent.

In the method of any prior embodiment, the pressing in step (f) includes squeezing the sponge between the second plate and the first plate

In the method of any prior embodiment, the method further comprises removing the sponge from the first plate after the step (f).

In the method of any prior embodiment, the method further comprises repeating step (f) for one or more times.

In the method of any prior embodiment, the method further comprises reloading the sponge with fresh wash solution and repeat steps (e) and (f) for one or more times.

In the method of any prior embodiment, the sponge is made of a flexible porous material that has flexible pores with their shapes changeable under a force and that can absorb a liquid into the sponge or release a liquid out of the sponge, when the shape of the pores are changed.

### 2.4 A Device for Squeeze/Sponge-Wash Assay

Embodiment 5: A device for Sample analysis, comprising:

a first plate, a second plate, a third plate, and spacers, wherein:
i. the second plate and the third plate are respectively connected to the first plate, the second plate and the third plate are configured to each pivot against the first plate without interfering with each other,
ii. by pivoting against the first plate, either the second plate or the third plate is movable relative to the first plate into different configurations,
iii. the first plate comprises an inner surface that has a sample contact area for contacting a liquid Sample that Contains a component, and
iv. the spacers are fixed on one or more of the plates or are mixed in the sample, and
   wherein one of the configurations is an open configuration, in which: all three plates are partially or entirely separated apart and the spacing between the plates is not regulated by the spacers, and the sample is deposited on the first plate, the second plate, or both; and
   wherein another of the configurations is a closed configuration which is configured after the sample deposition in the open configuration, and in the closed configuration: at least part of the sample deposited is compressed by the first plate and the second plate into a layer of highly uniform thickness, which is confined by the inner surfaces of the first and second plates and is regulated by the plates and the spacers.

In the device of Embodiment 5, the device further comprises a sponge made of a flexible porous material.

In the device of any prior embodiment, the flexible porous material has pores with their shapes changeable under a force and that can absorb a liquid into the sponge or release a liquid out of the sponge, when the shape of the pores is changed.

In the device of any prior embodiment, the third plate is configured to press the sponge when the third plate pivots toward the first plate.

In the device of any prior embodiment, one edge of the second plate is connected to the inner surface of the first plate with a first hinge.

In the device of any prior embodiment, one edge of the third plate is connected to the inner surface of the first plate with a second hinge.

In the device of any prior embodiment, one edge of the second plate is connected to the inner surface of the first plate with a first hinge, and one edge of the third plate is connected to the inner surface of the first plate with a second hinge.

In the device of any prior embodiment, in the closed configuration between the first plate and second plate, the third plate can be adjusted to pivot against the first plate and the second plate.

In the device of any prior embodiment, the first plate comprises one or more notches on one or more of its edges, the notches are positioned such that the second plate and/or the third plate are juxtaposed on the notches to facilitate the manipulation of pivoting of the second plate and the third plate.

In the device of any prior embodiment, the second plate comprises a plate tab, which is configured to facilitate switching the plates between different configurations.

In the device of any prior embodiment, the sponge comprises a sponge tab, which is configured to facilitate removing the sponge from the plates.

### 2.5 A Kit for Sample Washing and Analysis

Embodiment 6: A kit for sample washing and analysis, comprising:

the device of Embodiment 5, and
a sponge that is made of a flexible porous material that has flexible pores with their shapes changeable under a force and that can absorb a liquid into the sponge or release a liquid out of the sponge, when the shape of the pores is changed.

In the kit of Embodiment 6, the sponge is configured to be pressed by the third plate when the sponge is positioned on the first plate.

In the kit of Embodiment 6 or any derived embodiment, wherein:
i. the sample comprises an analyte,
ii. a capture agent is Coated on a sample contact area in the first plate, and
iii. the capture agent is configured to specifically bind to the analyte.

In the kit of Embodiment 6 or any derived embodiment, one edge of the second plate is connected to the inner surface of the first plate with a first hinge, and one edge of the third plate is connected to the inner surface of the first plate with a second hinge.

In the kit of Embodiment 6 or any derived embodiment, in the closed configuration between the first plate and second plate, the third plate can be adjusted to pivot against the first plate and the second plate.

In the kit of Embodiment 6 or any derived embodiment, the kit further comprises a container, which is configured to accommodate the sponge.

In the kit of Embodiment 6 or any derived embodiment, the container contains washing medium.

In the kit of Embodiment 6 or any derived embodiment, the sponge comprises an enclosing wall with a sealed bottom that holds a solution in inside the sponge container.

### 2.6 A Method for Sample Analysis

Embodiment 7: A method of sample analysis, comprising:

(a) obtaining a device of Embodiment 5,
(b) depositing a liquid sample on inner surface of the first plate in the open configuration,
(c) pressing the second plates into the closed configuration,
(d) opening the second plate,
(e) placing a sponge that contains a wash solution on the inner surface of the first plate,
(f) pressing the sponge with the third plate to deposit the wash solution onto the inner surface of the first plate, holding the sponge at the pressed position for a period of time, and releasing the sponge to reabsorb the wash solution.

In the method of Embodiment 7, one edge of the second plate is connected to the inner surface of the first plate with a first hinge, and one edge of the third plate is connected to the inner surface of the first plate with a second hinge.

In the method of Embodiment 7 or any derived embodiment, the first plate comprises at least one assay site, the sample deposited on the assay site and the spacers are fixed to the assay site.

In the method of Embodiment 7 or any derived embodiment, the first plate comprises a capture reagent coated on the inner surface of the first plate, the capture reagent is configured to bind specifically to an analyte in the sample.

In the method of Embodiment 7 or any derived embodiment, the first plate comprises a plurality of assay sites spaced apart a minimum site spacing.

In the method of Embodiment 7 or any derived embodiment, further comprising: after the step (f), detecting the analyte bound to the capture agents.

In the method of Embodiment 7 or any derived embodiment, the detecting includes measuring at least one of fluorescence, luminescence, scattering, reflection, absorbance, and surface plasmon resonance associated with the analyte bound to the capture agents.

In the method of Embodiment 7 or any derived embodiment, the inner surface of the first plate at the assay site includes a signal amplification surface Such as a metal and/or dielectric microstructure (e.g., a disk-Coupled dots-On-pillar antenna array).

### 2.7 A Method for Performing an Assay

Embodiment 8: A method for performing an assay, comprising:

(a) obtaining a first plate comprising, on its inner surface, a sample contact area that has a first reagent site, wherein the first reagent site comprises a first reagent that bio/chemically interacts with a target analyte in a sample,
(b) obtaining a second plate comprising, on its inner surface, a sample contact area that has a second reagent site, wherein the second reagent site comprises a second reagent, that is capable of, upon contacting the sample, diffusing in the sample,
(c) obtaining a third plate comprising, on its inner surface, a sample contact area that has a third reagent site, wherein the third reagent site comprises a third regent, that is capable of, upon contacting a transfer liquid, diffusing in the transfer liquid,
(d) depositing, in an open configuration, the sample on one or both of the sample contact areas of the first and second plates,
(e) after (d), bringing the first and second plates to a closed configuration;
(f) after (e) separating the first and second plate,
(g) after (f) depositing, in an open configuration, a transfer liquid on one or both of the sample contact areas of the second and third plates,
(h) after g), bringing the second and third plates to a closed configuration; and
(i) detecting a signal related to the target analyte,
wherein the first, second, and third plates are movable relative to each other into different configurations, including an open and a closed configuration,
wherein in the open configuration, the sample contact areas of the two plates are separated larger than 200 um; and
wherein, in the closed configuration, at least part of the sample deposited in (d) or the transfer liquid deposited in (g) is confined between the sample contact areas of the two plates, and has an average thickness in the range of 0.01 to 200 μm.

### 2.8 A Kit, Device, and Method for Sample Analysis

Embodiment 9: The kit, device, and method of any prior embodiments, wherein the sponge comprises a porous substrate and said porous substrate contains pores of a diameter in the range of 10 nm to 100 nm, 100 nm to 500 nm, 500 nm to 1 μm to 10 μm, 10 μm to 50 μm, 50 μm to 100 μm, 100 μm to 500 μm, 500 μm to 1 mm.

In the kit, device, and method of Embodiment 9, the sponge comprises a porous substrate and said porous substrate contains pores of a diameter in the range of 500 nm to 1 μm, 1 μm to 10 μm, 10 μm to 50 μm, 50 μm to 100 μm, 100 μm to 500 μm.

In the kit, device, and method of any prior embodiments, the sponge comprises a porous Substrate and said porous Substrate possesses a porosity in the range of 10 to 20%, 20 to 30%, 30 to 40%, 40 to 50%, 50 to 60%, 60 to 70%, 70 to 80%, 80 to 90%, 90 to 99%.

In the kit, device, and method of any prior embodiments, said the sponge comprises a porous Substrate and said porous Substrate possesses a porosity in the range of 70 to 80%, 80 to 90%, 90 to 99%.

In the kit, device, and method of any prior embodiments, the sponge comprises a porous substrate and the materials of said porous substrate contains rubber, cellulose, cellulose wood fibers, foamed plastic polymers, low-density polyether, Polyvinyl alcohol (PVA), polyester, Poly(methyl methacrylate) (PMMA), polystyrene, etc.

In the kit, device, and method of any prior embodiments, the sponge comprises a porous substrate and said porous substrate is hydrophilic means the contact angle of sample droplet (e.g. water) on substrate is between 0 to 15 degree, 15 to 30 degree, 30 to 45 degree, 45 to 60 degree, 60 to 90 degree, with preferred contact angle of 15 to 30 degree, 30 to 45 degree, 45 to 60 degree.

In the kit, device, and method of any prior embodiments, said porous substrate is hydrophobic, the contact angle of sample droplet (e.g. water) on substrate is between 90 to 105 degree, 105 to 120 degree, 120 to 135 degree, 135 to 150 degree, 150 to 180 degree, with preferred contact angle of 105 to 120 degree, 120 to 135 degree, 135 to 150 degree.

In the kit, device, and method of any prior embodiments, said porous substrate is hydrophilic means the contact angle of sample droplet (e.g. water) on substrate is between 0 to 15 degree, 15 to 30 degree, 30 to 45 degree, 45 to 60 degree, 60 to 90 degree.

In the kit, device, and method of any prior embodiments, wherein:

iv. a capture agent is coated on the sample contact area, and
v. the capture agent is configured to specifically bind to the analyte.

In the kit, device, and method of any prior embodiments, the wash solution is deposited on the sample contact area after the binding of the analyte and the capture agent has reached an equilibrium.

In the kit, device, and method of any prior embodiments, the capture agent is an antibody, a DNA molecule or an RNA molecule.

In the kit, device, and method of any prior embodiments, either of the plates comprises at least one assay site on the respective sample contact area, the sample deposited on the assay site and the spacers are fixed to the assay site.

In the kit, device, and method of any prior embodiments, the second plate comprises a plate tab, which is configured to facilitate switch the plates between different configurations.

In the kit, device, and method of any prior embodiments, the sponge comprises a sponge tab, which is configured to facilitate removing the sponge from the plates.

In the kit, device, and method of any prior embodiments, the sponge is configured to:
(i) contain, before being pressed, a washing solution inside the sponge,
(ii) release, when being pressed, at least a part of the washing solution, and
(iii) absorb, when the pressing is completed, at least a part of the liquid released.

In the kit, device, and method of any prior embodiments, the spacers are fixed on the first plate.

In the kit, device, and method of any prior embodiments, the spacers are fixed on both the first and second plates.

In the kit, device, and method of any prior embodiments, the sample is whole blood and the component are blood cells.

In the kit, device, and method of any prior embodiments, the first plate comprises a reagent site on its sample contact area.

In the kit, device, and method of any prior embodiments, the second plate comprises a reagent site on its sample contact area.

In the kit, device, and method of any prior embodiments, the sponge contains a washing solution.

In the kit, device, and method of any prior embodiments, the sponge contains a solution.

In the kit, device, and method of any prior embodiments, the sponge contains a liquid reagent.

### 3 Assay Dilution Calibration

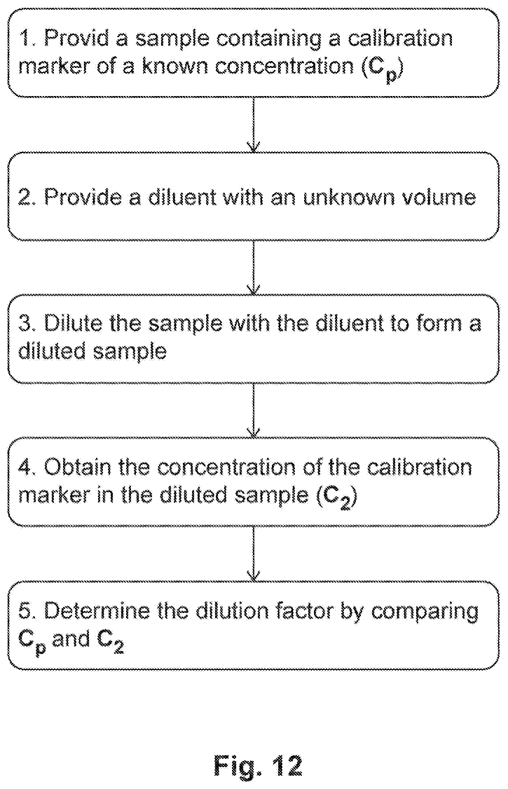

FIG. 12 is a flow diagram of an exemplary embodiment of a method of determining the dilution factor for a sample provided by the present invention. The method comprises:
(i) providing a sample containing a calibration marker, the calibration marker having a concentration that is known as a preset value Cp,
(ii) providing a diluent with an unknown volume,
(iii) diluting the sample with the diluent to form a diluted sample;
(iv) obtaining, after (iii), a second value C₂ using a concentration-measuring tool, the second value being the concentration of the calibration marker in the diluted sample; and
(v) determining the dilution factor for the diluted sample by Comparing the preset value Cp and the Second value C₂.

In some embodiments, the preset value Cp may be a predetermined value that is the real concentration of the calibration marker in the sample. In other embodiments, the preset value Cp may be an assumed normal value based on past experiences, standards in the art, or other reasons, and Such a normal value is not too much different from the real concentration of the calibration marker in the sample. In some embodiments, such a difference between the preset value and the real concentration is 20% or less, 15% or less, 10% or less, 5% or less, 2.5% or less.

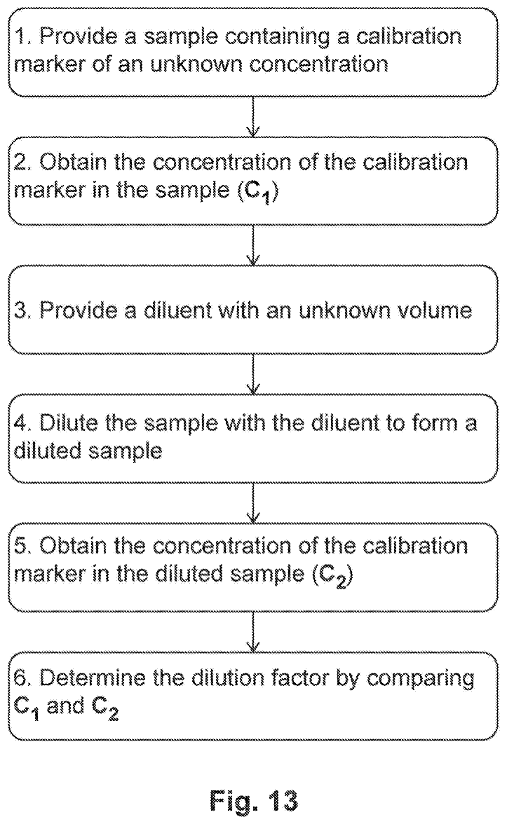

FIG. 13 is a flow diagram of another exemplary embodiment of a method of determining the dilution factor for a sample provided by the present invention. The method comprises:
(i) providing a sample containing a calibration marker, the calibration marker having an unknown concentration;
(ii) providing a diluent with an unknown volume,
(iii) obtaining a first value C₁ using a concentration-measuring tool, the first value being the concentration of the calibration marker in the sample,
(iv) diluting the sample with the diluent to form a diluted sample;
(v) obtaining, after (iv), a second value C₂ using the concentration-measuring tool, the second value being the concentration of the calibration marker in the diluted sample; and
(vi) determining the dilution factor by comparing the first value C₁ and the second value C₂.

As the order illustrated in FIG. 13, in some embodiments, the method may comprise: first obtaining the first value C₁ and then diluting the sample with the diluent to form the diluted sample.

It is to note, however, in other embodiments, the method may comprise a step before the steps of obtaining the first value C₁ and diluting the sample: dividing the sample into at least two portions:

a first portion and a second portion, the first portion to be used for the step of obtaining the first value C₁ and the second portion to be diluted with the diluent to form the diluted sample.

Although the present invention may be particularly useful when the volume of the diluent is unknown to the user of the method, in some embodiments, it is also applicable for situations when the volume of the diluent is known to the user of the method.

In some embodiments, the step of diluting the sample may be a single step of mixing the sample with the diluent, which may be a single foreign matter or a mixture of a plurality of foreign matters. In other embodiments, the diluting step may be a series of dilution steps, in which the sample is sequentially mixed with a plurality of foreign matters.

### 3.1 Definition

### Sample

The term “sample” as used herein generally refers to a material or mixture of materials containing one or more analytes of interest. In some embodiments of the present invention, the Sample may be one or any combination of a biological sample, an environmental sample, and a foodstuff sample.

In some embodiments, the sample may be obtained from a biological sample such as cells, tissues, bodily fluids, and stool. Typically, samples that are not in liquid form are converted to liquid form before analyzing the sample with the present method. Bodily fluids of interest include but are not limited to, amniotic fluid, aqueous humour, vitreous humour, blood (e.g., whole blood, fractionated blood, plasma, serum, etc.), breast milk, cerebrospinal fluid (CSF), cerumen (earwax), chyle, chime, endolymph, perilymph, feces, gastric acid, gastric juice, lymph, mucus (including nasal drainage and phlegm), pericardial fluid, peritoneal fluid, pleural fluid, pus, rheum, saliva, sebum (skin oil), semen, sputum, sweat, synovial fluid, tears, vomit, urine and exhaled condensate. In particular embodiments, a sample may be obtained from a Subject, e.g., a human, and it may be processed prior to use in the Subject assay. For example, prior to analysis, the protein/nucleic acid may be extracted from a tissue sample prior to use, methods for which are known. In particular embodiments, the sample may be a clinical sample, e.g., a sample collected from a patient.

In other embodiments, the sample may be obtained from an environmental sample, including, but not limited to: liquid samples from a river, lake, pond, Ocean, glaciers, icebergs, rain, Snow, Sewage, reservoirs, tap water, drinking water, etc.; solid samples from soil, compost, sand, rocks, concrete, wood, brick, sewage, etc.; and gaseous samples from the air, underwater heat vents, industrial exhaust, vehicular exhaust, etc. Typically, samples that are not in liquid form are converted to liquid form before analyzing the sample with the present method. in yet other embodiments, the sample may be obtained from a food sample that is suitable for animal consumption, e.g., human consumption. A foodstuff sample may if include, but not limited to, raw ingredients, cooked food, part and animal sources of food, preprocessed food as well as partially of fully processed food, etc. Typically, samples that are not in liquid form are converted to guide for in before analyzing the sample with the present method.

### Calibration Marker

The term “calibration marker” as used herein refers to any analyte contained in the sample, the detectable amount of which is not affected by the addition of the diluent. Here, the term “detectable amount” refers to the amount of the analyte that is detected by the calibration measuring tool provided in the method. Therefore, in some embodiments, under certain circumstances when the diluent is neutral to the sample (i.e. different matter from the sample and the components thereof and with no physical, chemical, or biological impact on the sample whatsoever), the calibration marker may be any analyte contained in the sample, such as, but not limited to, proteins, peptides, DNAS, RNAS, nucleic acids, inorganic molecules and ions, organic small molecules, cells, tissues, viruses, nanoparticles with different shapes, and any combination thereof.

In other embodiments, if the diluent is not neutral to the sample, the calibration marker may be chosen from the analytes contained in the sample based on the physical, chemical, and/or properties of both the analytes and the diluent.

More details of the analytes that may be used as calibration markers have been given in U.S. Provisional Application Ser. No. 62/202,989, filed on Aug. 10, 2015, 62/218,455 filed on Sep. 14, 2015, 62/293,188, filed on Feb. 9, 2016, and 62/305,123, filed on Mar. 8, 2016, the complete disclosures of which are hereby incorporated by references for all purposes.

### 3.2 Use of QMAX Device

The concentration-measuring tool in the method of the present invention may be any type of device or apparatus that determines the concentration of the calibration marker in the sample or diluted sample accordingly. In some embodiments, it may comprise a first part that determines the volume (V) of a part or entirety of the sample to be analyzed, a second part that determines the amount of the calibration marker (CM) contained with the part or entirety of the sample, and a third part configured to calculate the concentration of the calibration marker (ICM)) based on the determined value of V and CM, CM)=CM/V.

In some embodiments of the present invention, the concentration-measuring tool may be a CROF (compressed regulated open flow) device, or otherwise named QMAX (Q: quantitative, M. multiplexing, A. adding reagents, and X: acceleration) device, such as, but not limited to, the CROF device and QMAX device disclosed in U.S. Provisional Patent Application No. 62/202,989, which was filed on Aug. 10, 2015, U.S. Provisional Patent Application No. 62/218,455, which was filed on Sep. 14, 2015, U.S. Provisional Patent Application No. 62/293,188, which was filed on Feb. 9, 2016, U.S. Provisional Patent Application No. 62/305,123, which was filed on Mar. 8, 2016, U.S. Provisional Patent Application No. 62/369,181, which was filed on Jul. 31, 2016, U.S. Provisional Patent Application No. 62/394,753, which was filed on Sep. 15, 2016, PCT Application (designating U.S.) No. PCT/US2016/045437, which was filed on Aug. 10, 2016, PCT Application (designating U.S.) No. PCT/US2016/051775, which was filed on Sep. 14, 2016, PCT Application (designating U.S.) No. PCT/US2016/051794, which was filed on Sep. 15, 2016, and PCT Application (designating U.S.) No. PCT/US2016/054025, which was filed on Sep. 27, 2016, the complete disclosures of which are hereby incorporated by reference for all purposes.

In some embodiments, a QMAX device comprises:

a first plate and a Second plate, wherein:
i. the plates are movable relative to each other into different configurations;
ii. one or both plates are flexible;
iii. each of the plates has, on its respective surface, a sample contact area for contacting a sample with an analyte,
iv. one or both of the plates comprise spacers that are fixed with a respective plate, wherein the spacers have a predetermined substantially uniform height and a predetermined constant inter-spacer distance and wherein at least one of the spacers is inside the sample contact area; and
   a detector that detects the analyte;
   wherein one of the configurations is an open configuration, in which: the two plates are separated apart, the spacing between the plates is not regulated by the spacers, and the sample is deposited on one or both of the plates; and
   wherein another of the configurations is a closed configuration which is configured after the sample deposition in the open configuration; and in the closed configuration: at least part of the sample is compressed by the two plates into a layer of highly uniform thickness and is substantially stagnant relative to the plates, wherein the uniform thickness of the layer is confined by the inner surfaces of the two plates and is regulated by the plates and the spacers, and has an average thickness equal to or less than 5 um with a small variation; and
   wherein at the closed configuration, the detector detects the analyte in the at least part of the sample.

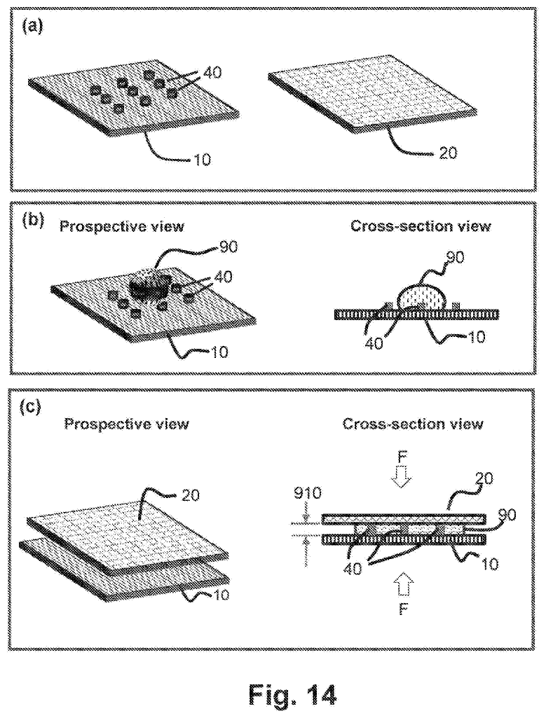

FIG. 14 shows an embodiment of a QMAX device, which comprises a first plate 10 and a second plate 20. In particular, panel (A) shows the perspective view of a first plate 10 and a second plate 20 wherein the first plate has spacers. It should be noted, however, that the spacers may also be fixed on the second plate 20 (not shown) or on both first plate 10 and second plate 20 (not shown). Panel (B) shows the perspective view and a sectional view of depositing a sample 90 on the first plate 1 at an open configuration. It should be noted, however, that the sample 90 may also be deposited on the second plate 20 (not shown), or on both the first plate 10 and the second plate 20 (not shown). Panel (C) illustrates (i) using the first plate 10 and second plate 20 to spread the sample 90 (the sample flow between the inner surfaces of the plates) and reduce the sample thickness, and (ii) using the spacers and the plate to regulate the sample thickness at the closed configuration of the QMAX device. The inner surfaces of each plate may have one or a plurality of binding sites and or storage sites (not shown).

In some embodiments, the spacers 40 have a predetermined uniform height and a predetermined uniform inter-spacer distance. In the closed configuration, as shown in panel (C) of FIG. 14, the spacing between the plates and the thus the thickness of the sample 910 is regulated by the spacers 40. In some embodiments, the uniform thickness of the sample 910 is substantially similar to the uniform height of the spacers 40.

In some embodiments of the present invention, when the QMAX device is used to obtain the first value, the obtaining step may comprise:

(a) obtaining the concentration-measuring tool, i.e. the QMAX device;
(b) depositing the sample on the sample contact area of one or both of the plates in the open configuration;
(c) compressing a relevant volume of the deposited sample into a layer of uniform thickness by bringing the two plates into the closed configuration;
(d) determining the amount of the calibration marker in a part or an entirety of the layer of thickness by detecting the calibration marker using the detector;
(e) estimating the volume of said part or entirety of the layer of thickness by timing the pre-determined uniform height of the spacers and the lateral area of said part or entirety of the layer of uniform thickness;
(f) obtaining the first value by dividing the determined amount of the calibration marker in step (d) by the estimated volume in step (e).

In some embodiments, when the QMAX device is used to obtain the second value, the obtaining step may comprise similar steps as above except that the diluted sample is the material to be deposited, compressed, and analyzed instead of the sample.

### 3.3 Determination of Dilution Factor for Blood Sample

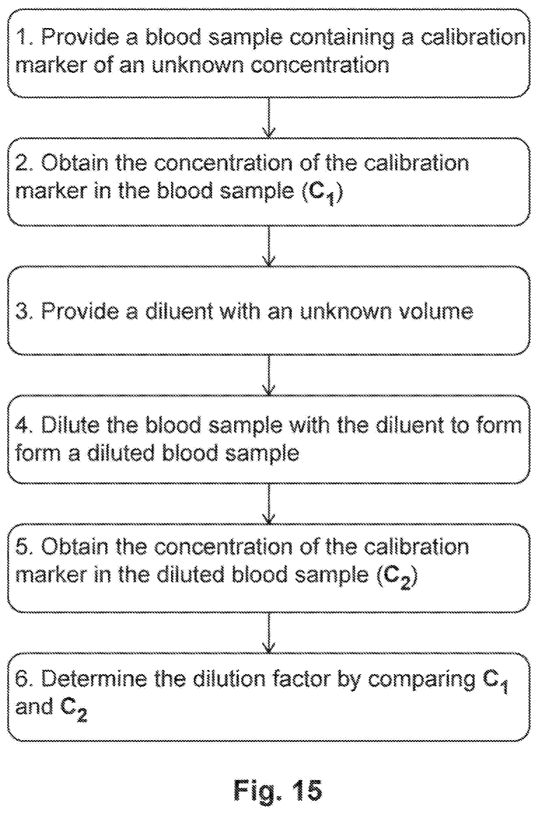

FIG. 15 is a flow diagram of an exemplary embodiment of a method to determine the dilution factor for a blood Sample, according to the present invention. The method comprises:
(i) providing a blood sample containing a calibration marker, the calibration marker having an unknown concentration;
(ii) obtaining a first value C₁ using a concentration measuring tool, the first value being the concentration of the calibration marker in the blood sample;
(iii) providing a diluent with an unknown volume,
(iv) diluting the sample with the diluent to form a diluted blood sample;
(v) obtaining, after (iv), a second value C₂ using a concentration measuring tool, the second value being the concentration of the calibration marker in the diluted blood Sample; and
(vi) determining the dilution factor by comparing the first value C₁ and the second value C₂.
   As disclosed above, when determining the dilution factor for a blood sample, the calibration marker may be selected from the any of the analytes contained in the blood sample, as long as the addition of the diluent has no physical, chemical, or biological impact on the detectable amount of the calibration marker. One or any combination of a group, comprising: red blood cells (RBCs), white blood cells (WBCS), and platelets (PLTs).

According to some embodiments of the present invention, a QMAX device may be used to measure the concentration of RBCs, WBCS, and/or PLTs before and after diluting the blood sample. The method of using QMAX device to determine the concentration of RBCs, WBCS, and/or PLTs includes, but not limited to, the ones disclosed in U.S. Provisional Patent Application No. 62/202,989, which was filed on Aug. 10, 2015, U.S. Provisional Patent Application No. 62/218,455, which was filed on Sep. 14, 2015, U.S. Provisional Patent Application No. 62/293,188, which was filed on Feb. 9, 2016, U.S. Provisional Patent Application No. 62/305,123, which was filed on Mar. 8, 2016, U.S. Provisional Patent Application No. 62/369,181, which was filed on Jul. 31, 2016, U.S. Provisional Patent Application No. 62/394,753, which was filed on Sep. 15, 2016, PCT Application (designating U.S.) No. PCT/US2016/045437, which was filed on Aug. 10, 2016, PCT Application (designating U.S.) No. PCT/US2016/051775, which was filed on Sep. 14, 2016, PCT Application (designating U.S.) No. PCT/US2016/051794, which was filed on Sep. 15, 2016, and PCT Application (designating U.S.) No. PCT/US2016/054.025, which was filed on Sep. 27, 2016, the complete disclosures of which are hereby incorporated by reference for all purposes.

### 3.4 Example: Determination of Dilution Factor for Human Blood Sample Using RBCs and WBCs

As disclosed in the experiments below, exemplary devices and methods for determining dilution factor for a human blood sample have been achieved. In these experiments, a fresh human blood sample was obtained and diluted in saline solution by different pre-determined dilution factors. RBCs and WBCs were used as calibration markers respectively to determine the dilution factor in each diluted blood sample. Briefly, their concentrations in all samples, including the undiluted and diluted blood samples, were measured using QMAX devices.

Dilution factor for each diluted sample was hence determined using the measured concentrations of RBCs and WBCS, respectively. Last, to examine the quality of the calculated dilution factors, they were compared against the pre-determined dilution factors for each diluted sample. The fact that the calculated dilution factors all showed close resemblance to the pre-determined dilution factors for each diluted sample clearly testifies to the validity of the methods and devices provided in the present invention.

### E-1. Materials and Methods

QMAX Device:

The QMAX device used in this experiment contained: 1) a planar glass substrate plate (25.4 mm×25.4 mm surface, 1 mm thick), and 2) an X-plate that is a planar PMMA plate (25.4 mm×25.4 mm surface, 175 um thick) having, on one of its surfaces, a periodical array of spacer pillars with 80 um spacing distance. Each spacer pillar is in rectangular shape with nearly uniform Cross-section and rounded Corners (lateral surface: 30 um×40 um, height: 2 um).

Acridine Orange Dye:

acridine orange (AO) is a stable dye that has natural affinity for nucleic acids. When binding to DNA, AO intercalates with DNA as a monomer and yields intense green fluorescence under blue excitation. (470 nm excitation, 525 nm green emission for white blood cells (WBCs)). When binding to RNAs and proteins it forms an electrostatic complex in a polymeric form that yields red fluorescence under blue excitation. (470 nm excitation, 685 nm red emission for WBCs and platelets (PLTs)). As a result, red blood cells (RBCs) were not stained because they have no nuclei and therefore little nucleic acids; WBCs were strongly stained because they have significant amount of nucleic acids; PLTs were weakly stained for the slight amount of RNAs they have.

Sample Processing, Dilution and Imaging:

Fresh human blood sample was obtained by pricking a finger of a human subject and then stained with AO dye. Briefly, it was mixed with AO (100 ug/mL in PBS) at 1:1 ratio for 1 min.

After staining, the sample was split into five parts, among which one part was labeled “Undiluted sample”, and each of the remaining parts was diluted with 0.9% sodium chloride solution at one of the following ratios: 1:2 (“2× diluted sample”), 1:5 (“5× diluted sample”), 1:10 (“10× diluted sample”), 1:20 (“2O× diluted sample”).

1 uL of each blood sample was transferred onto the center of the substrate plate using an Eppendorf pipette, and an X-plate was then placed on top of the substrate plate that bears the blood drop, with the spacer pillars facing toward the blood drop on the substrate plate, covering most area of the substrate plate. Next, the two plates were pressed against each other by a human hand uniformly for 10 sec and then released, after which the two plates were self-held in the same configuration, likely due to forces between the two plates, like capillary force.

An imaging system, composed of a commercial DSLR camera (Nikon), two filters, a light source and a magnification/focus lens set, was used to take pictures of the blood sample deposited in between the two plates in bright field mode and in fluorescence mode, to count RBCs and WBCs, respectively. In bright field mode, a broadband white light Xenon lamp source without any filter was used. In fluorescence mode, the excitation source was a Xenon lamp with a 470 it 20 nm excitation filter (Thorlabs), and the emission filter was a 500 nm long pass filter (Thorlabs).

### E-2. Results and Discussion

Here dilution factor for each diluted human blood sample was determined using the methods and QMAX devices provided by some embodiments of the present invention.

1. The concentrations of RBCs and WBCS in each sample, including the undiluted and the serially diluted samples, were measured using QMAX devices.

Number:

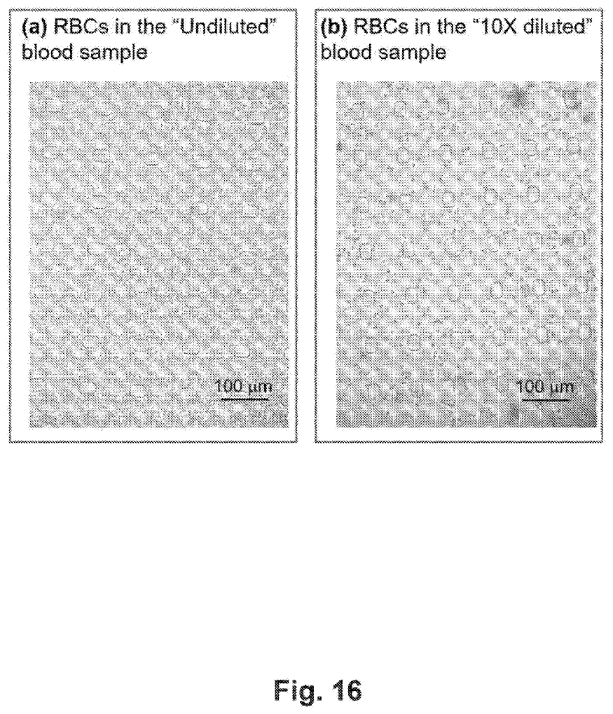

RBCs deposited in the QMAX devices were Counted in a relevant volume in bright field mode, while WBCS were Counted in fluorescence mode. FIG. 16 shows representative images of Undiluted (a) and 10× diluted (b) samples obtained in bright field mode. From the images, RBCs are readily recognizable, as defined by their Contrasted dark round boundary and relatively brighter Center, while the periodically aligned rounded rectangles are the spacer pillars on the X-plate. It is to be noted that the number of RBCs in FIG. 16(a) clearly appears less than FIG. 16(b), suggesting that 10× diluted sample was indeed more diluted than undiluted sample, with a lower concentration of RBCS.

Volume:

Given that the distance between the two plates was the height of the pillars when the two plates were hand-pressed to enter the device's closed configuration, the relevant volume of the deposited sample were readily calculated based on the pre-determined size, height, and pattern of the spacer pillar array.

Concentration:

The concentration of RBCs (RBCS) in each sample was then quantified as the quotient of the measured number of RBCs and the relevant volume, as summarized in Table A1, and the concentration of WBCs (WBCS) in each sample was quantified similarly using the Count of WBCs in the relevant volume (Table A2).

2. Dilution factor for each diluted sample was determined using the concentrations of RBCs and WBCS, respectively (Table A1 and A2). Specifically, to calculate dilution factor based on RBCs, the measured concentration of RBCs in each diluted sample was compared with their concentration in the undiluted sample (Table A1, N/A=not applicable). For dilution factor from WBCS, the measured concentration of WBCS in each diluted sample was compared with their concentration in the undiluted sample (Table A2, N/A=not applicable).

3. The dilution factors calculated from RBCs and WBCs were then compared against the pre-determined dilution factor in each sample, respectively. The percentage difference for method using RBCs (dilution factor calculated from RBCs—predetermined dilution factor/predetermined dilution factor*100%) was calculated for each diluted sample (Table A2). Percentage differences for method using WBCs (dilution factor calculated from WBCS—predetermined dilution factor/predetermined dilution factor*100%) were also calculated (Table A2). As shown in Table A1 and A2, none of the percentage differences exceeded 5%, demonstrating the validity of the methods and device for determining dilution factor provided in the present invention.

TABLE A1
concentrations of RBCS and calculated dilution factors

| | RBCs | Dilution factor | Percentage |
|---|---|---|---|
| | (/uL) | Calculated from RBCs | Difference (%) |
| undiluted | 4.90E+06 | N/A | N/A |
| 2X | 2.44E+06 | 1:2.01 | 0.41 |
| 5X | 9.70E+05 | 1:5.05 | 1.03 |
| 10X | 5.00E+05 | 1:9:80 | 2.00 |
| 20X | 2.50E+05 | 1:19.6 | 2.00 |

TABLE A2
concentrations of WBCS and calculated dilution factors

| | WBCs | Dilution factor | Percentage |
|---|---|---|---|
| | (/uL) | Calculated from WBCs | Difference (%) |
| undiluted | 8792 | N/A | N/A |
| 2X | 4432 | 1:1.98 | 0.81 |
| 5X | 1770 | 1:4.97 | 0.66 |
| 10X | 870 | 1:10.1 | 1.06 |
| 20X | 420 | 1:20.9 | 4.67 |

To summarize, the methods and device for determining dilution factor in human blood sample were examined in the above exemplary experiments, involving the use of RBCs and WBCs as calibration markers separately and the use of QMAX devices. The resultant dilution factors showed clear resemblance to the pre-determined dilution factor for each diluted sample, demonstrating the validity of the method and device provided in the present invention.

### 4 Summary of Embodiments for Assay Dilution Calibration

The present invention includes a variety of embodiments, which can be combined in multiple ways as long as the various components do not contradict one another. The embodiments should be regarded as a single invention file: each filing has other filing as the references and is also referenced in its entirety and for all purpose, rather than as a discrete independent. These embodiments include not only the disclosures in the current file, but also the documents that are herein referenced, incorporated, or to which priority is claimed.

### 4.1 A Method for Determining a Dilution Factor for a Diluted Sample

Embodiment 10: A method for determining a dilution factor for a diluted sample, comprising the steps of:
(i) providing an initial sample containing a calibration marker, the calibration marker having a first concentration with a known preset value;
(ii) diluting the initial sample with an unknown volume of a diluent to form a diluted sample;
(iii) obtaining, after (ii), a second concentration of the calibration marker in the diluted sample using a concentration-measuring device; and
(iv) determining the dilution factor for the diluted sample by comparing the first concentration and the second concentration.

In the method of Embodiment 10, the preset value is an estimated normal value, which is different from a true value of the first concentration by less than 5%.

Embodiment 11: A method for determining a dilution factor for a diluted sample, comprising the steps of:
(i) providing an initial sample containing a calibration marker, the calibration marker having an unknown concentration;
(ii) obtaining a first concentration of the calibration marker in the initial sample using a concentration-measuring device,
(iii) diluting the initial sample with an unknown volume of a diluent to form a diluted sample;
(iv) obtaining, after (iii), a second concentration of the calibration marker using the concentration-measuring device; and
(v) determining the dilution factor by Comparing the first concentration and the second concentration.

In the method of Embodiment 10 or Embodiment 11, the initial sample is made of a material selected from a group consisting of cells, tissues, stool, amniotic fluid, adueous humour, vitreous humour, blood (e.g., whole blood, fractionated blood, plasma, serum, etc.), breast milk, cerebrospinal fluid (CSF), cerumen (earwax), chyle, chime, endolymph, perilymph, feces, gastric acid, gastric juice, lymph, mucus (including nasal drainage and phlegm), pericardial fluid, peritoneal fluid, pleural fluid, pus, rheum, saliva, sebum (skin oil), semen, sputum, sweat, synovial fluid, tears, vomit, urine, and exhaled condensate.

In the method of any prior embodiment, the sample is an environmental liquid sample for a source selected from a group consisting of river, lake, pond, ocean, glaciers, icebergs, rain, show, sewage, reservoirs, tap water, or drinking water, solid samples from soil, compost, sand, rocks, concrete, wood, brick, sewage, and any combination thereof.

In the method of any prior embodiment, the sample is an environmental gaseous sample from a source selected from a group consisting of the air, underwater heat vents, industrial exhaust, vehicular exhaust, and any combination thereof.

In the method of any prior embodiment, the sample is a foodstuff sample selected from a group Consisting of raw ingredients, Cooked food, paint and a final Sources of food, preprocessed food, and partially or fully processed food, and any combination thereof.

In the method of any prior embodiment, the calibration marker is Selected from a group consisting of: proteins, peptides, DNAS, RNAS, nucleic acids, inorganic molecules and ions, organic small molecules, cells, tissues, viruses, nanoparticles with different shapes, and any combination thereof.

In the method of any prior embodiment, the concentration measuring device comprises: a first plate and a second plate, wherein:
v. the plates are movable relative to each other into different configurations;
vi. one or both plates are flexible;
vii. each of the plates has, on its respective surface, a sample contact area for contacting a sample that contains an analyte,
viii. One or both of the plates comprise spacers that are fixed with a respective plate, wherein the spacers have a predetermined substantially uniform height and a predetermined constant inter-spacer distance and wherein at least one of the spacers is inside the sample contact area; and
   a detector that detects the analyte;
   wherein one of the configurations is an open configuration, in which: the two plates are partially or entirely separated apart, the spacing between the plates is not regulated by the spacers, and the sample is deposited on one or both of the plates; and
   wherein another of the configurations is a closed configuration which is configured after the deposition of the sample in the open configuration; and in the closed configuration: at least part of the sample is compressed by the two plates into a layer of highly uniform thickness and is substantially stagnant relative to the plates, wherein the layer of uniform thickness is confined by the inner surfaces of the two plates and is regulated by the plates and the spacers, and has an average thickness equal to or less than 5 um with a small variation;
   wherein in the closed configuration, the detector detects the analyte in the at least part of the sample and calculates a concentration of the analyte in the sample.

In the method of any prior embodiment, the step of obtaining the first concentration comprises:

(a) obtaining the concentration-measuring device;
(b) depositing the initial sample on the sample contact area of one or both of the plates in the open configuration;
(c) compressing a relevant volume of the deposited initial sample into a layer of uniform thickness by bringing the two plates into the closed configuration;
(d) determining the amount of the calibration marker in a part or an entirety of the layer of thickness by detecting the calibration marker using the detector;
(e) estimating the volume of said part or entirety of the layer of thickness by timing the pre-determined uniform height of the spacers and the lateral area of said part or entirety of the layer of uniform thickness;
(f) obtaining the first concentration by dividing the determined amount of the calibration marker in step (d) by the estimated volume in step (e).

In the method of any prior embodiment, the step of obtaining the second concentration comprises:

(a) obtaining the concentration-measuring device;
(b) depositing the diluted sample on the sample contact area of one or both of the plates in the open configuration;
(c) compressing a relevant volume of the deposited diluted sample into a layer of uniform thickness by bringing the two plates into the closed configuration;
(d) determining the amount of the calibration marker in a part or an entirety of the layer of thickness by detecting the calibration marker using the detector;
(e) estimating the volume of said part or entirety of the layer of thickness by timing the pre-determined uniform height of the spacers and the lateral area of said part or entirety of the layer of uniform thickness;
(f) obtaining the second concentration by dividing the determined amount of the calibration marker in step (d) by the estimated volume in step (e).

### 4.2 A Method for Determining a Dilution Factor for a Blood Sample

Embodiment 12: A method for determining dilution factor for a blood sample, comprising:
(i) providing an initial blood sample containing a calibration marker, the calibration marker having an unknown concentration;
(ii) obtaining a first concentration of the calibration marker in the initial blood sample using a concentration measuring device,
(iii) diluting the initial blood sample with an unknown volume of a diluent to form a diluted blood
Sample;
(iv) obtaining, after (iv), a second concentration of the calibration marker in the diluted blood sample using a concentration measuring device; and
(v) determining the dilution factor by comparing the first concentration and the second concentration.

In the method of Embodiment 12, the calibration marker is selected from a group consisting of: red blood cells, white blood cells, platelets, and any combination thereof.

In the method of Embodiment 12 or any of its derived embodiments, the concentration-measuring device comprises:

a first plate and a second plate, wherein:
i. the plates are movable relative to each other into different configurations;
ii. one or both plates are flexible;
iii. each of the plates has, on its respective surface, a sample contact area for contacting a sample with an analyte,
iv. one or both of the plates comprise spacers that are fixed with a respective plate, wherein the spacers have a predetermined substantially uniform height and a predetermined constant inter-spacer distance that is in the range of 7 μm to 200 μm and wherein at least one of the spacers is inside the sample contact area, and has an average thickness equal to or less than 5 μm with a small variation; and
   a detector that detects the analyte;
   wherein one of the configurations is an open configuration, in which: the two plates are separated apart, the spacing between the plates is not regulated by the spacers, and the sample is deposited on one or both of the plates; and
   wherein another of the configurations is a closed configuration which is configured after the sample deposition in the open configuration; and in the closed configuration: at least part of the sample is compressed by the two plates into a layer of highly uniform thickness and is substantially stagnant relative to the plates, wherein the uniform thickness of the layer is confined by the inner surfaces of the two plates and is regulated by the plates and the spacers; and
   wherein at the closed configuration, the detector detects the analyte in the at least part of the sample.

In the method of Embodiment 12 or any of its derived embodiments, the step of obtaining the first concentration comprises:

(a) obtaining the concentration-measuring device;
(b) depositing the initial blood sample on the sample contact area of one or both of the plates in the open configuration;
(c) compressing a relevant volume of the deposited initial blood sample into a layer of uniform thickness by bringing the two plates into the closed configuration;
(d) determining the amount of the calibration marker in a part or an entirety of the layer of thickness by detecting the calibration marker using the detector;
(e) estimating the volume of said part or entirety of the layer of thickness by timing the pre-determined uniform height of the spacers and the lateral area of said part or entirety of the layer of uniform thickness;
(f) obtaining the first concentration by dividing the determined amount of the calibration marker in step (d) by the estimated volume in step (e).

In the method of Embodiment 12 or any of its derived embodiments, the step of obtaining the second concentration comprises:

(a) obtaining the concentration-measuring device;
(b) depositing the diluted blood sample on the sample contact area of one or both of the plates in the open configuration;
(c) compressing a relevant volume of the deposited diluted blood sample into a layer of uniform thickness by bringing the two plates into the closed configuration;
(d) determining the amount of the calibration marker in a part or an entirety of the layer of thickness by detecting the calibration marker using the detector;
(e) estimating the volume of said part or entirety of the layer of thickness by timing the pre-determined uniform height of the spacers and the lateral area of said part or entirety of the layer of uniform thickness;
(f) obtaining the second concentration by dividing the determined amount of the calibration marker in step (d) by the estimated volume in step (e).

In the method of Embodiment 12 or any of its derived embodiments, the spacers regulating the layer of uniform thickness have a filling factor of at least 1%, the filling factor is the ratio of the spacer area in contact with the layer of uniform thickness to the total plate area in contact with the layer of uniform thickness.

In the method of Embodiment 12 or any of its derived embodiments, for spacers regulating the layer of uniform thickness, the Young's modulus of the spacers times the filling factor of the spacers is equal or larger than 10 MPa, the filling factor is the ratio of the spacer area in contact with the layer of uniform thickness to the total plate area in contact with the layer of uniform thickness.

In the method of Embodiment 12 or any of its derived embodiments, for a flexible plate, the thickness of the flexible plate times the Young's modulus of the flexible plate is in the range 60 to 750 GPa-um.

In the method of Embodiment 12 or any of its derived embodiments, for a flexible plate, the fourth power of the inter-spacer-distance (ISD) divided by the thickness of the flexible plate (h) and the Young's modulus (E) of the flexible plate, ISD4/(hE), is equal to or less than 106 um3/GPa.

In the method of Embodiment 12 or any of its derived embodiments, one or both plates comprises a location marker, either on a surface of or inside the plate, that provide information of a location of the plate.

In the method of Embodiment 12 or any of its derived embodiments, one or both plates comprises a Scale marker, either on a surface of or inside the plate, that provide information of a lateral dimension of a structure of the sample and/or the plate.

In the method of Embodiment 12 or any of its derived embodiments, one or both plates comprises an imaging marker, either on surface of or inside the plate, that assists an imaging of the sample.

In the method of Embodiment 12 or any of its derived embodiments, the spacers functions as a location marker, a scale marker, an imaging marker, or any combination of thereof.

In the method of Embodiment 12 or any of its derived embodiments, the average thickness of the layer of uniform thickness is in the range of 2 μm to 2.2 μm and the sample is blood.

In the method of Embodiment 12 or any of its derived embodiments, the average thickness of the layer of uniform thickness is in the range of 2.2 μm to 2.6 μm and the sample is blood.

In the method of Embodiment 12 or any of its derived embodiments, the average thickness of the layer of uniform thickness is in the range of 1.8 μm to 2 μm and the sample is blood.

In the method of Embodiment 12 or any of its derived embodiments, the average thickness of the layer of uniform thickness is in the range of 2.6 μm to 3.8 μm and the sample is blood.

In the method of Embodiment 12 or any of its derived embodiments, the average thickness of the layer of uniform thickness is in the range of 1.8 μm to 3.8 μm and the sample is whole blood without a dilution by another liquid.

In the method of Embodiment 12 or any of its derived embodiments, the average thickness of the layer of uniform thickness is about equal to a minimum dimension of an analyte in the sample.

In the method of Embodiment 12 or any of its derived embodiments, the inter-spacer distance is in the range of 7 μm to 50 μm.

In the method of Embodiment 12 or any of its derived embodiments, the inter-spacer distance is in the range of 50 μm to 120 μm.

In the method of Embodiment 12 or any of its derived embodiments, the inter-spacer distance is in the range of 120 μm to 200 μm.

In the method of Embodiment 12 or any of its derived embodiments, the inter-spacer distance is substantially periodic.

In the method of Embodiment 12 or any of its derived embodiments, the spacers are pillars with a cross-sectional shape selected from round, polygonal, circular, square, rectangular, oval, elliptical, or any combination of the same.

In the method of Embodiment 12 or any of its derived embodiments, the spacers are in pillar shape and have a substantially flat top surface, wherein, for each spacer, the ratio of the lateral dimension of the spacer to its height is at least 1.

In the method of Embodiment 12 or any of its derived embodiments, each spacer has the ratio of the lateral dimension of the spacer to its height is at least 1.

In the method of Embodiment 12 or any of its derived embodiments, the minimum lateral dimension of spacer is less than or substantially equal to the minimum dimension of an analyte in the Sample.

In the method of Embodiment 12 or any of its derived embodiments, the minimum lateral dimension of spacer is in the range of 0.5 μm to 100 μm.

In the method of Embodiment 12 or any of its derived embodiments, the minimum lateral dimension of spacer is in the range of 0.5 μm to 10 μm.

In the method of Embodiment 12 or any of its derived embodiments, the layer of uniform thickness sample is uniform over a lateral area that is at least 1 mm².

### 5 Device and Method for Composite Liquid Sample Separation

### 5.1 Device for Composite Liquid Sample Separation

In one aspect, the present invention also provides a device for separating a component from a composite liquid sample, comprising: a collection plate having a plurality of pillar spacers on one of its surfaces, and a filter having a sample receiving surface and a sample exit surface, wherein at least a part of the pillar spacers of the collection plate contact with and point against the sample exit surface, forming micro-cavities confined by the sample exit surface and said part of the pillar spacers, wherein the micro-cavities provide a capillary force that is at least a first part of a driving force for causing at least a part of the sample that is deposited on the sample receiving surface to flow through the filter toward the collection plate, and wherein the filter is configured to separate said component from said part of the sample.

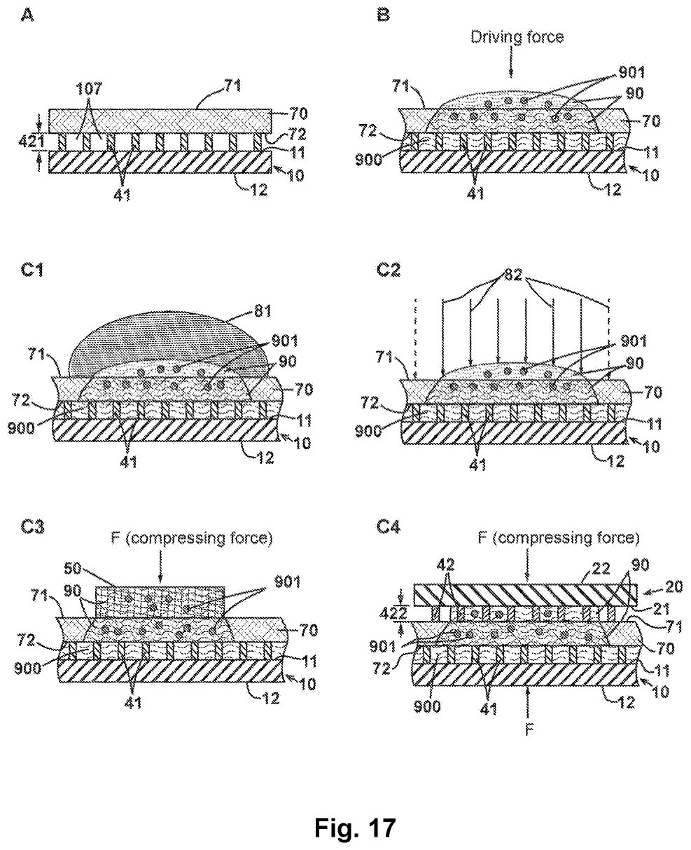

FIG. 17 panel (A) illustrates one exemplary embodiment of the device, where the device comprises a collection plate 10 and a filter 70. As shown in panel (A), in some embodiments, the collection plate 10 has an inner surface 11, an outer surface 12, and a plurality of pillar spacers 41 on its inner surface 11. The filter 70 has a sample receiving surface 71 and a sample exit surface 72. In some embodiments, the pillar spacers 41 are fixed on the inner surface 11. At least a part of the pillar spacers 41 point against and be in contact with the sample exit surface 72 of the filter 70, forming microcavities 107 that are confined by the sample exit surface 72 and said part of the pillar spacers 41.

FIG. 17 panel (B) further illustrates the exemplary embodiment of the device, where a composite liquid sample 90 containing a component 901 to be removed, is deposited on the sample receiving surface 71 of filter 70. According to the present invention, the filter 70 is configured to separate the component 901 from the part of the sample 90 as it flows through the filter 70 from the sample receiving surface 71 toward the collection plate 10. As shown in panel (B), in some embodiments, at least a part of the sample 90 is driven by a driving force to flow through the filter 70, in a direction from the sample receiving surface 71 toward the sample exit surface 72 and the collection plate 10. As the part of the sample 90 flows through the filter 70, the component 901 is retained and/or removed by the filter 70 from the filtering product 900—the part of the sample that exits the filter 70. In some embodiments, the microcavities 107 and/or the filter 70 provide a capillary force that is at least a part of the driving force. In some embodiments, the capillary force the microcavities 107 and/or the filter 70 provide is the only and the entire part of the driving force. However, in other embodiments, the capillary force from the microcavities 107 and/or the filter 70 is only a part of, sometimes even a negligible part of, the driving force.

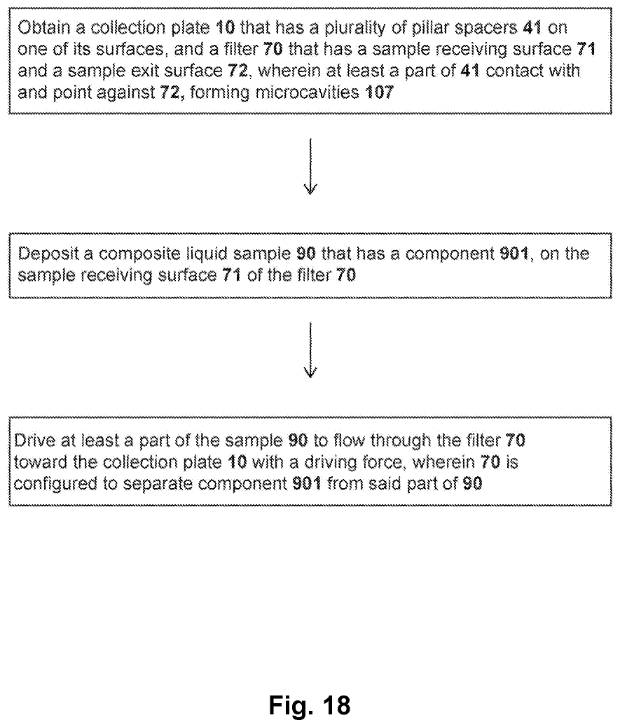

The features as stated for the common device, as shown in FIG. 17 panels (A) and (B) and described thereof, are also applicable to the embodiments shown in all the other panels in FIG. 17, FIGS. 18 to 20 and described thereof. In addition, it should be noted that the device serves as an example for the features shown in all figures and described thereof.

FIG. 17 panels (C1) to (C4) schematically show different embodiments of the device disclosed herein, where the device further comprises a source providing at least a part of the driving force for causing at least part of the sample 90 to flow through the filter 70 toward the collection plate 10. Different exemplary embodiments of such a source are illustrated from panel (C1) to panel (C4), respectively. These exemplary sources disclosed herein are by no means meant to be exclusive as to other possible embodiments and combination of any these sources with other embodiments. These sources disclosed herein are deployed separately, alternatively, sequentially or combinatorically, or in any other manner as long as it serves its main function, that is to provide at least a part of the driving force for causing the sample flow for the component separation by the filter 70.

As shown in FIG. 17 panel (C1), in some embodiments, the device further comprises a source (not shown) providing a first liquid 81 that has a low, if not zero, intermiscibility with the sample 90 and is configured to provide at least a part of the driving force. For instance, in situations where the sample 90 is a water-based solution, the first liquid 81 may be chosen from various types of hydrocarbon oils including, but not limited to, mineral oil, gasoline and related products, vegetable oils, and any mixture thereof. In some embodiments, the first liquid 81 has higher density than the sample 90 and it drives the sample flow out of its own gravity. In some embodiments, the first liquid 81 experiences a larger capillary force provided by the microcavities 107 and/or the filter 70 and consequently is capable of driving the sample 90 to flow. In other embodiments, the first liquid 81 is pressurized and the pressure is applied against the filter 70 and the collection plate 10, therefore forcing the sample 90 to flow toward the collection plate.

In yet other embodiments, the first liquid 81 has high intermiscibility with the sample 90, as long as it is configured to drive a part of the sample 90 to flow through the filter 70, for instance it can be highly pressurized. However, it should be noted that this type of configuration may compromise the quality of the filtering product 900, for instance, the filtering product 900 may be contaminated by the first liquid 81, and thus the analyte in the filtering product 900 may be diluted and/or altered physically or chemically by the contaminating first liquid 81, which may not be desirable in most applications.

As shown in FIG. 17 panel (C2), in some embodiments, the device further comprises a source (not shown) providing a pressured gas 82 that is configured to provide at least a part of the driving forces. As illustrated, in some embodiments, the pressured gas 82 is applied against at least part of the sample 90 in the direction from the sample receiving surface 71 toward the sample exit surface 72.

In some embodiments, the device further comprises a sponge for providing at least a part of the driving force. The term “sponge” as used herein, refers to refers to a flexible porous material that has pores with their shapes changeable under a force and that can absorb a liquid into the material or release a liquid out of the material, when the shape of the pores is changed. The sponge usually has an uncompressed state and a compressed state. Under the uncompressed state, the porous structure of the sponge reaches its maximum internal dimension, that is the internal pores are in their largest shape having their highest possible volume therein in the absent of major external influences, while under the compressed state, in some embodiments, the sponge experiences an external compressing force, and consequently, the internal pores of the sponge are compressed and deformed to a shape with dimensions smaller than the maximum internal dimension. The major external influences refer to any external impact that deforms the internal pores of the sponge. When a sponge deforms in a direction from its compressed state to the uncompressed state, the sponge can absorb any liquid it is in fluid connection with; when the sponge deforms in an opposite direction, from its uncompressed state to the compressed state, the sponge releases the liquid it contains therein.

For example, FIG. 17 panel (C3) illustrates some embodiments of the device, where the device further comprises a sponge 50. As aforementioned, the sponge 50 has an uncompressed state and a compressed state. In some embodiments, the sponge 50 is relatively movable to the collection plate and the filter into different configurations:
(i) one of the configurations is a depositing configuration (not shown), in which: the Sponge 50 is in the uncompressed state and separated, partially or completely, from the collection plate 10 and the filter 70, the distance between the collection plate 10 and the sponge 50 is not regulated by the spacers 41, the filter 70, or the deposited sample 90,
(ii) another of the configurations is a filtering configuration, in which: as shown in panel (C3), the filter 70 is positioned between the sponge 50 and the collection plate 10, the distance between the collection plate 10 and the sponge 50 is regulated by the spacers 41, the filter 70, and the deposited sample 90, the sponge 50 is in the compressed state, which is configured to provide at least a part of the driving force.

According to these embodiments, in the depositing configuration, the sponge 50 absorbs the liquid sample when placed in contact with the sample 90 so that a part or an entirety of the sample 90 enters the sponge 50 as shown in the figure. When the sponge 50, the collection plate 10, and the filter 70 are brought into their filtering configuration (i.e. the sponge 50 is compressed by a compressing force to its compressed state, and the distance between the collection plate 10 and the sponge 50 is regulated by the spacers 41, the filter 70, and the deposited sample 90), part of the absorbed sample 90 in the sponge 50 is forced to exit the sponge 50 and flow through the filter 70 toward the collection plate 10. Therefore, the component 901 is retained and/or removed from the filtering product 900. In some embodiments, the compressing force is applied on the sponge 50 in a direction against the filter 70. In other embodiments, the compressing force is applied on the sponge 50 in any other direction, so long as the sample 90 is forced to flow through the filter 70 toward the collection plate 10.

FIG. 17 panel (C4) shows yet other embodiments of the device, where the device further comprises a press plate 20, the press plate 20 having a plurality of spacers 42 on one of its surfaces. In some embodiments, the press plate 20 is relatively movable to the collection plate 10 and the filter 70 into different configurations:
(i) one of the configurations is a depositing configuration, in which the press plate 20 is separated, partially or completely, from the collection plate 10 and the filter 70, the distance between the collection plate 10 and the press plate 7 is not regulated by their spacers 41 and 42, the filter 70, or the deposited sample 90.
(ii) another of the configurations is a filtering configuration, in which: as shown in FIG. 1 panel (C4), the filter 70 is positioned between the press plate 20 and the collection plate 10, the distance between the collection plate 10 and the press plate 20 is regulated by their spacers 41 and 42, the filter 70, and the deposited sample 90, at least a part of the pillar spacers 42 and an inner surface 21 of the press plate press at least a part of the deposited sample 90 against the filter 70, providing at least a part of the driving force.

FIG. 17 panel (C4) shows that, in some embodiments, the collection plate 10, the filter 70, and the press plate 20 are brought into the filtering configuration by a compressing force that is applied over the press plate outer surface 22 and the collection plate outer surface 12. In the filtering configuration, the press plate pillar spacers 42 point against and are in contact with the filter 70 and at least part of the deposited sample 90. The distance between the press plate inner surface 11 and the sample receiving surface 71 is reduced to about the height of the pillar spacers 42. In some embodiments, in the filtering configuration of the device, at least a part of the deposited sample 90 is forced to flow through the filter 70 toward the collection plate 10, due to one of the following reasons, any combination thereof or any other possibilities: (a) the height of the pillar spacers 42 are configured to be smaller than the unconfined height of the deposited sample 90; (b) the filter 70 is configured to have a relatively low hindrance for the deposited sample 90 to flow through it in the direction from the sample receiving surface 71 toward the sample exit surface 72; (c) the microcavities 107 are configured to provide a relatively high capillary force to attract the sample flow toward the collection plate 10, and (d) the pillar spacer 42 are configured to provide a relatively high hindrance for the lateral flow of deposited sample 90.

### X-Plate

In some embodiments of the present invention, the collection plate is also termed “X-plate”. It is a plate that comprises, on its surface, ( ) spacers that have a predetermined inter-spacer distance and a predetermined height and are fixed on the surface, and (ii) a sample contact area for contacting a sample to be deposited, wherein at least one of the spacers is inside the sample contact area.

In some embodiments, the press plate is also a “X-plate”. Therefore, in these embodiments, the press plate, the filter, and the collection plate, in the filtering configuration of the device, become a sandwich-like structure, with the filter being compressed in the center by the two X-plates.

The details of the X-plates are pre-determined to provide appropriate parts of the driving force for causing the deposited sample to flow through the filter from the press plate side to the collection plate side, including, but not limited to, the thickness, shape and area, flexibility, surface flatness and wetting properties of the plate, the height, lateral dimension, interspace of the pillar spacers, the material and mechanical strength of the plate and pillar spacers.

In some embodiments, the X-plate includes, but not limited to, the embodiments described in U.S. Provisional Patent Application No. 62/202,989, which was filed on Aug. 10, 2015, U.S. Provisional Patent Application No. 62/218,455, which was filed on Sep. 14, 2015, U.S. Provisional Patent Application No. 62/293,188, which was filed on Feb. 9, 2016, U.S. Provisional Patent Application No. 62/305,123, which was filed on Mar. 8, 2016, U.S. Provisional Patent Application No. 62/369,181, which was filed on Jul. 31, 2016, U.S. Provisional Patent Application No. 62/394,753, which was filed on Sep. 15, 2016, PCT Application (designating U.S.) No. PCT/US2016/045437, which was filed on Aug. 10, 2016, PCT Application (designating U.S.) No. PCT/US2016/051775, which was filed on Sep. 14, 2016, PCT Application (designating U.S.) No. PCT/US2016/051794, which was filed on Sep. 15, 2016, and PCT Application (designating U.S.) No. PCT/US2016/054025, which was filed on Sep. 27, 2016, all of these disclosures are hereby incorporated by reference for their entirety and for all purposes.

### Filter

The term “filter”, as used herein, refers to a device that has at least a sample receiving surface and a sample exit surface, and that eliminates certain component from a composite liquid sample, when the liquid sample flows through the filter in a direction that traverses both the first and sample exit surfaces. According to the present invention, the filter can be a mechanical, chemical, or biological filter, or any combination thereof.

In some embodiments of the present invention, the filter can be a mechanical filter. Mechanical filter mechanically eliminates, trapping or blocking, certain solid components from a composite liquid sample when the sample flows through the filter in a certain direction. It is typically made of porous material, whereas the pore size determines the size of the solid particles capable of flowing through the filter and the size of the solid particle being eliminated from the sample that flows through it. The components of mechanical means are inert, so that they will not affect or interfere the sample. Examples of mechanical filter include, but not limited to, foam (reticulated and/or open Cell), fibrous material (e.g. filter paper), gel, sponge. Examples of materials include cellulose acetate, cellulose esters, nylon, polytetrafluoroethylene polyester, polyurethane, gelatin, agarose, polyvinyl alcohol, polysulfone, polyester sulfone, polyacrilonitrile, polyvinylidiene fluoride, polypropylene, polyethylene, polyvinyl chloride, polycarbonate, any other materials that can form porous structure and any combination thereof.

In some embodiments of the present invention, the pore size of the mechanical filter is uniform or vary in a range with a pre-determined distribution. In some embodiments, the average pore size of the mechanical filter is 10 nm, 20 nm, 40 nm, 80 nm, 100 nm, 200 nm, 400 nm, 800 nm, 1 μm, 2 μm, 4 μm, 8 μm, 10 μm, 20 μm, 40 μm, 80 μm, 100 μm, 500 μm, 1 mm to 1 cm, 5 mm, or a range between any of the values.

In some embodiments of the present invention, the filter is a chemical filter, which chemically eliminates certain components from a composite liquid sample when the sample flows through it in a certain direction. In some embodiments, it comprises a chemical reactant and a housing for the chemical reactant. The chemical reactant specifically reacts with certain component that is to be eliminated from the sample. It is capable of binding and immobilizing the component, or converting the component to other material(s) that is/are either retained in the housing or released outside of the housing and the filtering product. In some embodiments, the chemical reactant is inorganic chemical, organic chemical, or any combination thereof. In some embodiments, the chemical reactant is be biological material, including, but not limited to, antibody, oligonucleotide, other biological macromolecules that have affinity to the component that is to be eliminated from the sample.

In some embodiments of the present invention, the filter can also be a biological filter. Biological filter comprises a biological living matter and a housing for the living matter. In some embodiments, the living matter specifically ingests, engulfs, or binds to and immobilizes certain component in the sample. Exemplary living matters that can be used in the biological filter include, but not limited to, bacteria, fungus, virus, mammalian cells that have engulfing functions or affinity binding properties, like macrophage, T-cell, and B-cell.

### 5.2 Method for Composite Liquid Sample Separation

In one further aspect, the present invention provides a method for composite liquid sample separation, comprising the steps of

(1) providing a collection plate having a plurality of pillar spacers on one of its surfaces, and a filter having a sample receiving surface and a reverse sample exit surface, wherein at least a part of the pillar spacers of the collection plate are in contact with and point against the sample exit surface, forming micro-cavities confined by the sample exit surface and said part of the pillar spacers of the collection plate,
(2) depositing the sample on the sample receiving surface of the filter, and
(3) driving at least apart of the deposited sample to flow through the filter toward the collection plate with a driving force, wherein the filter is configured to separate said component from said part of the deposited sample, and wherein at least a first part of the driving force is a capillary force provided by the micro-cavities.

FIG. 18 is a flow chart for an exemplary embodiment of the method disclosed in the present invention. In this embodiment, the exemplary device as shown in FIG. 17 panel (A) is used.

First, a user of the device obtains a collection plate 10 having a plurality of pillar spacers 41 on one of its surfaces, and a filter 70 having a sample receiving surface 71 and a sample exit surface 72, wherein at least a part of the pillar spacers 41 contact with and point against the sample exit surface 72, forming microcavities 107, which are confined by the sample exit surface 72 and the collection plate 10. Next, depositing the composite liquid sample 90, having a component 901 to be separated from the sample, on the sample receiving surface 71 of the filter 70. After the depositing step, driving at least a part of the sample 90 to flow through the filter 70 toward the collection plate 10 with a driving force, wherein the filter 70 is configured to separate component 901 from said part of 90, resulting in the filtering product 900, and wherein the microcavities 107 are configured to provide a part of the driving force.

In some embodiments, the part of the driving force that the microcavities 107 provide is an entirety of the driving force. In these embodiments, the driving step is indeed to let the microcavities draw the part of sample 90 toward the collection plate 10 via capillary force, without any need of external influences.

In other embodiments, the part of the driving force that the microcavities 107 provide is only a part thereof, such that another source is needed to provide the other part of the driving force. For instance, in some embodiments, gravity participates in the process of driving the sample 90 to flow through the filter 70, when the sample receiving surface 71 is further from the earth compared to the sample exit surface 72 and the collection plate 10. Or in other cases, another source is part of the device as provided above, including, but not limited to, a source providing a first liquid 81, a source providing a pressured gas 82, a sponge 50, and a press plate 20. The driving force provided by these sources, as well as the gravity, may be exploited separately, alternatively, sequentially, or combinatorically, or in any other manners as long as to serve their main function, that is to provide at least a part of the driving force for causing the sample flow for the component separation by filter 70. According to these embodiments, the driving step of the method further comprises providing and operating the source for providing at least a part of the driving force.

In some embodiments, the driving step of the method comprises depositing a first liquid to contact the deposited sample, the first liquid having low intermiscibility with the sample and configured to provide at least a part of the driving force.

In other embodiments, the driving step of the method comprises applying a pressurized gas against the deposited Sample, the pressurized gas being configured to provide at least a part of the driving force.

In other embodiments, the driving step of the method comprises: (a) contacting a sponge with the deposited sample; (b) compressing the sponge against the filter to provide at least a part of the driving force.

In yet other embodiments, the driving step of the method comprises: (a) placing a press plate, having a plurality of pillar spacers on one of its surfaces, to contact with the deposited sample, wherein at least a part of the pillar spacers of the press plate point against the sample receiving surface of the filter and are in contact with the deposited sample; (b) after the placing step (a), compressing the press plate against the filter to reduce the distance between the press plate and the filter, and to provide at least a part of the driving force.

### 5.3. Sample

The composite liquid sample, according to the present invention, comprises one or more components to be separated by the devices and methods provided by the present invention from the sample.

The devices and methods herein disclosed is used for samples such as but not limited to diagnostic sample, clinical sample, environmental sample and foodstuff sample. The types of sample include but are not limited to the samples listed, described and summarized in PCT Application (designating U.S.) No. PCT/US2016/045437, which was filed on Aug. 10, 2016, and is hereby incorporated by reference by its entirety.

In particular embodiments, the sample is obtained from a biological sample such as cells, tissues, bodily fluids, and stool. Typically, samples that are not in liquid form are converted to liquid form before analyzing the sample with the present method. Bodily fluids of interest include but are not limited to, amniotic fluid, adueous humour, vitreous humour, blood (e.g., whole blood, fractionated blood, plasma, serum, etc.), breast milk, cerebrospinal fluid (CSF), cerumen (earwax), chyle, chime, endolymph, perilymph, feces, gastric acid, gastric juice, lymph, mucus (including nasal drainage and phlegm), pericardial fluid, peritoneal fluid, pleural fluid, pus, rheum, saliva, sebum (skin oil), semen, sputum, sweat, synovial fluid, tears, vomit, urine and exhaled condensate. In particular embodiments, a sample is obtained from a subject, e.g., a human. In some embodiments, it is processed prior to use in the subject assay. For example, prior to analysis, the protein/nucleic acid is extracted from a tissue sample prior to use, methods for which are known. In particular embodiments, the sample is a clinical Sample, e.g., a sample collected from a patient.

In particular embodiments, the sample is obtained from an environmental sample, including, but not limited to liquid samples from a river, lake, pond, ocean, glaciers, icebergs, rain, snow, sewage, reservoirs, tap water, drinking water, etc., solid samples from soil, compost, sand, rocks, concrete, wood, brick, sewage, etc., and gaseous samples from the air, underwater heat vents, industrial exhaust, vehicular exhaust, etc. Typically, samples that are not if liquid form are converted to liquid form before analyzing the sample with the present method. in particular embodiments, the sample is obtained from a food sample that is suitable for animal consumption, e.g., human consumption. A foodstuff sample includes, but not limited to, raw ingredients, cooked food, plant and anima sources of food, preprocessed food as well as partially or fully processed food, etc. Typically, samples that are not in liquid form are converted to liquid form before analyzing the sample with the present method.

According to the present invention, the component(s) to be separated from the sample can be in solid, liquid, gaseous state, or any combination thereof. The components to be Separate from the sample include, but not limited to, cells, tissues, virus, bacterium, protes, DNAs, RNAs, gas bubbles, lipids.

In a preferred embodiment of the present invention, the sample is a whole blood sample, and the components to be separated from the whole blood sample are blood cells (red blood cells, white blood cells, platelets, etc.). Thereby, if the preferred embodiment, the devices and methods are particularly configured for plasma separation.

According to the present invention, the sample volume is 1 μL of less, 2 μL of less, 5 μL or less, 10 μL or less, 20 μL or less, 50 μL or less, 100 μL or less, 200 μL or less, 1 mL of less, 2 mL or less, 5 mL or less, 10 mL or less, 20 mL or less, 50 mL or less, 100 mL or less, 200 mL or less, 500 mL or less, 1 L or less, or a rage between any of the values.

### 5.4 Filtering Product

In some embodiments of the present inversion, the collection plate is an X-plate, which, in addition to the composite sample separation, is used in a QMAX process for further sensing assays processing of the filtering product.

In the QMAX (Q: quantification; M: magnifying, A. adding reagents, X: acceleration; also known as compressed regulated open flow (CROF)) process or assay or assay platform, a QMAX device uses two plates to manipulate the shape of a sample into a thin layer (e.g. by compressing).

In QMAX assays, one of the plate configurations is an open configuration, wherein the two plates are completely or partially separated (the spacing between the plates is not controlled by spacers) and a sample can be deposited. Another configuration is a closed configuration, wherein at least part of the sample deposited in the open configuration is compressed by the two plates into a layer of highly uniform thickness, the uniform thickness of the layer is confined by the inner surfaces of the plates and is regulated by the plates and the spacers.

In some embodiments of the present invention, after filtering the sample, the fitter and the source providing the second part of the driving force are separated from the collection plate. The filtering product is retained on the collection plate, at east partially due to capillary force and surface tension. In some embodiments, the collection plate bearing the filtering product are joined with a capture plate to form a QMAX device: the collection pate aid the capture plate are relatively movable to each other into different configurations, wherein one of the configurations is an open configuration, in which the collection plate and the capture plate are separated apart, the spacing between the plates is not regulated by the spacers, wherein another of the configurations is a closed configuration, it which the plates are facing each other, the spacers and the filtering product are between the plates, the thickness of the filtering product is regulated by the plates and the spacers and is thinner than that when the plates are in the open configuration, and at least one of the spacers is inside the sample.

In some embodiments of the present invention, the capture pate is a planar glass pate, and/or comprises a birding site or a storage site that contains a binding agent of a detection agent, respectively, for an assay of the filtering product, in some embodiments, the collection plate also comprises a binding site or storage Site for at assay of the filtering product.

In some embodiments, the QMAX device that the collection plate and the capture plate form after the filtering process includes, but not limited to, the embodiments described in U.S. Provisional Patent Application No. 62/202,989, which was filed on Aug. 10, 2015, U.S. Provisional Patent Application No. 62/218,455, which was filed on Sep. 14, 2015, U.S. Provisional Patent Application No. 62/293,188, which was filed on Feb. 9, 2016, U.S. Provisional Patent Application No. 62/305,123, which was filed on Mar. 8, 2016, U.S. Provisional Patent Application No. 62/369,181, which was filed on Jul. 31, 2016, U.S. Provisional Patent Application No. 62/394,753, which was filed on Sep. 15, 2016, PCT Application (designating U.S.) No. PCT/US2016/045437, which was filed on Aug. 10, 2016, PCT Application (designating U.S.) No. PCT/US2016/051775, which was filed on Sep. 14, 2016, PCT Application (designating U.S.) No. PCT/US2016/051794, which was filed on Sep. 15, 2016, and PCT Application (designating U.S.) No. PCT/US2016/054025, which was filed on Sep. 27, 2016, all of these disclosures are hereby incorporated by reference for their entirety and for all purposes.

### 5.5 Advantageous Effects at Applications

The devices and methods provided by the present invention may find use in a variety of different applications in various fields, where separation of undesired components from a giver composite liquid sample and/or extraction of desired components from a given sample are feeded. For example, the subject device and method may find use in assays involving blood plasma where separation of blood cell is required, in applications requiring pure water without contaminating particles, in applications involving investigations of the contaminating bacterium in drinking water” and the like. Ëshe various fields include, but not limited to, human, veterinary, agriculture, foods, environments, drug testing, and others.

The devices and methods provided in the present invention have many advantages over existing art for composite liquid sample separation for manifold reasons, including, but not limited to: the devices and methods provided in some preferred embodiments can be relatively much simpler and easier to operate, void of the feed for well-trained professionals, require a much shorter time and a much lower cost, and, in some particular embodiments, are especially good at handling small volume of liquid sample.

In addition, the devices provided in some preferred embodiments of the present invention may be used to form a QMAX device, which may use in a wider range of applications. These applications include, but not limited to, biochemical assays, quantitative sampling of liquid sample, biochemical processing, and biomarker detections.

The devices and methods herein disclosed have various types of biological/chemical Sampling, Sensing, assays and applications, which include, but not limited to, those described in PCT Application (designating U.S.) No. PCT/US2016/045437, which was filed on Aug. 10, 2016, and PCT/US16/51794, which was filed on Sep. 14, 2016 are hereby incorporated by reference by its entirety.

The devices and methods herein disclosed are used for the detection, purification and/or quantification of analytes such as but not limited to biomarkers. Examples of the biomarks include but not be limited to what is listed, described and summarized in PCT Application (designating U.S.) No. PCT/US2016/045437, which was filed on Aug. 10, 2016, and is hereby incorporated by reference by its entirety.

The devices and methods herein disclosed are used With the facilitation and enhancement of mobile communication devices and systems, which include devices and systems listed, described and summarized in PCT Application (designating U.S.) No. PCT/US2016/045437, which was filed on Aug. 10, 2016, and is hereby incorporated by reference by its entirety.

### 5.6 Example 1

Here exemplary devices and methods for separating plasma from whole blood sample according to the present invention have been achieved experimentally. Experiments have been carried out to test and compare different experimental conditions for plasma separation.

For this experiment, two different types of X-plates were used as the collection plate according to the present invention. Both were made of PMMA and 175 μm thick and 1 inch by 1 inch wide. Type 1 X-plate has, on its surface, cubical pillar spacers of 30×40 μm in width and 30 μm in height and interspaced by 80 μm inter-spacing distance (ISD). Type 2 X-plate has, on its surface, cubical pillar spacers with all the same parameters as Type 1 except with 2 μm in height.

In some experimental conditions, a different X-plate, chosen from one of the two types, was used as the press plate as well. Four types of filter membranes (all purchased from Sterlitech Corp., Kent, Wash. and made of polycarbonate) with different pore sizes (0.4 μm, 1 μm, 2 μm, and 3 μm) were used as the filter for separating the blood cells from the plasma in the blood sample.

Whole blood sample was obtained either commercially or freshly by pricking a human subject's finger. As for all experimental conditions, during plasma separation, a filter membrane was set on top of a collection plate, which was placed on a bench with its pillar spacers pointing upward, and then a drop of whole blood sample (1 uL when using press plate with 2 um high spacers and 3 ul. When using planar glass plate, sponge, or press plate with 30 um high spacers) was deposited on top of the filter membrane for plasma separation. Either a planar glass plate, a sponge, or a press plate was used as the press media for providing the driving force for causing the blood sample to flow through the filter membrane toward the collection plate. The press media was placed on top of the deposited blood sample, and then hand-pressed against the collection plate for a certain amount of time (30 or 180 seconds), thereby forcing the blood sample to flow through the filter membrane for plasma separation.

After the hand-pressing for plasma separation, the top press media and the filter membrane were peeled off, while the filtering product stayed on the collection plate. A different planar glass plate (“capture plate”, 1 mm thick and 1 inch×1 inch wide) was then placed to contact the collection plate. Here, a QMAX process was then used for sample observation and quantitation. The collection plate and the capture plate were hand-pressed against each other for 30 second and then “self-held” to form a QMAX device. The resulting QMAX device bearing the filtered product was then imaged under light microscope, and the volume of the filtering product was estimated accordingly.

11 different experimental conditions have been tested in this experiment and the details of each Condition are summarized in Table 2.

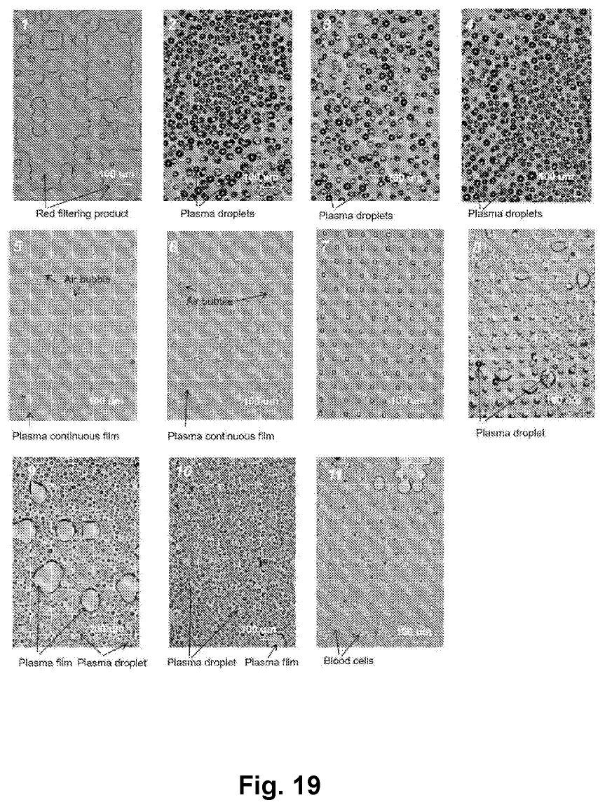

FIG. 19 shows the representative images of the filtering products resulted from different experimental configurations of the device when used for plasma separation. Number on the top left corner of each image denotes its experimental group number as listed in Table 2, and the periodically arranged rounded rectangles shown in each image are the pillar spacers of the Collection plates. As shown in the images, glass plates (Group 1) apparently lysed the red blood cells in the sample, leaving the filtering product in visible red color, group 11 showed blood cells in the filtering product, indicating that the pore size (5 um) was not small enough to filter out the blood cells, group 7 showed little plasma or blood, likely due to the oversize of sponge, which absorbed and retained most, if not all, the blood sample. Plasma was obtained in all the other groups: as seen from the images, groups 5 and 6 gave the best results as the filtering product (plasma) formed continuous films in the QMAX device, groups 2, 3, 4, 8, 9, and 10 showed mainly plasma droplets and occasionally a few patchy plasma films, likely due to the 30 um pillar height of the collection plate, as compared to the 2 um pillar height in groups 5 and 6.

TABLE 2
Experimental conditions

| | Experimental Condition | | | | |
|---|---|---|---|---|---|
| | | | Collection | | |
| | | | plate | | Blood |
| | Press media | Filter | pillar | Hand press | sample |
| Group | (pillar height) | pore size | height | duration | Volume |
| 1 | Glass | 0.4 μm | 30 μm | 30 s | 3 μL |
| 2 | X-Plate (30 μm) | 0.4 μm | 30 μm | 30 s | 3 μL |
| 3 | X-Plate (30 μm) | 0.4 μm | 30 μm | 180 s | 3 μL |
| 4 | X-Plate (2 μm) | 0.4 μm | 30 μm | 30 s | 1 μL |
| 5 | X-Plate (2 μm) | 0.4 μm | 2 μm | 30 s | 1 μL |
| 6 | X-Plate (30 μm) | 0.4 μm | 2 μm | 30 s | 3 μL |
| 7 | Sponge | 0.4 μm | 30 μm | 30 s | 3 μL |
| 8 | X-Plate (30 μm) | 1.0 μm | 30 μm | 30 s | 3 μL |
| 9 | X-Plate (30 μm) | 2.0 μm | 30 μm | 30 s | 3 μL |
| 10 | X-Plate (30 μm) | 3.0 μm | 30 μm | 30 s | 3 μL |
| 11 | X-Plate (30 μm) | 5.0 μm | 30 μm | 30 s | 3 μL |

An estimation of the filtering product volume was performed by timing the height of the pillar spacers by the summed area of plasma calculated from the image, and the filtering efficiency was calculated by dividing the volume of the filtering product by the volume of the whole blood sample. The overall data is summarized in Table 3.

TABLE 3
Filtering product quantitation

| | Results | |
|---|---|---|
| | | Efficiency |
| Group | Filtering product | (product/whole blood) |
| 1 | ~1 μL (with HbA) | ~30% |
| 2 | ~0.3 μL | ~10% |
| 3 | ~0.3 μL | ~10% |
| 4 | ~0.2 μL | ~20% |
| 5 | ~0.2 μL | ~20% |
| 6 | ~0.3 μL | ~10% |
| 7 | ~0.1 μL | ~3% |
| 8 | ~0.4 μL | ~13% |
| 9 | ~0.5 μL | ~17% |
| 10 | ~0.5 μL | ~17% |
| 11 | N/A | N/A |

This example illustrates the validity of the devices and methods provided by the present invention. It also demonstrates the advantages of using the present invention to realize plasma separation: the exemplary devices have relatively much simpler structure and are much easier to handle, as compared to many other existing arts in the field; the method takes much shorter time, likely within 1 min from obtaining the device and sample to the complete of the plasma separation; the method is capable of handling very small amount of blood sample, reducing the burden on subjects, especially patients, by avoiding the invasive drawing of large amount of blood.

### 5.7 Example-2

Here, the plasma separated by the exemplary device and method as illustrated in Example-1 has been demonstrated to be used for a triglyceride (TG) assay, a part of a regular labtest. TGs are a type of fat found in the blood, high level of TGs may raise the risk of coronary artery disease. Therefore, TG test is a part of a lipid panel that is used to evaluate an individual's risk of developing heart disease. Typically, TG assay is a colorimetric assay and performed with plasma instead of whole blood sample to avoid color interference from hemoglobins in red blood cells. An exemplary device and method were used here to separate plasma from a whole blood sample, and the resulting plasma was used as a substrate for the TG assay.

In this experiment, for plasma separation, an X-plate (PMMA, 175 μm thick and 1 inch by 1 inch wide, cubical pillar spacers: 30×40 μm wide, 30 μm high, and 80 μm ISD) was used as the collection plate. Filter membrane with 0.4 μm pores (Sterlitech Corp., Kent, Wash.) was used as the filter. A different X-plate (PMMA, 175 μm thick and 1 inch by 1 inch wide, cubical pillar spacers: 30×40 μm wide, 30 μm high, and 80 μm ISD) was used as the press plate. About 2 ul whole blood sample was obtained freshly by pricking a subject's finger and deposited on the filter membrane, which was placed on top of the pillar spacers of the collection plate, and then the press plate was placed on top of the deposited sample and hand-pressed against the collection plate for 30 S. Part of the whole blood sample was thereby forced to flow through the filter membrane toward the collection plate, realizing plasma separation.

For the TG assay, after plasma separation, the filter membrane and the press plate were then peeled off from the collection plate, leaving plasma—the filtering product—on the collection plate. Next, 0.5 μL TG assay reagent (Express Biotech International Inc., Frederick, Md.) was deposited on a capture plate (a planar plastic plate, made of PMMA with 1 mm thick and 3 inch by 1 inch wide) and then transferred onto the plasma on the collection plate. The capture plate was hand-pressed against the collection plate, forming a QMAX device, to incubate the TG assay for 1 min. The assay image was then read by an iPhone, which was pre-configured to capture and analyze images from QMAX devices.

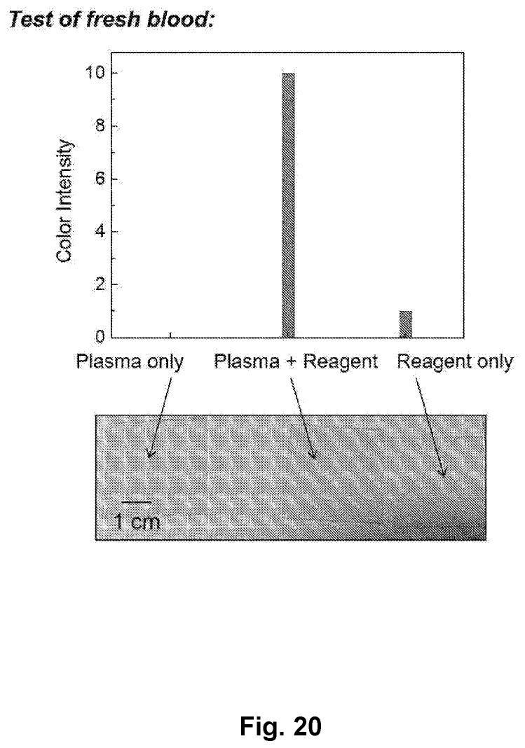

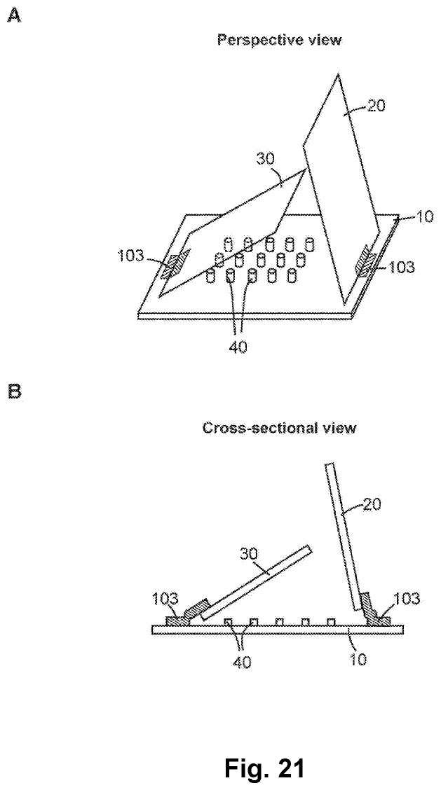

FIG. 20 shows the results of a triglyceride (TG) assay using the filtering products from the experimental filtering device as the assay sample and the QMAX device as the assay device. The bottom panel shows the picture of the QMAX devices used for TG assay and imaging. As shown, a long planar glass plate was used to Contact and pressed against all three Collection plates that were tested, forming three separate QMAX devices. The TG assay here is a colorimetric assay, in that the assay solution changes color (turn to pink) when detecting TG and a higher color intensity indicates a higher level of TG in the assay sample. The top panel shows a graph plot of the color intensity results under three different experimental conditions. The color intensity was close to zero when there was plasma (filtering produce) only, and at a very low level when there was reagent only. However, the color intensity reached the highest level when the plasma and reagent were both present, indicating the existence of TGs in the plasma.

The example illustrates again the validity of the devices and methods provided by the present invention. It also clearly demonstrates the ease of combining the present invention with QMAX process, which would significantly accelerate the sampling/sensing/assay/processing of the sample and expand the applicability of QMAX devices.

### 6 Summary of Embodiments for Separating Composite Liquid Sample

The present invention includes a variety of embodiments, which can be combined in multiple ways as long as the various components do not contradict one another. The embodiments should be regarded as a single invention file: each filing has other filing as the references and is also referenced in its entirety and for all purpose, rather than as a discrete independent. These embodiments include not only the disclosures in the current file, but also the documents that are herein referenced, incorporated, or to which priority is claimed.

### 6.1 A Device for Separating a Component from a Composite Liquid Sample

Embodiment 13: A device for separating a component from a composite liquid sample, comprising:

a collection plate having a plurality of spacers that are fixed on one of its surfaces, and a filter having a sample receiving surface and a sample exit surface,
wherein at least a part of the spacers point against and are in contact with the sample exit surface of the filter, forming microcavities confined by the sample exit surface and said part of the spacers, and
wherein the filter is configured to separate said component from a part of the sample that flows through the filter from the sample receiving surface toward the collection plate.

In the device of Embodiment 13, the microcavities provide a capillary force that constitutes at least a part of a driving force for causing at least a part of the sample that is deposited on the sample receiving surface to flow through the filter toward the collection plate.

In the device of Embodiment 13 or any of its derived embodiments, the device further comprises a force source providing a first liquid that is configured to provide at least a part of the driving force, the first liquid has low intermiscibility with the sample.

In the device of Embodiment 13 or any of its derived embodiments, further comprising a force source providing a pressurized gas that is configured to provide at least a part of the driving force.

In the device of Embodiment 13 or any of its derived embodiments, the device further comprises a sponge,

wherein the sponge has a compressed state and an uncompressed sate,
wherein the sponge is movable relative to the collection plate and the filter into different configurations,
wherein one of the configurations is a depositing configuration, in which: the sponge is in the uncompressed state and separated, partially or completely, from the collection plate and the filter, the distance between the collection plate and the sponge is not regulated by the spacers, the filter, or the deposited sample, and
wherein another of the configurations is a filtering configuration, in which: the filter is positioned between the sponge and the collection plate, the distance between the collection plate and the sponge is regulated by the spacers, the filter, and the deposited sample, and the sponge is being converted from the uncompressed state to the compressed state, during which the sponge is configured to provide at least a part of the driving force.

In the device of Embodiment 13 or any of its derived embodiments, the device further comprises a press plate having a plurality of spacers on one of its surfaces,

wherein the press plate is relatively movable to the collection plate and the filter into different configurations,
wherein one of the configurations is a depositing configuration, in which the press plate is separated, partially or completely, from the collection plate and the filter, the distance between the collection plate and the press plate is not regulated by their spacers, the filter, or the deposited sample, and
wherein another of the configurations is a filtering configuration, in which: the filter is positioned between the press plate and the collection plate, the distance between the collection plate and the press plate is regulated by their spacers, the filter, and the deposited sample, and at least a part of the spacers and an inner surface of the press plate press at least a part of the deposited Sample against the filter, providing at least a part of the driving force.

In the device of Embodiment 13 or any of its derived embodiments, the press plate spacers have a uniform height in the range of 0.5 to 100 μm and a constant inter-spacer distance is in the range of 5 to 200 μm.

In the device of Embodiment 13 or any of its derived embodiments, the press plate spacers have a uniform height in the range of 1 to 50 μm and a constant inter-spacer distance is in the range of 7 to 50 μm.

### 6.2 A Method of Separating a Component from a Composite Liquid Sample

Embodiment 14: A method of separating a component from a composite liquid sample, comprising the steps of:

(1) providing a collection plate having a plurality of spacers on one of its surfaces, and a filter that has a sample receiving surface and a sample exit surface, wherein at least a part of the spacers point against and are in contact with the sample exit surface of the filter, forming microcavities confined by the sample exit surface and said part of the spacers,
(2) depositing the sample on the sample receiving surface of the filter, and
(3) driving at least a part of the deposited sample with a driving force to flow through the filter toward the collection plate, wherein the filter is configured to separate said component from said part of the deposited sample that flows through the filter from the sample receiving surface toward the collection plate.

In the method of Embodiment 14, the microcavities provide a capillary force that constitutes at least a part of the driving force in step (3).

In the method of Embodiment 14 or any of its derived embodiments, step (3) comprises depositing a first liquid to contact the deposited sample, the first liquid having low intermiscibility with the sample and configured to provide at least a part of the driving force.

In the method of Embodiment 14 or any of its derived embodiments, step (3) comprises applying a pressurized gas against the deposited sample, the pressurized gas being configured to provide at least a part of the driving force.

In the method of Embodiment 14 or any of its derived embodiments, step (3) comprises:

(a) contacting a sponge with the deposited sample, and

(b) compressing the sponge against the filter to provide at least a part of the driving force.

In the method of Embodiment 14 or any of its derived embodiments, step (3) comprises:

(a) placing a press plate having a plurality of spacers on one of its surfaces, to contact with the deposited sample, at least a part of the spacers of the press plate point against the sample receiving surface of the filter and are in contact with the deposited sample;

(b) after the placing step (a), compressing the press plate against the filter to reduce the distance between the press plate and the filter, and to provide at least a part of the driving force.

In the method of Embodiment 14 or any of its derived embodiments, the press plate spacers have a uniform height in the range of 0.5 to 100 μm and a constant inter-spacer distance is in the range of 5 to 200 μm.

In the method of Embodiment 14 or any of its derived embodiments, the press plate spacers have a uniform height in the range of 1 to 50 μm and a constant inter-spacer distance is in the range of 7 to 50 μm.

In the method of Embodiment 14 or any of its derived embodiments, the compressing step is performed by human hand.

### 6.3 The Device or Method of Separating a Component from a Composite Liquid Sample

Embodiment 15: The device or method of any one of prior embodiments, wherein the collection plate spacers have a predetermined substantially uniform height and a predetermined substantially constant inter-spacer distance.

In the device or method of Embodiment 15, the uniform height is in the range of 0.5 to 100 μm and the constant inter-spacer distance is in the range of 5 to 200 μm.

In the device or method of Embodiment 15, the uniform height is in the range of 0.5 to 20 μm and the constant inter-spacer distance is in the range of 7 to 50 μm.

In the device or method of Embodiment 15 or any of its derived embodiments, the filter is a mechanical filter, a chemical filter, a biological filter, or any combination thereof.

In the device or method of Embodiment 15 or any of its derived embodiments, the filter is made of a material selected from a group consisting of silver, glass fiber, ceramic, cellulose acetate, cellulose esters, nylon, polytetrafluoroethylene polyester, polyurethane, gelatin, agarose, polyvinyl alcohol, polysulfone, polyester sulfone, polyacrilonitrile, polyvinylidiene fluoride, polypropylene, polyethylene, polyvinyl chloride, polycarbonate, any other materials that can form porous structure and any combinations thereof.

In the device or method of Embodiment 15 or any of its derived embodiments, the filter has an average pore size in the range of 10 nm to 500 μm.

In the device or method of Embodiment 15 or any of its derived embodiments, the filter has an average pore size in the range of 0.1 to 5 μm.

### 6.4 A Device for Plasma Extraction from a Blood Sample

Embodiment 16: A device for plasma extraction from a blood sample, comprising:

a collection plate having a plurality of spacers that are fixed on one of its surfaces, and a filter having a sample receiving surface and a sample exit surface,
wherein at least a part of the spacers point against and are in contact with the sample exit surface of the filter, forming microcavities confined by the sample exit surface and said part of the spacers;
wherein the spacers have a uniform height in the range of 1 to 50 μm and constant inter spacer distance in the range of 7 to 50 μm; and
wherein the filter is configured to separate blood cells from a part of the blood sample that flows through the filter from the sample receiving surface toward the collection plate, and made of a material selected from a group consisting of silver, glass fiber, ceramic, cellulose acetate, cellulose esters, nylon, polytetrafluoroethylene polyester, polyurethane, gelatin, agarose, polyvinyl alcohol, polysulfone, polyester sulfone, polyacrilonitrile, polyvinylidiene fluoride, polypropylene, polyethylene, polyvinyl chloride, polycarbonate, any other materials that can form porous structure and any combinations thereof, and has an average pore size in the range of 0.1 to 5 μM.

In the device of Embodiment 16, the microcavities provide a capillary force that consists at least a part of a driving force for causing at least a part of a sample that is deposited on the sample receiving surface to flow through the filter toward the collection plate.

### 6.5 A Method of Plasma Extraction from a Blood Sample

Embodiment 17: A method of plasma extraction from a blood sample, comprising the steps of:

(1) providing a collection plate having a plurality of spacers on one of its surfaces, and a filter having a sample receiving surface and a sample exit surface,
wherein at least a part of the spacers point against and are in contact with the sample exit surface of the filter, forming microcavities confined by the sample exit surface and said part of the spacers, and
wherein the spacers have a uniform height in the range of 1 to 50 μm and a constant inter-spacer distance in the range of 7 to 50 μm;
(2) depositing the blood sample on the sample receiving surface of the filter, and
(3) driving at least a part of the deposited blood sample with a driving force to flow through the filter toward the collection plate,
wherein the filter is configured to separate blood cells from said part of the deposited blood sample that flows through the filter from the sample receiving surface toward the collection plate, and made of a material selected from a group consisting of: silver, glass fiber, ceramic, cellulose acetate, cellulose esters, nylon, polytetrafluoroethylene polyester, polyurethane, gelatin, agarose, polyvinyl alcohol, polysulfone, polyester sulfone, polyacrilonitrile, polyvinylidiene fluoride, polypropylene, polyethylene, polyvinyl chloride, polycarbonate, any other materials that can form porous structure and any combinations thereof, and has an average pore size in the range of 0.1 to 5 μm.

In the method of Embodiment 17, the microcavities provide a capillary force that consists at least a part of the driving force in step (3).

In the method of Embodiment 17 or any of its derived embodiments, the depositing step comprises: (a) pricking the skin of a human release a droplet of blood onto the skin, and (b) contacting the droplet of blood with the filter without use of a blood transfer tool.

### 6.6 A Device for Plasma Separation from a Blood Sample

Embodiment 18: A device for plasma separation from a blood sample, comprising:

a collection plate and a press plate, both of which have a plurality of spacers that are fixed on one of its surfaces, and a filter having a sample receiving surface and a sample exit surface,
wherein at least a part of the collection plate spacers point against and are in contact with the sample exit surface of the filter, forming microcavities confined by the sample exit surface and said part of the spacers,
wherein the spacers of the collection plate and the press plate have a uniform height in a range of 1 to 50 μm and a constant inter-spacer distance in the range of 7 to 50 μm, respectively;
wherein the filter is configured to separate blood cells from a part of the blood sample that flows through the filter from the sample receiving surface toward the collection plate, and made of a material selected from a group consisting of silver, glass fiber, ceramic, cellulose acetate, cellulose esters, nylon, polytetrafluoroethylene polyester, polyurethane, gelatin, agarose, polyvinyl alcohol, polysulfone, polyester Sulfone, polyacrilonitrile, polyvinylidiene fluoride, polypropylene, polyethylene, polyvinyl chloride, polycarbonate, any other materials that can form porous structure and any combinations thereof, and has an average pore size in the range of 0.1 to 5 μm;
wherein the press plate is relatively movable to the collection plate and the filter into different configurations,
wherein one of the configurations is a depositing configuration, in which the press plate is separated, partially or completely, from the collection plate and the filter, the distance between the collection plate and the press plate is not regulated by their spacers, the filter, or the deposited sample, and
wherein another of the configurations is a filtering configuration, in which: the filter is positioned between the press plate and the collection plate, the distance between the collection plate and the press plate is regulated by their spacers, the filter, and the deposited sample, at least a part of the spacers and an inner surface of the press plate press at least a part of the deposited sample against the filter, providing at least a part of the driving force.

### 6.7 A Method of Plasma Extraction from a Blood Sample

Embodiment 19: A method of plasma extraction from a blood sample, comprising the steps of:

(1) providing a collection plate and a press plate, both of which have a plurality of spacers on one of its surfaces, and a filter having a sample receiving surface and a sample exit surface,
wherein at least a part of the collection plate spacers point against and are in contact with the sample exit surface of the filter, forming microcavities confined by the sample exit surface and said part of the spacers, and
wherein the spacers of the collection plate and the press plate have a uniform height in a range of 1 to 50 μm and a constant inter-spacer distance in the range of 7 to 50 μm, respectively;
(2) depositing the blood sample on the sample receiving surface of the filter,
(3) placing a press plate having a plurality of spacers on one of its surfaces, to contact with the deposited blood sample, wherein at least a part of the spacers of the press plate point against the sample receiving surface of the filter and are in contact with the deposited sample, and
(4) after the placing step, compressing the press plate against the filter to reduce the distance between the press plate and the filter, and to force at least a part of the deposited blood sample to flow through the filter toward the collection plate,
wherein the filter is configured to separate blood cells from said part of the deposited blood sample that flows through the filter from the sample receiving surface toward the collection plate, and made of a material selected from a group consisting of: silver, glass fiber, ceramic, cellulose acetate, cellulose esters, nylon, polytetrafluoroethylene polyester, polyurethane, gelatin, agarose, polyvinyl alcohol, polysulfone, polyester sulfone, polyacrilonitrile, polyvinylidiene fluoride, polypropylene, polyethylene, polyvinyl chloride, polycarbonate, any other materials that can form porous structure and any combinations thereof, and has an average pore size in the range of 0.1 to 5 μm.

In the method of Embodiment 19, the compressing step is performed by human hand.

In the method of Embodiment 19 or any of its derived embodiments, the depositing step comprises: (a) pricking the skin of a human release a droplet of blood onto the skin, and (b) contacting the droplet of blood with the filter without use of a blood transfer tool.

### 6.8 The Device or Method of Plasma Extraction from a Blood Sample

Embodiment 20: The device or method of any one of prior embodiments, wherein each of the plates has a thickness of less than 200 μm.

In the method of Embodiment 20, each of the plates has a thickness of less than 100 μm.

In the method of Embodiment 20 or any of its derived embodiments, each of the plates has an area of less than 5 cm².

In the method of Embodiment 20 or any of its derived embodiments, each of the plates has an area of less than 2 cm².

In the method of Embodiment 20 or any of its derived embodiments, at least one of the plates is made from a flexible polymer.

In the method of Embodiment 20 or any of its derived embodiments, at least one of the plates is a flexible plate, and the thickness of the flexible plate times the Young's modulus of the flexible plate is in the range of 60 to 75 GPa-um.

In the method of Embodiment 20 or any of its derived embodiments, the spaces are fixed on the inner surface of the second plate.

In the method of Embodiment 20 or any of its derived embodiments, the spacers are pillars with a cross sectional shape selected from round, polygonal, circular, square, rectangular, oval, elliptical, or any combination of the same.

In the method of Embodiment 20 or any of its derived embodiments, the spacers have a pillar shape and a substantially flat top surface, wherein, for each spacer, the ratio of the lateral dimension of the spacer to its height is at least 1.

In the method of Embodiment 20 or any of its derived embodiments, each spacer has the ratio of the lateral dimension of the spacer to its height is at least 1.

In the method of Embodiment 20 or any of its derived embodiments, the minimum lateral dimension of spacer is less than or substantially equal to the minimum dimension of an analyte in the sample.

In the method of Embodiment 20 or any of its derived embodiments, the spacers have a pillar shape, and the sidewall corners of the spacers have a round shape with a radius of curvature at least 1 μm.

In the method of Embodiment 20 or any of its derived embodiments, the spacers have a density of at least 100/mm².

In the method of Embodiment 20 or any of its derived embodiments, the spacers have a density of at least 1000/mm².

In the method of Embodiment 20 or any of its derived embodiments, the spacers have a filling factor of at least 1%, the filling factor is the ratio of the spacer area in contact with the layer of uniform thickness to the total plate area in contact with the layer of uniform thickness.

In the method of Embodiment 20 or any of its derived embodiments, the Young's modulus of the spacers times the filling factor of the spacers is equal or larger than 10 MPa, the filling factor is the ratio of the spacer area in contact with the layer of uniform thickness to the total plate area in contact with the layer of uniform thickness.

In the method of Embodiment 20 or any of its derived embodiments, at least one of the plates is flexible, and for the flexible plate, the fourth power of the inter-spacer-distance (ISD) divided by the thickness of the flexible plate (h) and the Young's modulus (E) of the flexible plate, ISD/(hE), is equal to or less than 106 μm/GPa.

In the method of Embodiment 20 or any of its derived embodiments, the spacers are fixed on a plate by directly embossing the plate or injection molding of the plate.

In the method of Embodiment 20 or any of its derived embodiments, the materials of the plate and the spacers are independently selected from polystyrene, PMMG, PC, COC, COP, or another plastic.

### 7 Multi-Plate QMAX Device with Hinges and Filters

FIG. 21 shows an embodiment of a QMAX (Q: Quantification; M: magnifying, A. adding reagents, X: acceleration; also known as compressed regulated open flow (CROF)) device, which comprises a first plate 10, a second plate 20, a third plate 30 and spacer 40. Panel (A) shows the perspective view of the plates in an open configuration, in which: the plates are partially or entirely separated apart, the spacing between the plates are not regulated by the spacers 40, allowing a sample to be deposited on the one or more of the plates or one a structure, e.g. filter, this is placed on top of one of the plates, panel (B) shows the sectional view of the plates at the open configuration.

As shown in panels (A) and (B) of FIG. 21, in some embodiments the second plate 20 and the third plate 30 are both connected to the first plate 10. In certain embodiments, the second plate 20 is connected to the first plate 10 with a hinge 103, the third plate 30 is connected to the first plate 10 with another hinge 103. The second plate 20 and the third plate 30 are configured such that each can pivot toward and away from the first plate 10 without interfering with each other. In some embodiments, the surface of the first plate 10 facing the second plate 20 and the third plate 30 is defined as the inner surface, the surfaces of the second plate 20 and the third plate 30 that face the first plate 10 are also defined as the inner surfaces of the respective plates.

In some embodiments, the hinges 103 are partly placed on top of the inner surface of the first plate 10 and connect the second plate 20 and the third plate 30 to the first plate 10. In certain embodiments, the edges of the second plate 20 and/or the edges of the third plate 30 are not closely aligned with the edge of the first plate 10. In certain embodiments, the hinges 103 do not wrap around any edge of the first plate 10. It should also be noted, however, that the second plate 20 and the third plate 30 are not required to be connected to the first plate 10. In certain embodiments, the second plate 20 and/or the third plate 30 are completely separated from the first plate 10.

In some embodiments, the hinges are configured that one or more hinges can be torn off to make the plates become unconnected. In some embodiments, one plate is teared off before a closing of the other two plates. In some embodiments, the plates are not connected by hinges.

Panels (A) and (B) of FIG. 21 also show spacers 40, which are fixed on the first plate 10. It should also be noted, however, that the spacers 40 can be fixed on the third plate 30, the second plate 20 or any selections and combinations of the three plates. In certain embodiments, the spacers 40 are fixed on the inner surfaces of the first plate 10 and the third plate 30. In certain embodiments, the spacers 40 are fixed on the inner surfaces of the first plate 10 and the second plate 20. In certain embodiments, the spacers 40 are fixed on the inner surfaces of the second plate 20 and the third plate 30. In certain embodiments, the spacers 40 are fixed only on the first plate 10. In certain embodiments, the spacers 40 are fixed only on the second plate 20. In certain embodiments, the spacers 40 are fixed only on the third plate 30. In certain embodiments, the spacers 40 are fixed on all three plates. When the spacers 40 are fixed on more than one plate, the spacer heights on the different plates can be the same or different. In some embodiments, the spacers 40 are not fixed on any plate but are mixed in the sample.

It should be noted that in some embodiments, the spacers 40 are not a required structure. In certain embodiments, none of the plates comprises spacers that are fixed on the plates or added in the samples.

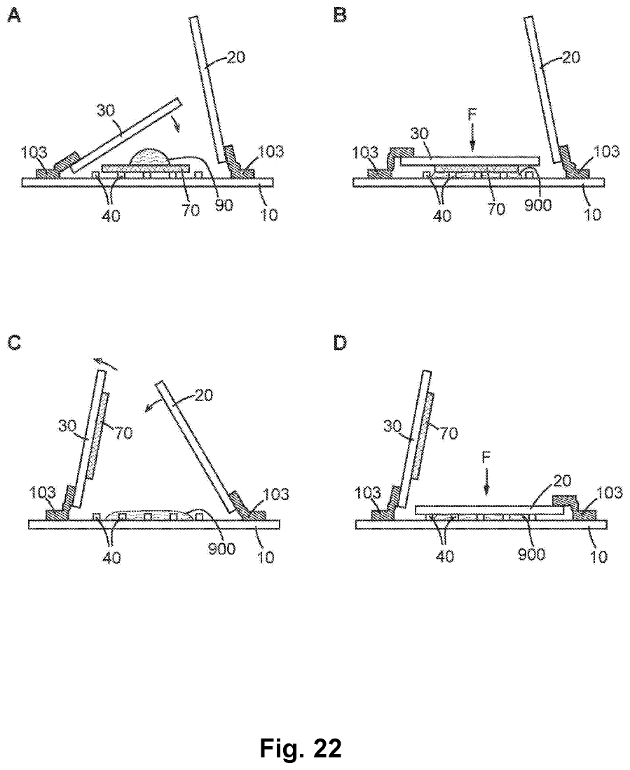

FIG. 22 shows an exemplary embodiment of the QMAX device and the process to utilize the QMAX device to filter and analyze a liquid sample. In certain embodiments, the elements as shown in FIG. 22 are organized into the kit. For example, in certain embodiments the kit comprises a QMAX device and a filter, wherein the QMAX device comprises a first plate 10, a second plate 20, a third plate 30 and spacers 40, wherein the second plate 20 and the third plate 30 are connected to the first plate 10, e.g. with hinges 103.

Panel (A) of FIG. 22 shows the sectional view of a QMAX device in an open configuration, where a sample 90 is deposited on a filter 70, which is placed on top of the first plate 10. As shown in panel (A), in certain embodiments the filter 70 is actually placed on top of the spacers 40 so that a cavity is left between the filter 70 and the inner surface of the first plate 10. In some embodiments, the sample 70 is placed on top of the filter, wherein the sample comprises multiple components. In certain embodiments, the sample comprises at least one component that can be separated by the filter from the rest of the sample, in certain embodiments, the component of the sample is blocked or absorbed by the filter 70 and separated from the part of the sample 90 that flows through the filter 70 and into the cavity. In some embodiments, the sample 90 is whole blood. In certain embodiments, the component of the sample 90 that is blocked or absorbed by the filter 70 comprises the blood cells; the part of the sample 90 that flows through the filter 70 comprises the plasma.

The components as shown in panel (A) of FIG. 22 can be elements of a kit, which comprises a first plate 10, a second plate 20, a third plate 30, spacers 40, and filter 70, wherein the second plate 20 and the third plate 30 are connected to the first plate 10 so that the second plate 20 and the third plate 30 can pivot toward and away from the first plate 10. As shown in panel (A), in certain embodiments the second plate 20 and the third plate 30 are connected to the first plate 10 with hinges 103. In some embodiments, the kit of the present invention further comprises a wash pad and washing solution, wherein the wash pad the washing solution can be used to wash the inner surface of the first plate 10 after depositing sample 90 on the first plate 10. In certain embodiments, the washing can be conducted after certain components in the sample 90 can be incubated after the second plate 20 has been pressed against the first plate 10 for a certain period of time.

Panel (B) of FIG. 22 shows the sectional view of a QMAX device when the third plate is pressed on top of the filter, pushing part of the sample to flow through the filter. In some embodiments, the filter covers all the spacers 40. In some embodiments, the filter only covers part of the spacers 40. As shown in panels (A) and (B), after the sample 90 is deposited on the top of the filter 70, the third plate 30 can be pressed toward the filter, making the third plate 30 essentially parallel to the first plate 10 so that part of the sample 90 flows through the filter 70, when one or more components of the sample 90 are trapped or absorbed in the filter 70. As shown in panel (B), the part of the sample 90 that flows through the filter 70 can be referenced as the filtered sample 900. In certain embodiments, part of the sample 90 flows through the filter 70 due to capillary force in the filter 70 and the capillary force in the cavity formed between the filter 70 and the first plate 10.

In some embodiments, the spacers 40 are fixed only on the first plate 10, not the third plate 30. In some embodiments, the spacers 40 are fixed only on the third plate 30, not the first plate 10. In some embodiments, the spacers 40 are fixed on both the first plate 10 and the third plate 30. In certain embodiments, when the spacers 40 are fixed on the third plate 30, using the third plate 30 to press against the filter 70 can prevent damaging certain components of the sample 90. For example, in certain embodiments, when the sample 90 is whole blood, pressing the sample 90 with the third plate 30 that has spacers 40 can prevent lysing some cells (e.g. red blood cells) in the blood. In some embodiments, the lysing of the cells is not desirable at least partly because the elements in the cells can be released into the plasma and flows through the filter 70, causing confusion to the analysis results. It should also be noted that, in certain embodiments, when the properties of the spacers 40 are properly selected, there can be not lysing or damaging of any components of the sample 90.

In some embodiments of the present invention, the filter can be a mechanical filter. Mechanical filter mechanically eliminates, absorbs, traps or blocks certain components from a composite liquid sample when the sample flows through the filter in a certain direction. It is typically made of porous material, whereas the pore size determines the size of the solid particles capable of flowing through the filter and the size of the solid particle being eliminated from the sample that flows through it. The components of mechanical means are inert, so that they will not affect or interfere the sample. Examples of mechanical filter include, but not limited to, foam (reticulated and/or open cell), fibrous material (e.g. filter paper), gel, sponge. Examples of materials include cellulose acetate, cellulose esters, nylon, polytetrafluoroethylene polyester, polyurethane, gelatin, agarose, polyvinyl alcohol, polysulfone, polyester sulfone, polyacrilonitrile, polyvinylidiene fluoride, polypropylene, polyethylene, polyvinyl chloride, polycarbonate, any other materials that can form porous structure and any combination thereof.

Panel (C) of FIG. 22 shows a sectional view of the QMAX device when the third plate 30 is opened after filtering and before the second plate 20 is pivoting towards the first plate 10. As shown in panels (B) and (C), after the pressing the sample 90 with the third plate 30, part of the sample 90—filtered sample 900—flows through the filter 70 and into the cavity between the filter 70 and the first plate 10. In some embodiments, after the filtering is complete or after a predetermined period of time, the third plate 30 and the filter 70 are opened so that the second plate 20 can be used. In some embodiments, the filter 70 is stuck to the third plate 30, either though the capillary effects or other mechanisms, can the combined filter 70 and the third plate 30 can be removed from the first plate 10 with one manipulating motion. In some embodiments, the filter 70 is not attached to the third plate 30; in certain embodiments, a user can open the third plate 30 first, and then remove the filter 70 from the first plate 10.

After opening the third plate 30 and the filter 70, the filtered sample 900 is left on the first plate 10. In some embodiments, when there are spacers 40 fixed on the first plate 10, the filtered sample 900 is positioned over and/or between the spacers 40. In certain embodiments, the second plate 20 can be pressed towards the second plate 20. In certain embodiments, there are no spacers 40 on the second plate 20; in certain embodiments, there are spacers 40 on the second plate 20.

Panel (D) of FIG. 22 shows a sectional view of the QMAX device in a closed configuration when the part of the sample (filtered sample 900) that flows through the filter 70 is pressed into a layer of uniform thickness by the second plate 20. As indicated, the plates are movable relative to one another into different configurations. One of the configuration between the second plate 20 and the first plate 10 is a closed configuration, in which: the first plate 10 and the second plate 20 are pressed together, the spacing between the second plate 20 and the first plate 10 is regulated by the height of the spacers 40; and at least part of the filtered sample 900 is pressed into a layer of uniform thickness. In certain embodiments, an external force F is used to pressed the first plate 10 and the second plate 20 together. In certain embodiments, after the removal of the force, the plates 10 and 20 can be kept at the closed configuration and the spacing between the plates are well maintained. In some embodiments, the spacing between the plates, the thickness of the layer of the filtered sample, and the height of the spacers 40 are the same.

After the first plate 10 and the second plate 20 are switched into a closed configuration, analysis and measurements can be carried out for the filtered sample 900 in the layer of uniform thickness. In Some embodiments, the thickness is less than 0.2 μm, 0.5 μm, 1 μm, 1.5 μm, 2 μm, 3 μm, 5 μm, 10 μm, 20 μm, 30 μm, 50 μm, 100 μm, 150 μm, 200 μm, 250 μm, 375 μm, or 500 μm, or in a range between any of the two values. In some embodiments, due the uniformity and the limited thickness of the filtered sample, the measurement and analysis can be carried out accurately and rapidly.

In certain embodiments, the sample is blood. After filtering with the filter 70, blood cells such as red blood cells and white blood cells are trapped, absorbed or blocked by the filter 70. The filtered sample 900 comprises blood plasma. In some embodiments, the blood plasma can be analyzed with various types of biological and/or chemical assays. For example, the glucose level in the plasma can be analyzed with colorimetric assays.

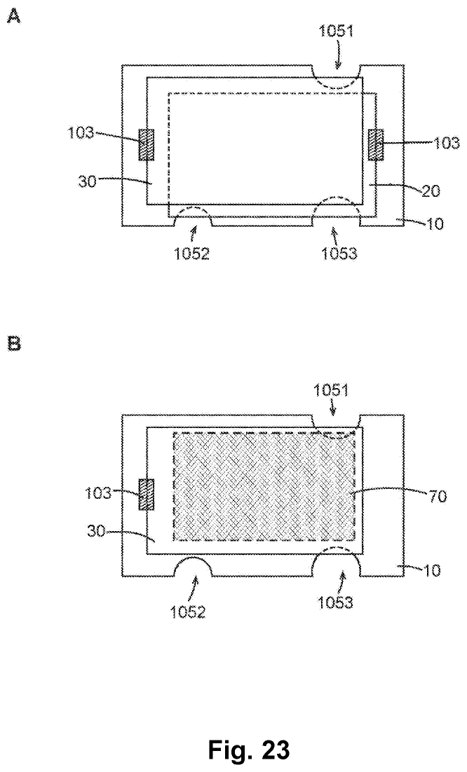

FIG. 23 shows an exemplary embodiment of the QMAX device. Panel (A) shows the top view of a QMAX device that comprises notches. Panel (B) shows the top view of a QMAX device that comprises notches when the filter 70 is placed on top of the first plate 10—for clarity purposes the second plate 20 is not shown in panel (B). In some embodiments, it would be convenient and/or necessary to include structures that facilitate pivoting of the second plate 20 and the third plate 30. In other words, in some embodiments, it would be convenient and/or necessary to include structures so that a user can adjust the angle between the first plate 10 the second plate 20, the angle between the first plate 10 and the third plate 30, and the positioning of the filter 70 relative to the first plate 10 and the third plate 30. FIG. 23 provides an example of such structures.

As shown in panels (A) and (B) of FIG. 23, the first plate 10 comprises a first notch 1051, a second notch 1052, and a third notch 1053. It should be noted that in certain embodiments the first plate 10 can comprise only one of the three notches, in certain embodiments the first plate 10 can comprise only two—any two—of the three notches.

In some embodiments, the sizes of these notches are the same. In some embodiments, the sizes of these notches are different. The sizes of the notches are adjusted according to the size of the plates and the specific needs of the user. For example, in some embodiments, the length of a notch, which is defined as the length of the widest opening on the notched edge, is less than 1 mm, 2.5 mm, 5 mm, 10 mm, 15 mm, 20 mm, 25 mm, 30 mm, 40 mm, 50 mm, or in a range between any of the two values. In some embodiments, the length of the notch is less than 1/10, 1/9, 1/7, ⅙, ⅕, ¼, ⅓, ⅖, ½, ⅗, ⅔, ¾, ⅘, ⅚, or 9/10 of the length of the notched edge, or in a range between any of the two values. In some embodiments, when the notch is in the shape of part of a circle, such a circle has a radius of less than 1 mm, 2.5 mm, 5 mm, 10 mm, 15 mm, 20 mm, 25 mm, 30 mm, 40 mm, 50 mm, or in a range between any of the two values.

FIG. 23 shows notches with a semicircle shape. However, it should be noted that the notches can be any shape as long as an opening is provided in the first plate 10 beneath the second plate 2 to facilitate opening the first plate 1 and second plate 2. For example, in some embodiments the notches have a shape of any part of a circle. In some embodiments, the notches have the shape of part or all of a square, rectangle, triangle, hexagon, polygon, trapezoid, sector-shape or any combinations of thereof. The notches on the same plate can have the same or different shapes.

As shown in panel (A) of FIG. 23, the first plate 10 comprises a first notch 1051, which is positioned and sized so that while one edge of the third plate 30 is partly juxtaposed over the first notch 1051, no edge of the second plate 20 is juxtaposed over it. In certain embodiments, the first notch 1051 is positioned to the far end-relative to the hinge 103 of the third plate 30 on the first plate 10. Conversely, the first notch 1051 can be positioned on the third plate 30, instead of the first plate 10, so that one edge of the first plate 10 is juxtaposed over the first notch 1051 and facilitate the manipulation of the relative positioning between the first plate 10 the third plate 30.

As shown in panels (A) and (B) of FIG. 23, in some embodiments, the first plate 10 comprises a first notch 1051 and a third notch 1053. In certain embodiments, when the filter 70 is positioned on top of the first plate 10, one edge of the filter 70 is juxtaposed over the first notch 1051, but not the second notch 1053. In certain embodiments, the third plate 30 is juxtaposed over both the first notch 1051 and the second notch 1053. With such a design, when a user wants to manipulate the position (e.g. change from a closed position to an open position) of the third plate 30 and the filter 70 together, the user can push the third plate 30 and the filter 70 above the first notch 1051, when a user wants to manipulate the position of only the third plate 30, the user can push the third plate 30 above the third notch 1053. It should also be noted that in some embodiments, the first plate 10 only comprises the first notch 1051, not the third notch 1053; the edges of the third plate 30 and the filter 70 over the first notch 1051 do not completely overlap, the user can choose to manipulate either the third plate 30 alone or the third plate 30 and the filter 70 together by change the placement of the force fore manipulation. In certain embodiments, the third notch 1053 is positioned to the far end—relative to the hinge 103 of the third plate 30—on the first plate 10. In addition, as the positioning of the first notch 1051, it would be possible to position the third notch 1053 on the third plate 30, not the first plate 10.

As shown in panel (A) of FIG. 23, in some embodiments, the first plate 10 comprises a second notch 1052. In certain embodiments, the second notch 1052 is positioned to the far end—relative to the hinge 103 of the second plate 20—on the first plate 10. In some embodiments, one edge of the second plate 20, but no edge of the first plate 10, is juxtaposed over the second notch 1052, facilitating changing the relative positioning of the second plate 20 and the first plate 10. Conversely, in certain embodiments the second notch 1052 is placed on the second plate 20, not the first plate 10.

Besides notches, other structures can also be used to facilitate the manipulation of the first plate 10, the second plate 20, the third plate 30 and the filter 70. For example, in some embodiments, any one, or two, or all three of the plates comprise tabs that are attached to the bodies of the plates. A user can manipulate the positioning of the plates by pulling the tabs.

For example, in some embodiments the second plate 20 comprises a plate tab, which is configured to facilitate switching the plates among different configurations between the second plate 20 and the second plate 20. In certain embodiments, the third plate 30 comprises a pressing tab which is configured to facilitate switching the plates among different configurations between the third plate 30 and the first plate 10. In addition, in some embodiments the filter 70 also comprises a tab. For example, in certain embodiments the filter 70 comprises a filter tab, which is configured to facilitate removing the filter from the plates.

### 8 Summary of Embodiments for Multi-Plate QMAX Device with Hinges and Filters

Further examples of inventive subject matter according to the present disclosure are described in the following enumerated paragraphs.

### 8.1 A Assay Method Using a Multi-Plate QMAX Device

### Use Two Plates for Transferring Reagent

Embodiment 21: A method for performing an assay, comprising

(a) obtaining a first plate comprising, on its inner surface, a sample contact area that has a first reagent site, wherein the first reagent site comprises an immobilized first reagent,
(b) obtaining a second plate comprising, on its inner surface, a sample contact area that has a storage site, wherein the storage site comprises an agent that is capable of, upon contacting a transferring liquid, diffusing in the transferring liquid, wherein the second agent binds to or reacts with the first agent,
wherein the first and second plates are movable relative to each other into different configurations, including an open configuration and a closed configuration;
(c) depositing the transferring liquid onto one or both of the sample contact areas of the plates in the open configuration,
(d) after (c), bringing the two plates to the closed configuration;
wherein in the open configuration the sample contact areas of the two plates are separated larger than 200 μm;
wherein, in the closed configuration, at least part of the transfer liquid deposited in (c) is confined between the sample contact areas of the two plates, and has an average thickness in the range of 0.01 to 200 μm.

### Use Three Plates

Embodiment 22: A method for performing an assay, comprising:

(a) obtaining a first plate comprising, on its inner surface, a Sample Contact area that has a first reagent site, wherein the first reagent site comprises a first reagent that bio/chemically interacts with a target analyte in a sample,
(b) obtaining a second plate comprising, on its inner surface, a sample contact area that has a second reagent site, wherein the second reagent site comprises a second reagent, that is capable of, upon Contacting the sample, diffusing in the sample,
(c) obtaining a third plate comprising, on its inner surface, a sample contact area that has a third reagent site, wherein the third reagent site comprises a third regent, that is capable of, upon contacting a transfer liquid, diffusing in the transfer liquid,
(d) depositing, in an open configuration, the sample on one or both of the sample contact areas of the first and second plates,
(e) after (d), bringing the first and second plates to a closed configuration;
(f) after (e) separating the first and second plate,
(g) after (f) depositing, in an open configuration, a transfer liquid on one or both of the sample contact areas of the second and third plates,
(h) after g), bringing the second and third plates to a closed configuration; and
(i) detecting a signal related to the target analyte,
wherein the first, second, and third plates are movable relative to each other into different configurations, including an open and a closed configurations,
wherein in the open configuration, the sample contact areas of the two plates are separated larger than 200 μm;
wherein, in the closed configuration, at least part of the sample deposited in (d) or the transfer liquid deposited in (g) is confined between the sample contact areas of the two plates, and has an average thickness in the range of 0.01 to 200 μm.

### 8.2 A Multi-Plate QMAX Device

Embodiment 23: A device for performing an assay, comprising:

a first plate comprises, on its inner surface, a sample contact area that has a first reagent site, wherein the first reagent site comprises a first reagent that bio/chemically interacts with a target analyte in a sample,
a second plate comprising, on its inner surface, a sample contact area that has a second reagent site, wherein the second reagent site comprises a second regent, that is capable of, upon contacting the sample, diffusing in the sample,
a third plate comprising, on its inner surface, a sample contact area that has a third reagent site, wherein the third reagent site comprises a third regent, that is capable of, upon contacting a transfer liquid, diffusing in the transfer liquid,
wherein the first, second, and third plates are movable relative to each other into different configurations, including an open and a closed configuration,
wherein in the open configuration, the sample contact areas of the two plates are separated larger than 200 μm;
wherein, in the closed configuration, at least part of the sample or the transfer liquid is confined between the sample contact areas of the two plates, and has an average thickness in the range of 0.01 to 200 μm;
wherein the sample is deposited on one or both of the sample contact areas of the first and second plates in the open configuration; and
wherein the transferring liquid is deposited on one or both of the sample contact areas of the second and third plates the open configuration.

### 8.3 A Multi-Plate QMAX Device and an Assay Method Thereof

Embodiment 24: The method or device of any prior embodiment, wherein one or both of the sample contact areas comprise spacers, wherein the spacers regulate the spacing between the sample contact areas of the plates when the plates are in the closed configuration,

In the method or device of Embodiment 24, the spacing between the sample contact areas when the plates are in a closed configuration is regulated by spacers.

In the method or device of Embodiment 24 or any of its derived embodiments, the device further comprises spacers that regulate the spacing between the sample contact areas when the plates are in a closed configuration.

In the method or device of Embodiment 24 or any of its derived embodiments, the storage site further comprises another reagent, in addition to the competitive agent.

In the method or device of Embodiment 24 or any of its derived embodiments, the binding site comprises, in addition to immobilized capture agent, another reagent that is, upon contacting the sample, capable of diffusion in the sample,

In the method or device of Embodiment 24 or any of its derived embodiments, the binding site faces the storage site when the plates are in the closed configuration.

In the method or device of Embodiment 24 or any of its derived embodiments, the first plate comprises a plurality of binding sites and the Second plate comprises a plurality of corresponding storage sites, wherein each biding site faces a corresponding storage site when the plates are in the closed configuration.

In the method or device of Embodiment 24 or any of its derived embodiments, the detection agent is dried on the storage site.

In the method or device of Embodiment 24 or any of its derived embodiments, the capture agents at the binding site are on an amplification surface that amplifies an optical signal of the analytes or the captured competitive agents in the embodiment 1, 2 and 3.

In the method or device of Embodiment 24 or any of its derived embodiments, the capture agents at the binding site are on an amplification surface that amplifies an optical signal of the analytes or the captured competitive agents in the embodiment 1, 2 and 3, wherein the amplification is proximity-dependent in that the amplification significantly reduced as the distance between the capture agents and the analytes or the competitive agents increases.

In the method or device of Embodiment 24 or any of its derived embodiments, the detection of the signal is electrical, optical, Fluorescence, SPR, etc.

In the method or device of Embodiment 24 or any of its derived embodiments, the sample is a blood sample (whole blood, plasma, or serum).

In the method or device of Embodiment 24 or any of its derived embodiments, the material of fluorescent microsphere is dielectric (e.g. Si02, Polystyrene,) or the combination of dielectric materials thereof.

In the method or device of Embodiment 24 or any of its derived embodiments, the method further comprises steps of adding the detection agent of said fluorescence label to the first plate to bind competitive agent.

In the method or device of Embodiment 24 or any of its derived embodiments, the method further comprises steps of washing after the detection agent is added to the first plate.

### 8.4 A Device for Sample Analysis

Embodiment 25: A device for sample analysis, comprising:

a first plate, a second plate, a third plate, and spacers, wherein:
i. the second plate and the third plate are respectively connected to the first plate, wherein the second plate and the third plate are configured to each pivot against the first plate without interfering with each other,
ii. by pivoting against the first plate, either the second plate or the third plate is movable relative to the first plate into different configurations
iii. the first plate comprises an inner surface that has a sample contact area for contacting a liquid sample that contains a component, and
iv. the spacers are fixed on one or more of the plates or are mixed in the sample, and
   wherein one of the configurations is an open configuration, in which: all three plates are partially or entirely separated apart and the spacing between the plates is not regulated by the spacers, and the sample is deposited on the first plate, the second plate, or both; and
   wherein another of the configurations is a closed configuration which is configured after the sample deposition in the open configuration, and in the closed configuration: at least part of the sample deposited is compressed by the first plate and the second plate into a layer of highly uniform thickness, which is confined by the inner surfaces of the first and second plates and is regulated by the plates and the spacers.

In the device of Embodiment 25, the device further comprises a filter made of a porous material.

In the device of Embodiment 25 or any of its derived embodiments, the filter is configured to separate said component from a part of the sample that flows through the filter.

In the device of Embodiment 25 or any of its derived embodiments, the third plate is configured to press the sample against the filter when the third plate pivots toward the first plate.

In the device of Embodiment 25 or any of its derived embodiments, one edge of the second plate is connected to the inner surface of the first plate with a first hinge.

In the device of Embodiment 25 or any of its derived embodiments, one edge of the third plate is connected to the inner surface of the first plate with a second hinge.

In the device of Embodiment 25 or any of its derived embodiments, one edge of the second plate is connected to the inner surface of the first plate with a first hinge, and one edge of the third plate is connected to the inner surface of the first plate with a second hinge.

In the device of Embodiment 25 or any of its derived embodiments, in the closed configuration between the first plate and second plate, the third plate can be adjusted to pivot against the first plate and the second plate.

In the device of Embodiment 25 or any of its derived embodiments, the first plate comprises one or more notches on one or more of its edges, wherein the notches are positioned such that the second plate and/or the third plate are juxtaposed on the notches to facilitate the manipulation of pivoting of the second plate and the third plate.

In the device of Embodiment 25 or any of its derived embodiments, the second plate comprises a plate tab, which is configured to facilitate switching the plates between different configurations.

In the device of Embodiment 25 or any of its derived embodiments, the filter comprises a filter tab, which is configured to facilitate removing the filter from the plates.

In the device of Embodiment 25 or any of its derived embodiments, the spacers are fixed on the first plate.

In the device of Embodiment 25 or any of its derived embodiments, the spacers are fixed on both the first and second plates.

In the device of Embodiment 25 or any of its derived embodiments, the sample is whole blood and the component is blood cells.

### 8.5 A Kit for Sample Washing and Analysis

Embodiment 26: A kit for sample washing and analysis, comprising:

a first plate, a second plate, a third plate, spacers and a filter, wherein:
i. the second plate and the third plate are respectively connected to the first plate, wherein the second plate and the third plate are configured to each pivot against the first plate without interfering with each other,
ii. by pivoting against the first plate, either the second plate or the third plate is movable relative to the first plate into different configurations,
iii. the first plate comprises an inner surface that has a sample contact area for contacting a liquid sample that contains a component, and
iv. the spacers are fixed on one or more of the plates or are mixed in the sample,
   wherein one of the configurations is an open configuration, in which: the three plates are partially or Completely separated apart, the spacing between the plates is not regulated by the spacers, allowing a liquid sample to be deposited on the first plate, the second plate, or both;
   wherein another of the configurations is a closed configuration which is configured after the sample deposition in the open configuration, and in the closed configuration: at least part of the sample deposited is compressed by the first plate and the second plate into a layer of highly uniform thickness, which is confined by the inner surfaces of the first and second plates and is regulated by the plates and the spacers, and
   wherein the filter is made of a porous material and configured to separate a component from a part of the sample that flows through the filter.

In the kit of Embodiment 26, the filter is configured to be pressed by the third plate when the filter is positioned on the first plate.

In the kit of Embodiment 26:
i. the sample comprises an analyte,
ii. a capture agent is coated on a sample contact area in the first plate, and
iii. the capture agent is configured to specifically bind to the analyte.

In the kit of Embodiment 26 or any of its derived embodiments, the filter is made of a material selected from a group consisting of silver, glass fiber, ceramic, cellulose acetate, cellulose esters, nylon, polytetrafluoroethylene polyester, polyurethane, gelatin, agarose, polyvinyl alcohol, polysulfone, polyester sulfone, polyacrilonitrile, polyvinylidiene fluoride, polypropylene, polyethylene, polyvinyl chloride, polycarbonate, any other materials that can form porous structure and any combinations thereof.

In the kit of Embodiment 26 or any of its derived embodiments, one edge of the second plate is connected to the inner surface of the first plate with a first hinge.

In the kit of Embodiment 26 or any of its derived embodiments, one edge of the third plate is connected to the inner surface of the first plate with a second hinge.

In the kit of Embodiment 26 or any of its derived embodiments, one edge of the second plate is connected to the inner surface of the first plate with a first hinge, and one edge of the third plate is connected to the inner surface of the first plate with a second hinge.

In the kit of Embodiment 26 or any of its derived embodiments, in the closed configuration between the first plate and second plate, the third plate can be adjusted to pivot against the first plate and the second plate.

In the kit of Embodiment 26 or any of its derived embodiments, the first plate comprises one or more notches on one or more of its edges, wherein the notches are positioned such that the second plate and/or the third plate are juxtaposed on the notches to facilitate the manipulation of pivoting of the second plate and the third plate.

In the kit of Embodiment 26 or any of its derived embodiments, the second plate comprises a plate tab, which is configured to facilitate switching the plates between different configurations.

In the kit of Embodiment 26 or any of its derived embodiments, the filter comprises a filter tab, which is configured to facilitate removing the filter from the plates.

In the kit of Embodiment 26 or any of its derived embodiments, the spacers are fixed on the first plate.

In the kit of Embodiment 26 or any of its derived embodiments, the spacers are fixed on both the first and second plates.

In the kit of Embodiment 26 or any of its derived embodiments, the sample is whole blood and the component is blood cells.

### 8.6 A Method of Analyzing a Component in a Sample

Embodiment 27: A method of analyzing a component in a sample, comprising:

(a) obtaining a sample that comprises a Component,
(b) obtaining a device comprising a first plate, a second plate, a third plate, a filter and spacers, wherein:
i. the second plate and the third plate are respectively connected to the first plate, wherein the second plate and the third plate are configured to each pivot against the first plate without interfering with each other,
ii. by pivoting against the first plate, either the second plate or the third plate is movable relative to the first plate into different configurations,
iii. the first plate comprises an inner surface that has a sample contact area for contacting a liquid sample that contains a component,
iv. the spacers are fixed on one or more of the plates or are mixed in the sample, and
v. the filter is placed on top of the first plate,
   (c) depositing the sample on top of the filter,
   (d) pressing the third plate against the sample and force a part of the sample to flow through the filter onto the first plate, wherein the filter is configured to separate the component from the part of the sample that flows through the filter,
   (e) removing the third plate and the filter from the first plate, and
   (f) compressing the part of the sample that flow onto the first plate into a layer of uniform thickness by pressing the first plate and second plate together.

In the method of Embodiment 27, the second plate and the third plate are respectively connected to the first plate, wherein the second plate and the third plate are configured to each pivot against the first plate without interfering with each other.

In the method of Embodiment 27 or any of its derived embodiments, one edge of the second plate is connected to the inner surface of the first plate with a first hinge.

In the method of Embodiment 27 or any of its derived embodiments, one edge of the third plate is connected to the inner surface of the first plate with a second hinge.

In the method of Embodiment 27 or any of its derived embodiments, one edge of the second plate is connected to the inner surface of the first plate with a first hinge, and one edge of the third plate is connected to the inner surface of the first plate with a second hinge.

In the method of Embodiment 27 or any of its derived embodiments, the first plate comprises one or more notches on one or more of its edges, wherein the notches are positioned such that the second plate and/or the third plate are juxtaposed on the notches to facilitate the manipulation of pivoting of the second plate and the third plate.

In the method of Embodiment 27 or any of its derived embodiments, the second plate comprises a plate tab, which is configured to facilitate switching the plates between different configurations.

In the method of Embodiment 27 or any of its derived embodiments, the filter comprises a filter tab, which is configured to facilitate removing the filter from the plates.

In the method of Embodiment 27 or any of its derived embodiments, the first plate comprises at least one assay site, wherein the sample deposited on the assay site and the spacers are fixed to the assay site.

In the method of Embodiment 27 or any of its derived embodiments, the first plate comprises a capture reagent coated on the inner surface of the first plate, wherein the capture reagent is configured to bind specifically to an analyte in the sample.

In the method of Embodiment 27 or any of its derived embodiments, the first plate comprises a plurality of assay sites spaced apart a minimum site spacing.

In the method of Embodiment 27 or any of its derived embodiments, the second plate contacts the sample with the inner surface of the second plate, and the inner surface of the second plate includes detection agents adhered, wherein the detection agents are configured to specifically associate at least one of the analyte and the analyte bound to the capture agent.

In the method of Embodiment 27 or any of its derived embodiments, the spacers are fixed on the first plate.

In the method of Embodiment 27 or any of its derived embodiments, the spacers are fixed on both the first and second plates.

In the method of Embodiment 27 or any of its derived embodiments, the sample is whole blood and the component is blood cells.

In the method of Embodiment 27 or any of its derived embodiments, the filter includes filter spacers on the wash surface, wherein the wash surface and the filter spacers are configured to prevent the direct contact between the wash surface and the assay site.

In the method of Embodiment 27 or any of its derived embodiments, the method further comprises: after the step (f), detecting the analyte bound to the capture agents.

In the method of Embodiment 27 or any of its derived embodiments, the detecting includes measuring at least one of fluorescence, luminescence, scattering, reflection, absorbance, and surface plasmon resonance associated with the analyte bound to the capture agents.

In the method of Embodiment 27 or any of its derived embodiments, the inner surface of the first plate at the assay site includes a signal amplification surface Such as a metal and/or dielectric microstructure (e.g., a disk-Coupled dots-On-pillar antenna array).

In the method of Embodiment 27 or any of its derived embodiments, the uniform thickness is at most 1 mm, at most 800 μm, at most 600 μm, at most 500 μm, at most 400 μm, at most 200 μm, at most 150 μm, at most 100 μm, at most 75 μm, at most 50 μm, at most 20 μm, at most 10 μm, or at most 2 μm, or in a range between any of the two values.

In the method of Embodiment 27 or any of its derived embodiments, the biological sample does not include spacers.

### 9 Additional Features

### 9.1 Q-Card, Spacer and Uniform Sample Thickness

The devices, systems, and methods herein disclosed can include or use Q-cards, spacers, and uniform sample thickness embodiments for sample detection, analysis, and quantification. In some embodiments, the Q-card comprises spacers, which help to render at least part of the sample into a layer of high uniformity. The structure, material, function, variation and dimension of the spacers, as well as the uniformity of the spacers and the sample layer, are herein disclosed, or listed, described, and summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, all of which applications are incorporated herein in their entireties for all purposes.

### 9.2 Other Embodiments

### (1) Dimensions

The devices, apparatus, systems, and methods herein disclosed can include or use a QMAX device, which can comprise plates and spacers. In some embodiments, the dimension of the individual components of the QMAX device and its adaptor are listed, described and/or summarized in PCT Application (designating U.S.) No. PCT/US2016/045437 filed on Aug. 10, 2016, and U.S Provisional Application Nos. 62,431,639 filed on Dec. 9, 2016 and 62/456,287 filed on Feb. 8, 2017, which are all hereby incorporated by reference by their entireties.

In some embodiments, the dimensions are listed in the Tables below:

### Plates:

| Para- | | |
|---|---|---|
| meters | Embodiments | Preferred Embodiments |
| Shape | round, ellipse, rectangle, triangle, polygonal, ring- | at least one of the two (or |
| | shaped, or any superposition of these shapes; the two | more) plates of the QMAX |
| | (or more) plates of the QMAX card can have the | card has round corners for |
| | same size and/or shape, or different size and/or shape; | user safety concerns, wherein |
| | | the round corners have a |
| | | diameter of 100 um or less, |
| | | 200 um or less, 500 um or less, |
| | | 1 mm or less, 2 mm or less, |
| | | 5 mm or less, 10 mm or less, |
| | | 50 mm or less, or in a range |
| | | between any two of the |
| | | values. |
| Thickness | the average thickness for at least one of the plates is 2 | For at least one of the plates |
| | nm or less, 10 nm or less, 100 nm or less, 200 nm or | is in the range of 0.5 to 1.5 |
| | less, 500 nm or less, 1000 nm or less, 2 μm (micron) | mm; around 1 mm; in the |
| | or less, 5 μm or less, 10 μm or less, 20 μm or less, 50 | range of 0.15 to 0.2 mm; or |
| | μm or less, 100 μm or less, 150 μm or less, 200 μm or | around 0.175 mm |
| | less, 300 μm or less, 500 μm or less, 800 μm or less, | |
| | 1 mm (millimeter) or less, 2 mm or less, 3 mm or less, | |
| | 5 mm or less, 10 mm or less, 20 mm or less, 50 mm | |
| | or less, 100 mm or less, 500 mm or less, or in a range | |
| | between any two of these values | |
| Lateral | For at least one of the plate is 1 mm2 (square | For at least one plate of the |
| Area | millimeter) or less, 10 mm2 or less, 25 mm2 or less, | QMAX card is in the range of |
| | 50 mm2 or less, 75 mm2 or less, 1 cm2 (square | 500 to 1000 mm²; or around |
| | centimeter) or less, 2 cm2 or less, 3 cm2 or less, 4 | 750 mm². |
| | cm2 or less, 5 cm2 or less, 10 cm2 or less, 100 cm2 or | |
| | less, 500 cm2 or less, 1000 cm2 or less, 5000 cm2 or | |
| | less, 10,000 cm2 or less, 10,000 cm2 or less, or in a | |
| | range between any two of these values | |
| Lateral | For at least one of the plates of the QMAX card is 1 | For at least one plate of the |
| Linear | mm or less, 5 mm or less, 10 mm or less, 15 mm or | QMAX card is in the range |
| Dimension | less, 20 mm or less, 25 mm or less, 30 mm or less, 35 | of 20 to 30 mm; or around 24 |
| (width, | mm or less, 40 mm or less, 45 mm or less, 50 mm or | mm |
| length, or | less, 100 mm or less, 200 mm or less, 500 mm or less, | |
| diameter, | 1000 mm or less, 5000 mm or less, or in a range | |
| etc.) | between any two of these values | |
| Recess | 1 um or less, 10 um or less, 20 um or less, 30 um or | In the range of 1 mm to 10 |
| width | less, 40 um or less, 50 um or less, 100 um or less, 200 | mm; Or |
| | um or less, 300 um or less, 400 um or less, 500 um or | About 5 mm |
| | less, 7500 um or less, 1 mm or less, 5 mm or less, 10 | |
| | mm or less, 100 mm or less, or 1000 mm or less, or in | |
| | a range between any two of these values. | |

### Hinge:

| Parameters | Embodiments | Preferred Embodiments |
|---|---|---|
| Number | 1, 2, 3, 4, 5, or more | 1 or 2 |
| Length of | 1 mm or less, 2 mm or less, 3 mm or less, 4 mm or | In the range of 5 mm to 30 |
| Hinge Joint | less, 5 mm or less, 10 mm or less, 15 mm or less, 20 | mm. |
| | mm or less, 25 mm or less, 30 mm or less, 40 mm or | |
| | less, 50 mm or less, 100 mm or less, 200 mm or | |
| | less, or 500 mm or less, or in a range between any | |
| | two of these values | |
| Ratio (hinge | 1.5 or less, 1 or less, 0.9 or less, 0.8 or less, 0.7 or | In the range of 0.2 to 1; or |
| joint length | less, 0.6 or less, 0.5 or less, 0.4 or less, 0.3 or less, | about 1 |
| vs. aligning | 0.2 or less, 0.1 or less, 0.05 or less or in a range | |
| plate edge | between any two of these values. | |
| length | | |
| Area | 1 mm² or less, 5 mm² or less, 10 mm² or less, 20 mm² | In the range of 20 to 200 |
| | or less, 30 mm² or less, 40 mm² or less, 50 mm² or | mm²; or about 120 mm² |
| | less, 100 mm² or less, 200 mm² or less, 500 mm² or | |
| | less, or in a range between any of the two values | |
| Ratio (hinge | 1 or less, 0.9 or less, 0.8 or less, 0.7 or less, 0.6 or | In the range of 0.05 to 0.2, |
| area vs. | less, 0.5 or less, 0.4 or less, 0.3 or less, 0.2 or less, 0.1 | around 0.15 |
| plate area) | or less, 0.05 or less, 0.01 or less or in a range between | |
| | any two of these values | |
| Max. Open | 15 or less, 30 or less, 45 or less, 60 or less, 75 or less, | In the range of 90 to 180 |
| Degree | 90 or less, 105 or less, 120 or less, 135 or less, 150 or | degrees |
| | less, 165 or less, 180 or less, 195 or less, 210 or less, | |
| | 225 or less, 240 or less, 255 or less, 270 or less, 285 | |
| | or less, 300 or less, 315 or less, 330 or less, 345 or | |
| | less or 360 or less degrees, or in a range between any | |
| | two of these values | |
| No. of | 1, 2, 3, 4, 5, or more | 1 or 2 |
| Layers | | |
| Layer | 0.1 um or less, 1 um or less, 2 um or less, 3 um or less, | In the range of 20 |
| thickness | 5 um or less, 10 um or less, 20 um or less, 30 um or | um to 1 mm; or |
| | less, 50 um or less, 100 um or less, 200 um or less, | Around 50 um |
| | 300 um or less, 500 um or less, 1 mm or less, 2 mm | |
| | or less, and a range between any two of these values | |
| Angle- | Limiting the angle adjustment with no more than | No more than ±2 |
| maintaining | ±90, ±45, ±30, ±25, ±20, ±15, ±10, ±8, ±6, ±5, ±4, | |
| | ±3, ±2, or ±1, or in a range between any two of these | |
| | values | |

### Notch:

| Parameters | Embodiments | Preferred Embodiments |
|---|---|---|
| Number | 1, 2, 3, 4, 5, or more | 1 or 2 |
| Shape | round, ellipse, rectangle, triangle, polygon, ring- | Part of a circle |
| | shaped, or any superposition or portion of these | |
| | shapes. | |
| Positioning | Any location along any edge except the hinge edge, | |
| | or any corner joint by non-hinge edges | |
| Lateral | 1 mm or less, 2.5 mm or less, 5 mm or less, 10 mm | In the range of 5 mm to 15 |
| Linear | or less, 15 mm or less, 20 mm or less, 25 mm or | mm; or about 10 mm |
| Dimension | less, 30 mm or less, 40 mm or less, 50 mm or less, | |
| (Length | or in a range between any two of these values | |
| along the | | |
| edge, radius, | | |
| etc.) | | |
| Area | 1 mm² (square millimeter) or less, 10 mm² or less, 25 | In the range of 10 to 150 |
| | mm² or less, 50 mm² or less, 75 mm² or less or in a | mm²; or about 50 mm² |
| | range between any two of these values. | |

### Trench:

| | | Preferred |
|---|---|---|
| Parameters | Embodiments | Embodiments |
| Number | 1, 2, 3, 4, 5, or more | 1 or 2 |
| Shape | Closed (round, ellipse, rectangle, | |
| | triangle, polygon, ring-shaped, | |
| | or any superposition or portion | |
| | of these shapes) or open-ended | |
| | (straight line, curved line, arc, | |
| | branched tree, or any other shape | |
| | with open endings); | |
| Length | 0.001 mm or less, 0.005 mm or less, | |
| | 0.01 mm or less, 0.05 mm or less, | |
| | 0.1 mm or less, 0.5 mm or less, 1 | |
| | mm or less, 2 mm or less, 5 mm | |
| | or less, 10 mm or less, 20 mm or less, | |
| | 50 mm or less, 100 mm or less, | |
| | or in a range between any two of | |
| | these values | |
| Cross- | 0.001 mm² or less, 0.005 mm² or less, | |
| sectional | 0.01 mm² or less, 0.05 mm² or less, | |
| Area | 0.1 mm² or less, 0.5 mm² or less, | |
| | 1 mm² or less, 2 mm² or less, 5 mm² | |
| | or less, 10 mm² or less, 20 mm² or | |
| | less, or in a range between any | |
| | two of these values. | |
| Volume | 0.1 uL or more, 0.5 uL or more, | In the range of |
| | 1 uL or more, 2 uL or more, 5 uL | 1 uL to 20 |
| | or more, 10 uL or more, 30 uL or | uL; or |
| | more, 50 uL or more, 100 uL or | About 5 uL |
| | more, 500 uL or more, 1 mL or | |
| | more, or in a range between any two | |
| | of these values | |

### Receptacle Slot

| | | Preferred |
|---|---|---|
| Parameters | Embodiments | Embodiments |
| Shape of | round, ellipse, rectangle, triangle, | |
| receiving | polygon, ring-shaped, or any | |
| area | superposition of these shapes; | |
| Difference | 100 nm, 500 nm, 1 um, 2 um, 5 um, | In the range of |
| between | 10 um, 50 um, 100 um, 300 um, | 50 to 300 um; |
| sliding track | 500 um, 1 mm, 2 mm 5 mm, 1 cm, | or about 75 um |
| gap size and | or in a range between any two of | |
| card | the values. | |
| thickness | | |

### (2) Applications

The devices/apparatus, systems, and methods herein disclosed can be used for various applications (fields and samples). The applications are herein disclosed, listed, described, and/or summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

In some embodiments, the devices, apparatus, systems, and methods herein disclosed are used in a variety of different application in various field, wherein determination of the presence or absence, quantification, and/or amplification of one or more analytes in a sample are desired. For example, in certain embodiments the subject devices, apparatus, systems, and methods are used in the detection of proteins, peptides, nucleic acids, synthetic compounds, inorganic compounds, organic compounds, bacteria, virus, cells, tissues, nanoparticles, and other molecules, compounds, mixtures and substances thereof. The various fields in which the subject devices, apparatus, systems, and methods can be used include, but are not limited to: diagnostics, management, and/or prevention of human diseases and conditions, diagnostics, management, and/or prevention of veterinary diseases and conditions, diagnostics, management, and/or prevention of plant diseases and conditions, agricultural uses, veterinary uses, food testing, environments testing and decontamination, drug testing and prevention, and others.

The applications of the present invention include, but are not limited to: (a) the detection, purification, quantification, and/or amplification of chemical compounds or biomolecules that correlates with certain diseases, or certain stages of the diseases, e.g., infectious and parasitic disease, injuries, cardiovascular disease, cancer, mental disorders, neuropsychiatric disorders and organic diseases, e.g., pulmonary diseases, renal diseases, (b) the detection, purification, quantification, and/or amplification of cells and/or microorganism, e.g., virus, fungus and bacteria from the environment, e.g., water, soil, or biological samples, e.g., tissues, bodily fluids, (c) the detection, quantification of chemical compounds or biological samples that pose hazard to food safety, human health, or national security, e.g. toxic waste, anthrax, (d) the detection and quantification of vital parameters in medical or physiological monitor, e.g., glucose, blood oxygen level, total blood count, (e) the detection and quantification of specific DNA or RNA from biological samples, e.g., cells, viruses, bodily fluids, (f) the sequencing and comparing of genetic sequences in DNA in the chromosomes and mitochondria for genome analysis or (g) the detection and quantification of reaction products, e.g., during synthesis or purification of pharmaceuticals.

In some embodiments, the subject devices, apparatus, systems, and methods are used in the detection of nucleic acids, proteins, or other molecules or compounds in a sample. In certain embodiments, the devices, apparatus, systems, and methods are used in the rapid, clinical detection and/or quantification of one or more, two or more, or three or more disease biomarkers in a biological sample, e.g., as being employed in the diagnosis, prevention, and/or management of a disease condition in a subject. In certain embodiments, the devices, apparatus, systems, and methods are used in the detection and/or quantification of one or more, two or more, or three or more environmental markers in an environmental sample, e.g. sample obtained from a river, ocean, lake, rain, snow, sewage, sewage processing runoff, agricultural runoff, industrial runoff, tap water or drinking water. In certain embodiments, the devices, apparatus, systems, and methods are used in the detection and/or quantification of one or more, two or more, or three or more foodstuff marks from a food sample obtained from tap water, drinking water, prepared food, processed food or raw food.

In some embodiments, the subject device is part of a microfluidic device. In some embodiments, the subject devices, apparatus, systems, and methods are used to detect a fluorescence or luminescence signal. In some embodiments, the subject devices, apparatus, systems, and methods include, or are used together with, a communication device, such as but not limited to: mobile phones, tablet computers and laptop computers. In some embodiments, the subject devices, apparatus, systems, and methods include, or are used together with, an identifier, such as but not limited to an optical barcode, a radio frequency ID tag, or combinations thereof.

In some embodiments, the sample is a diagnostic sample obtained from a subject, the analyte is a biomarker, and the measured amount of the analyte in the sample is diagnostic of a disease or a condition. In some embodiments, the subject devices, systems and methods further include receiving or providing to the subject a report that indicates the measured amount of the biomarker and a range of measured values for the biomarker in an individual free of or at low risk of having the disease or condition, wherein the measured amount of the biomarker relative to the range of measured values is diagnostic of a disease or condition.

In some embodiments, the sample is an environmental sample, and wherein the analyte is an environmental marker. In some embodiments, the subject devices, systems and methods includes receiving or providing a report that indicates the safety or harmfulness for a subject to be exposed to the environment from which the sample was obtained. In some embodiments, the subject devices, systems and methods include sending data containing the measured amount of the environmental marker to a remote location and receiving a report that indicates the safety or harmfulness for a subject to be exposed to the environment from which the sample was obtained.

In some embodiments, the sample is a foodstuff sample, wherein the analyte is a foodstuff marker, and wherein the amount of the foodstuff marker in the sample correlate with safety of the foodstuff for consumption. In some embodiments, the subject devices, systems and methods include receiving or providing a report that indicates the safety or harmfulness for a subject to consume the foodstuff from which the sample is obtained. In some embodiments, the subject devices, systems and methods include sending data containing the measured amount of the foodstuff marker to a remote location and receiving a report that indicates the safety or harmfulness for a subject to consume the foodstuff from which the sample is obtained.

### 9.2 Hinges, Opening Notches, Recessed Edge and Sliders

The devices, systems, and methods herein disclosed can include or use Q-cards for sample detection, analysis, and quantification. In some embodiments, the Q-card comprises hinges, notches, recesses, and sliders, which help to facilitate the manipulation of the Q card and the measurement of the samples. The structure, material, function, variation and dimension of the hinges, notches, recesses, and sliders are herein disclosed, or listed, described, and summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, all of which applications are incorporated herein in their entireties for all purposes.

### 9.3 Q-Card, Sliders, and Smartphone Detection System

The devices, systems, and methods herein disclosed can include or use Q-cards for sample detection, analysis, and quantification. In some embodiments, the Q-cards are used together with sliders that allow the card to be read by a smartphone detection system. The structure, material, function, variation, dimension and Connection of the Q-card, the sliders, and the smartphone detection system are herein disclosed, or listed, described, and summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, all of which applications are incorporated herein in their entireties for all purposes.

### 9.4 Detection Methods

The devices, systems, and methods herein disclosed can include or be used in various types of detection methods. The detection methods are herein disclosed, or listed, described, and summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, all of which applications are incorporated herein in their entireties for all purposes.

### 9.5 Labels

The devices, systems, and methods herein disclosed can employ various types of labels that are used for analytes detection. The labels are herein disclosed, or listed, described, and summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, all of which applications are incorporated herein in their entireties for all purposes.

### 9.6 Analytes

The devices, systems, and methods herein disclosed can be applied to manipulation and detection of various types of analytes (including biomarkers). The analytes and are herein disclosed, or listed, described, and summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, all of which applications are incorporated herein in their entireties for all purposes.

### 9.7 Applications (Field and Samples)

The devices, systems, and methods herein disclosed can be used for various applications (fields and samples). The applications are herein disclosed, or listed, described, and summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, all of which applications are incorporated herein in their entireties for all purposes.

### 9.8 Cloud

The devices, systems, and methods herein disclosed can employ cloud technology for data transfer, storage, and/or analysis. The related cloud technologies are herein disclosed, or listed, described, and summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, all of which applications are incorporated herein in their entireties for all purposes.

It must be noted that as used herein and in the appended claims, the singular forms “a”, “an”, and “the” include plural referents unless the context clearly dictates otherwise, e.g., when the word “single” is used. For example, reference to “an analyte” includes a single analyte and multiple analytes, reference to “a capture agent” includes a single capture agent and multiple capture agents, reference to “a detection agent” includes a single detection agent and multiple detection agents, and reference to “an agent” includes a single agent and multiple agents.

As used herein, the terms “adapted” and “configured” mean that the element, component, or other subject matter is designed and/or intended to perform a given function. Thus, the use of the terms “adapted” and “configured” should not be construed to mean that a given element, component, or other subject matter is simply “capable of” performing a given function. Similarly, subject matter that is recited as being configured to perform a particular function may additionally or alternatively be described as being operative to perform that function.

As used herein, the phrase, “for example,” the phrase, “as an example,” and/or simply the terms “example” and “exemplary” when used with reference to one or more components, features, details, structures, embodiments, and/or methods according to the present disclosure, are intended to convey that the described component, feature, detail, structure, embodiment, and/or method is an illustrative, non-exclusive example of components, features, details, structures, embodiments, and/or methods according to the present disclosure. Thus, the described component, feature, detail, structure, embodiment, and/or method is not intended to be limiting, required, or exclusive/exhaustive; and other components, features, details, structures, embodiments, and/or methods, including structurally and/or functionally similar and/or equivalent components, features, details, structures, embodiments, and/or methods, are also within the scope of the present disclosure.

As used herein, the phrases “at least one of” and “one or more of,” in reference to a list of more than one entity, means any one or more of the entity in the list of entity, and is not limited to at least one of each and every entity specifically listed within the list of entity. For example, “at least one of A and B” (or, equivalently, “at least one of A or B,” or, equivalently, “at least one of A and/or B”) may refer to A alone, B alone, or the combination of A and B.

As used herein, the term “and/or” placed between a first entity and a second entity means one of (1) the first entity, (2) the second entity, and (3) the first entity and the second entity.

Multiple entity listed with “and/or” should be construed in the same manner, i.e., “one or more” of the entity so conjoined. Other entity may optionally be present other than the entity specifically identified by the “and/or” clause, whether related or unrelated to those entities specifically identified.

Where numerical ranges are mentioned herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, unless otherwise indicated or otherwise evident from the context and understanding of one of ordinary skill in the art.

In the event that any patents, patent applications, or other references are incorporated by reference herein and (1) define a term in a manner that is inconsistent with and/or (2) are otherwise inconsistent with, either the non-incorporated portion of the present disclosure or any of the other incorporated references, the non-incorporated portion of the present disclosure shall control, and the term or incorporated disclosure therein shall only control with respect to the reference in which the term is defined and/or the incorporated disclosure was present originally.

It is believed that the following claims particularly point out certain combinations and subcombinations that are directed to one of the disclosed inventions and are novel and nonobvious. Inventions embodied in other combinations and subcombinations of features, functions, elements and/or properties may be claimed through amendment of the present claims or presentation of new claims in this or a related application. Such amended or new claims, whether they are directed to a different invention or directed to the same invention, whether different, broader, narrower, or equal in scope to the original claims, are also regarded as included within the subject matter of the inventions of the present disclosure.

1. A device for assaying a sample, comprising:
a first plate, a second plate, spacers, and a sponge, wherein:
i. the plates are movable relative to each other into different configurations,
ii. the first plate comprises, on its inner surface, a sample contact area for contacting a sample that contains or is suspected to contains an analyte,
iii. the spacers are fixed on respective surfaces of one or both of the plates, the spacers having a predetermined substantially uniform height and a predetermined fixed inter-spacer distance, and
iv. the sponge is made of a flexible porous material capable of absorbing or releasing a liquid;
wherein the spacers reduce direct contact between the sponge and the surface of the plate when the sponge is pressed against the plate surface that has spacers;
wherein one of the configurations is an open configuration, in which:
the two plates are partially or completely separated apart,
the spacing between the plates is not regulated by the spacers, allowing the sample to be deposited on one or both of the plates,
wherein another of the configurations is a closed configuration which is configured after the sample is deposited in the open configuration; and in the closed configuration:
at least part of the sample is compressed by the two plates into a layer of highly uniform thickness, and the uniform thickness of the layer is confined by the inner surfaces of the two plates and is regulated by the plates and the spacers, and
wherein a washing configuration is configured when the second plate is separated from the first plate after the closed configuration; and in the washing configuration:
the sponge containing a wash solution is placed on the sample contact area of the first plate, and
the sponge, when pressed, fills the sample contact area with the wash solution, and, when the press is relieved, re-absorbs the wash solution.

2. A method of assaying a sample, comprising:
(a) obtaining a first plate, a second plate, and spacers, wherein:
i. the plates are movable relative to each other into different configurations;
ii. the first plate comprises, on its inner surface, a sample contact area for contacting a sample that comprises an analyte,
iii. one or both of the plates comprise the spacers that are fixed on the inner surface of a respective plate; and
iv. the spacers have a predetermined substantially uniform height and a predetermined inter-spacer-distance;
(b) depositing a liquid sample on a sample contact area of the first plate in an open configuration, in which the two plates are partly or entirely separated apart;
(c) pressing the plates into a closed configuration, in which at least part of the sample is compressed into a layer of uniform thickness by the first and second plates and incubating the sample for a predetermined period of time,
(d) removing the second plate,
(e) placing a sponge containing a wash solution on the spacers in the sample contact area of the first plate, wherein the spacers prevent contact between the sponge and the surface of the first plate,
(f) pressing the sponge to deposit the wash solution onto the sample contact area, holding the sponge at the pressed position for a period of time, and releasing the sponge to reabsorb the wash solution.

3. The device of claim 1, wherein the sample comprises a bodily fluid selected from the group consisting of: amniotic fluid, aqueous humour, vitreous humour, blood (e.g., whole blood, fractionated blood, plasma or serum), breast milk, cerebrospinal fluid (CSF), cerumen (earwax), chyle, chime, endolymph, perilymph, feces, breath, gastric acid, gastric juice, lymph, mucus (including nasal drainage and phlegm), pericardial fluid, peritoneal fluid, pleural fluid, pus, rheum, saliva, exhaled breath condensate, sebum, semen, sputum, sweat, synovial fluid, tears, vomit, urine, and a combination thereof.

4. The device of claim 1, wherein the sample is blood.

5. The device of claim 1, wherein the sample is an environmental sample from an environmental source selected from the group consisting of a river, lake, pond, ocean, glaciers, icebergs, rain, snow, sewage, reservoirs, tap water, drinking water, soil, compost, sand, rocks, concrete, wood, brick, sewage, the air, underwater heat vents, industrial exhaust, vehicular exhaust, and a combination thereof.

6. The device of claim 1, wherein the sample is a foodstuff sample selected from the group consisting of: raw ingredients, cooked food, plant and animal sources of food, preprocessed food, partially or fully processed food, and a combination thereof.

7. The device of claim 1, wherein the spacers have a filling factor of at least 1%, the filling factor being the ratio of the spacer area in the sample contact surface to the total area of the sample contact surface.

8. The device of claim 1, wherein the Young's modulus of the spacers times the filling factor of the spacers is equal or larger than 10 MPa, the filling factor being the ratio of the spacer area in the sample contact surface to the total area of the sample contact surface.

9. The device of claim 1, wherein the inter-spacer distance is in the range of 1 μm to 200 μm and the inter-spacer distance is substantially periodic.

10. The device of claim 1, wherein the inter-spacer distance is in the range of 7 μm to 200 μm and the sample is blood.

11. The device of claim 1, wherein the spacers have a density of at least 100/mm².

12. The device of claim 1, wherein the spacers have a density of at least 1000/mm².

13. The device of claim 1, wherein the spacers are pillars with a cross-sectional shape selected from round, polygonal, circular, square, rectangular, oval, elliptical, or any combination of the same.

14. The device of claim 1, wherein the average thickness of the layer of uniform thickness has a value equal to or less than 1 μm.

15. The device of claim 1, wherein the average thickness of the layer of uniform thickness has a value in the range of 1 μm to 10 μm.

16. The device of claim 1, wherein the average thickness of the layer of uniform thickness has a value in the range of 10 μm to 30 μm.

17. The device of claim 1, wherein the average thickness of the layer of uniform thickness has a value in the range of 2 μm to 3.8 μm and the sample is blood.

18. The device of claim 1, wherein the average thickness of the layer of uniform thickness has a value in the range of 1 μm to 10 μm and the sample is exhaled breath condensate.

19. The device of claim 1, wherein the materials of the plate and the spacers are selected from polystyrene, PMMA, PC, COC, COP, or another plastic.

20. The device of claim 1, wherein the first and second plates are connected and are configured to be changed from the open configuration to the closed configuration by folding the plates.

21. The device of claim 1, wherein the first and second plates are connected by a hinge and are configured to be changed from the open configuration to the closed configuration by folding the plates along the hinge.

22. The device of claim 1, wherein the first and second plates are connected by a hinge that is a separate material to the plates, and are configured to be changed from the open configuration to the closed configuration by folding the plates along the hinge.

23. The device of claim 1, wherein the sponge comprises a porous substrate and said porous substrate contains pores of a diameter in the range of 10 nm to 100 nm, 100 nm to 500 nm, 500 nm to 1 μm, 1 μm to 10 μm, 10 μm to 50 μm, 50 μm to 100 μm, 100 μm to 500 μm, 500 μm to 1 mm.

24. The device of claim 1, wherein the sponge comprises a porous substrate and said porous substrate contains pores of a diameter in the range of 500 nm to 1 μm, 1 μm to 10 μm, 10 μm to 50 μm, 50 μm to 100 μm, 100 μm to 500 μm.

25. The device of claim 1, wherein the sponge comprises a porous substrate and said porous substrate possesses a porosity in the range of 10 to 20%, 20 to 30%, 30 to 40%, 40 to 50%, 50 to 60%, 60 to 70%, 70 to 80%, 80 to 90%, 90 to 99%.

26. The device of claim 1, wherein said the sponge comprises a porous substrate and said porous substrate possesses a porosity in the range of 70 to 80%, 80 to 90%, 90 to 99%.

27. The device of claim 1, wherein the sponge comprises a porous substrate and the materials of said porous substrate contains rubber, cellulose, cellulose wood fibers, foamed plastic polymers, low-density polyether, polyvinyl alcohol (pva), polyester, poly(methyl methacrylate) (PMMA), polystyrene, etc.

28. The method of claim 2, further comprising: after the step (f), detecting the analyte bound to the capture agents.

29. The method of claim 2, wherein the detecting includes measuring at least one of fluorescence, luminescence, scattering, reflection, absorbance, and surface plasmon resonance associated with the analyte bound to the capture agents.

30. The method of claim 2, wherein the inner surface of the first plate at the assay site includes a signal amplification surface such as a metal and/or dielectric microstructure.

31. The method of claim 2, wherein the sample comprises a bodily fluid selected from the group consisting of: amniotic fluid, aqueous humour, vitreous humour, blood (e.g., whole blood, fractionated blood, plasma or serum), breast milk, cerebrospinal fluid (CSF), cerumen (earwax), chyle, chime, endolymph, perilymph, feces, breath, gastric acid, gastric juice, lymph, mucus (including nasal drainage and phlegm), pericardial fluid, peritoneal fluid, pleural fluid, pus, rheum, saliva, exhaled breath condensate, sebum, semen, sputum, sweat, synovial fluid, tears, vomit, urine, and a combination thereof.

32. The method of claim 2, wherein the sample is blood.

33. The method of claim 2, wherein the sample is an environmental sample from an environmental source selected from the group consisting of a river, lake, pond, ocean, glaciers, icebergs, rain, snow, sewage, reservoirs, tap water, drinking water, soil, compost, sand, rocks, concrete, wood, brick, sewage, the air, underwater heat vents, industrial exhaust, vehicular exhaust, and a combination thereof.

34. The method of claim 2, wherein the sample is a foodstuff sample selected from the group consisting of: raw ingredients, cooked food, plant and animal sources of food, preprocessed food, partially or fully processed food, and a combination thereof.

35. The method of claim 2, wherein the spacers have a filling factor of at least 1%, the filling factor being the ratio of the spacer area in the sample contact surface to the total area of the sample contact surface.

36. The method of claim 2, wherein the Young's modulus of the spacers times the filling factor of the spacers is equal or larger than 10 MPa, the filling factor being the ratio of the spacer area in the sample contact surface to the total area of the sample contact surface.

37. The method of claim 2, wherein the inter-spacer distance is in the range of 1 μm to 200 μm and the inter-spacer distance is substantially periodic.

38. The method of claim 2, wherein the inter-spacer distance is in the range of 7 μm to 200 μm and the sample is blood.

39. The method of claim 2, wherein the spacers have a density of at least 100/mm².

40. The method of claim 2, wherein the spacers have a density of at least 1000/mm².

41. The method of claim 2, wherein the spacers are pillars with a cross-sectional shape selected from round, polygonal, circular, square, rectangular, oval, elliptical, or any combination of the same.

42. The method of claim 2, wherein the average thickness of the layer of uniform thickness has a value equal to or less than 1 μm.

43. The method of claim 2, wherein the average thickness of the layer of uniform thickness has a value in the range of 1 μm to 10 μm.

44. The method of claim 2, wherein the average thickness of the layer of uniform thickness has a value in the range of 10 μm to 30 μm.

45. The method of claim 2, wherein the average thickness of the layer of uniform thickness has a value in the range of 2 μm to 3.8 μm and the sample is blood.

46. The method of claim 2, wherein the average thickness of the layer of uniform thickness has a value in the range of 1 μm to 10 μm and the sample is exhaled breath condensate.

47. The method of claim 2, wherein the materials of the plate and the spacers are selected from polystyrene, PMMA, PC, COC, COP, or another plastic.

48. The method of claim 2, wherein the first and second plates are connected and are configured to be changed from the open configuration to the closed configuration by folding the plates.

49. The method of claim 2, wherein the first and second plates are connected by a hinge and are configured to be changed from the open configuration to the closed configuration by folding the plates along the hinge.

50. The method of claim 2, wherein the first and second plates are connected by a hinge that is a separate material to the plates, and are configured to be changed from the open configuration to the closed configuration by folding the plates along the hinge.

51. The method of claim 2, wherein the sponge comprises a porous substrate and said porous substrate contains pores of a diameter in the range of 10 nm to 100 nm, 100 nm to 500 nm, 500 nm to 1 μm, 1 μm to 10 μm, 10 μm to 50 μm, 50 μm to 100 μm, 100 μm to 500 μm, 500 μm to 1 mm.

52. The method of claim 2, wherein the sponge comprises a porous substrate and said porous substrate contains pores of a diameter in the range of 500 nm to 1 μm, 1 μm to 10 μm, 10 μm to 50 μm, 50 μm to 100 μm, 100 μm to 500 μm.

53. The method of claim 2, wherein the sponge comprises a porous substrate and said porous substrate possesses a porosity in the range of 10 to 20%, 20 to 30%, 30 to 40%, 40 to 50%, 50 to 60%, 60 to 70%, 70 to 80%, 80 to 90%, 90 to 99%.

54. The method of claim 2, wherein said the sponge comprises a porous substrate and said porous substrate possesses a porosity in the range of 70 to 80%, 80 to 90%, 90 to 99%.

55. The method of claim 2, wherein the sponge comprises a porous substrate and the materials of said porous substrate contains rubber, cellulose, cellulose wood fibers, foamed plastic polymers, low-density polyether, polyvinyl alcohol (pva), polyester, poly(methyl methacrylate) (PMMA), polystyrene, etc.

56. A method for determining a dilution factor for a diluted sample, comprising the steps of:
(a) providing an initial sample containing a calibration marker;
(b) obtaining a first concentration of the calibration marker in the initial sample;
(c) diluting the initial sample with an unknown volume of a diluent to form a diluted sample;
(d) obtaining, after (c), a second concentration of the calibration marker using a concentration-measuring device; and
(e) determining the dilution factor by comparing the first concentration and the second concentration,
wherein the concentration-measuring device comprises:
a first plate, a second plate, spacers, and a detector, wherein:
i. the plates are movable relative to each other into different configurations;
ii. one or both plates are flexible;
iii. each of the plates has, on its respective surface, a sample contact area for contacting a sample that contains an analyte,
iv. one or both of the plates comprise spacers that are fixed on the inner surface of a respective plate,
v. the spacers have a predetermined substantially uniform height and a predetermined constant inter-spacer distance and at least one of the spacers is inside the sample contact area, and
vi. a detector that detects the analyte;
wherein one of the configurations is an open configuration, in which:
the two plates are partially or entirely separated apart,
the spacing between the plates is not regulated by the spacers, and the sample is deposited on one or both of the plates; and
wherein another of the configurations is a closed configuration which is configured after the sample is deposited in the open configuration; and in the closed configuration:
at least part of the sample is compressed by the two plates into a layer of uniform thickness,
the layer of uniform thickness, confined by the inner surfaces of the two plates, is regulated by the plates and the spacers, and has an average thickness equal to or less than 5 μm with a small variation, and the detector detects the analyte and calculates a concentration of the analyte in the sample.

57. The method of claim 56, wherein in the step of (b), the first concentration of the calibration marker, if unknown, is obtained using the concentration-measuring device.

58. The method of claim 56, wherein the sample comprises a bodily fluid selected from the group consisting of: amniotic fluid, aqueous humour, vitreous humour, blood (e.g., whole blood, fractionated blood, plasma or serum), breast milk, cerebrospinal fluid (CSF), cerumen (earwax), chyle, chime, endolymph, perilymph, feces, breath, gastric acid, gastric juice, lymph, mucus (including nasal drainage and phlegm), pericardial fluid, peritoneal fluid, pleural fluid, pus, rheum, saliva, exhaled breath condensate, sebum, semen, sputum, sweat, synovial fluid, tears, vomit, urine, and a combination thereof.

59. The method of claim 56, wherein the sample is blood.

60. The method of claim 56, wherein the sample is an environmental sample from an environmental source selected from the group consisting of a river, lake, pond, ocean, glaciers, icebergs, rain, snow, sewage, reservoirs, tap water, drinking water, soil, compost, sand, rocks, concrete, wood, brick, sewage, the air, underwater heat vents, industrial exhaust, vehicular exhaust, and a combination thereof.

61. The method of claim 56, wherein the sample is a foodstuff sample selected from the group consisting of: raw ingredients, cooked food, plant and animal sources of food, preprocessed food, partially or fully processed food, and a combination thereof.

62. The method of claim 56, wherein one or both plates comprises a location marker, either on a surface of or inside the plate, that provide information of a location of the plate.

63. The method of claim 56, wherein one or both plates comprises a Scale marker, either on a surface of or inside the plate, that provide information of a lateral dimension of a structure of the sample and/or the plate.

64. The method of claim 56, wherein one or both plates comprises an imaging marker, either on surface of or inside the plate, that assists an imaging of the sample.

65. The method of claim 56, wherein the spacers functions as a location marker, a scale marker, an imaging marker, or any combination of thereof.

66. The method of claim 56, wherein the average thickness of the layer of uniform thickness is in the range of 2 μm to 2.2 μm and the sample is blood.

67. The method of claim 56, wherein the average thickness of the layer of uniform thickness is in the range of 2.2 μm to 2.6 μm and the sample is blood.

68. The method of claim 56, wherein the average thickness of the layer of uniform thickness is in the range of 1.8 μm to 2 μm and the sample is blood.

69. The method of claim 56, wherein the average thickness of the layer of uniform thickness is in the range of 2.6 μm to 3.8 μm and the sample is blood.

70. The method of claim 56, wherein the average thickness of the layer of uniform thickness is in the range of 1.8 μm to 3.8 μm and the sample is whole blood without a dilution by another liquid.

71. The method of claim 56, wherein the spacers have a filling factor of at least 1%, the filling factor being the ratio of the spacer area in the sample contact surface to the total area of the sample contact surface.

72. The method of claim 56, wherein the Young's modulus of the spacers times the filling factor of the spacers is equal or larger than 10 MPa, the filling factor being the ratio of the spacer area in the sample contact surface to the total area of the sample contact surface.

73. The method of claim 56, wherein the inter-spacer distance is in the range of 1 μm to 200 μm and the inter-spacer distance is substantially periodic.

74. The method of claim 56, wherein the inter-spacer distance is in the range of 7 μm to 200 μm and the sample is blood.

75. The method of claim 56, wherein the spacers have a density of at least 100/mm².

76. The method of claim 56, wherein the spacers have a density of at least 1000/mm².

77. The method of claim 56, wherein the spacers are pillars with a cross-sectional shape selected from round, polygonal, circular, square, rectangular, oval, elliptical, or any combination of the same.

78. The device of claim 56, wherein the materials of the plate and the spacers are selected from polystyrene, PMMA, PC, COC, COP, or another plastic.

79. A device for sample analysis, comprising:
a first plate, a second plate, spacers, and a filter, wherein:
i. the plates are movable relative to each other into different configurations;
ii. the spacers are fixed on the inner surface of one or more of the plates, the spacers having a predetermined substantially uniform height and a predetermined inter-spacer-distance;
iii. the filter, having a sample receiving surface and a sample exit surface, is placed on top of the first plate with the sample exit surface facing the inner surface of the first plate; and
iv. the sample receiving surface of the filter is to deposit a liquid sample comprising one or more components;
wherein one of the configurations is an depositing configuration, in which:
the second plate is separated, partially or completely, from the first plate and the filter;
the sample is deposited on the sample receiving surface of the filter; and
the distance between the first plate and the second plate is not regulated by their spacers, the filter, or the deposited sample; and
wherein another of the configurations is a filtering configuration, in which:
the filter is positioned between the first plate and the second plate,
the distance between the first plate and the second plate is regulated by their spacers, the filter, and the deposited sample, and
the inner surface of the second plate presses the deposited sample against the filter, forcing at least one component of the sample to flow through the filter toward the first plate, thereby separating the at least one component from the sample.

80. A method for sample analysis, comprising the steps of:
(a) obtaining a liquid sample;
(b) obtaining a first plate, a second plate, spacers, and a filter, wherein:
i. the plates are movable relative to each other into different configurations;
ii. one or both of the plates comprise the spacers that are fixed on the inner surface of a respective plate;
iii. the spacers have a predetermined substantially uniform height and a predetermined inter-spacer-distance;
iv. the filter, having a sample receiving surface and a sample exit surface, is placed on top of the first plate with the sample exit surface facing the inner surface of the first plate;
(c) depositing the sample on a sample receiving surface of the filter when the plates are in a depositing configuration, in which:
the two plates are partially or entirely separated apart, and
the spacing between the plates is not regulated by the spacers, the filter, or the deposited sample; and
(d) after (c), bringing the two plates together; and
conformable pressing, either in parallel or sequentially, an area of at least one of the plates to press the plates together to a filtering configuration, wherein:
the inner surface of the second plate presses the deposited sample against the filter, forcing at least one component of the sample to flow through the filter toward the first plate, thereby separating the at least one component from the sample,
the conformable pressing generates a substantially uniform pressure on the plates,
the conformable pressing makes the pressure applied over an area is substantially constant regardless the shape variation of the outer surfaces of the plates,
the conformable pressing in parallel applies the pressures on the intended area at the same time,
the conformable pressing sequentially applies the pressure on a part of the intended area and gradually move to other area, and
in the filtering configuration, the spacing between the plates in the layer of uniform thickness region is regulated by the spacers, the filter, and the deposited sample.

81. The device of claim 79, wherein the sample comprises a bodily fluid selected from the group consisting of: amniotic fluid, aqueous humour, vitreous humour, blood (e.g., whole blood, fractionated blood, plasma or serum), breast milk, cerebrospinal fluid (CSF), cerumen (earwax), chyle, chime, endolymph, perilymph, feces, breath, gastric acid, gastric juice, lymph, mucus (including nasal drainage and phlegm), pericardial fluid, peritoneal fluid, pleural fluid, pus, rheum, saliva, exhaled breath condensate, sebum, semen, sputum, sweat, synovial fluid, tears, vomit, urine, and a combination thereof.

82. The device of claim 79, wherein the sample is blood.

83. The device of claim 79, wherein the sample is an environmental sample from an environmental source selected from the group consisting of a river, lake, pond, ocean, glaciers, icebergs, rain, snow, sewage, reservoirs, tap water, drinking water, soil, compost, sand, rocks, concrete, wood, brick, sewage, the air, underwater heat vents, industrial exhaust, vehicular exhaust, and a combination thereof.

84. The device of claim 79, wherein the sample is a foodstuff sample selected from the group consisting of: raw ingredients, cooked food, plant and animal sources of food, preprocessed food, partially or fully processed food, and a combination thereof.

85. The device of claim 79, wherein the spacers have a filling factor of at least 1%, the filling factor being the ratio of the spacer area in the sample contact surface to the total area of the sample contact surface.

86. The device of claim 79, wherein the Young's modulus of the spacers times the filling factor of the spacers is equal or larger than 10 MPa, the filling factor being the ratio of the spacer area in the sample contact surface to the total area of the sample contact surface.

87. The device of claim 79, wherein the inter-spacer distance is in the range of 1 μm to 200 μm and the inter-spacer distance is substantially periodic.

88. The device of claim 79, wherein the inter-spacer distance is in the range of 7 μm to 200 μm and the sample is blood.

89. The device of claim 79, wherein the spacers have a density of at least 100/mm².

90. The device of claim 79, wherein the spacers have a density of at least 1000/mm².

91. The device of claim 79, wherein the spacers are pillars with a cross-sectional shape selected from round, polygonal, circular, square, rectangular, oval, elliptical, or any combination of the same.

92. The device of claim 79, wherein the average thickness of the layer of uniform thickness has a value equal to or less than 1 μm.

93. The device of claim 79, wherein the average thickness of the layer of uniform thickness has a value in the range of 1 μm to 10 μm.

94. The device of claim 79, wherein the average thickness of the layer of uniform thickness has a value in the range of 10 μm to 30 μm.

95. The device of claim 79, wherein the average thickness of the layer of uniform thickness has a value in the range of 2 μm to 3.8 μm and the sample is blood.

96. The device of claim 79, wherein the materials of the plate and the spacers are selected from polystyrene, PMMA, PC, COC, COP, or another plastic.

97. The method of claim 80, wherein the sample comprises a bodily fluid selected from the group consisting of: amniotic fluid, aqueous humour, vitreous humour, blood (e.g., whole blood, fractionated blood, plasma or serum), breast milk, cerebrospinal fluid (CSF), cerumen (earwax), chyle, chime, endolymph, perilymph, feces, breath, gastric acid, gastric juice, lymph, mucus (including nasal drainage and phlegm), pericardial fluid, peritoneal fluid, pleural fluid, pus, rheum, saliva, exhaled breath condensate, sebum, semen, sputum, sweat, synovial fluid, tears, vomit, urine, and a combination thereof.

98. The method of claim 80, wherein the sample is blood.

99. The method of claim 80, wherein the sample is an environmental sample from an environmental source selected from the group consisting of a river, lake, pond, ocean, glaciers, icebergs, rain, snow, sewage, reservoirs, tap water, drinking water, soil, compost, sand, rocks, concrete, wood, brick, sewage, the air, underwater heat vents, industrial exhaust, vehicular exhaust, and a combination thereof.

100. The method of claim 80, wherein the sample is a foodstuff sample selected from the group consisting of: raw ingredients, cooked food, plant and animal sources of food, preprocessed food, partially or fully processed food, and a combination thereof.

101. The method of claim 80, wherein the spacers have a filling factor of at least 1%, the filling factor being the ratio of the spacer area in the sample contact surface to the total area of the sample contact surface.

102. The method of claim 80, wherein the Young's modulus of the spacers times the filling factor of the spacers is equal or larger than 10 MPa, the filling factor being the ratio of the spacer area in the sample contact surface to the total area of the sample contact surface.

103. The method of claim 80, wherein the inter-spacer distance is in the range of 1 μm to 200 μm and the inter-spacer distance is substantially periodic.

104. The method of claim 80, wherein the inter-spacer distance is in the range of 7 μm to 200 μm and the sample is blood.

105. The method of claim 80, wherein the spacers have a density of at least 100/mm².

106. The method of claim 80, wherein the spacers have a density of at least 1000/mm².

107. The method of claim 80, wherein the spacers are pillars with a cross-sectional shape selected from round, polygonal, circular, square, rectangular, oval, elliptical, or any combination of the same.

108. The method of claim 80, wherein the average thickness of the layer of uniform thickness has a value equal to or less than 1 μm.

109. The method of claim 80, wherein the average thickness of the layer of uniform thickness has a value in the range of 1 μm to 10 μm.

110. The method of claim 80, wherein the average thickness of the layer of uniform thickness has a value in the range of 10 μm to 30 μm.

111. The method of claim 80, wherein the average thickness of the layer of uniform thickness has a value in the range of 2 μm to 3.8 μm and the sample is blood.

112. The method of claim 80, wherein the materials of the plate and the spacers are selected from polystyrene, PMMA, PC, COC, COP, or another plastic.

113. A device for sample analysis, comprising:
a first plate, a second plate, a third plate, and spacers, wherein:
i. the second plate and the third plate are respectively connected to the first plate, wherein the second plate and the third plate are configured to each pivot against the first plate without interfering with each other,
ii. by pivoting against the first plate, either the second plate or the third plate is movable relative to the first plate into different configurations,
iii. the first plate comprises an inner surface that has a sample contact area for contacting a liquid sample, and
iv. the spacers are fixed on the inner surface of one or more of the plates or are mixed in the sample, the spacers having a predetermined substantially uniform height and a predetermined inter-spacer-distance; and
wherein one of the configurations is an open configuration, in which:
all three plates are partially or entirely separated apart,
the spacing between the plates is not regulated by the spacers, and
the sample is deposited on the inner surface of the first plate, the second plate, or both; and
wherein another of the configurations is a closed configuration which is configured after the sample is deposited in the open configuration, and in the closed configuration:
at least part of the sample deposited is compressed by the first plate and the second plate into a layer of uniform thickness, and
the uniform thickness of the layer is confined by the inner surfaces of the first and second plates and is regulated by the plates and the spacers.

114. The device of claim 113, further comprising a filter, wherein:
the filter, having a sample receiving surface and a sample exit surface, is placed on top of the first plate with the sample exit surface facing the inner surface of the first plate;
in the open configuration:
all three plates are partially or entirely separated apart,
the spacing between the plates is not regulated by the spacers, and
a sample comprising one or more components is deposited on the sample receiving surface of the filter;
a filtering configuration is configured after the sample is deposited in the open configuration, and in the filtering configuration:
the filter is positioned between the first plate and the third plate,
the spacing between the first plate and the third plate is regulated by their spacers, the filter, and the deposited sample, and
the inner surface of the third plate presses the deposited sample against the filter, forcing at least one component of the sample to flow through the filter toward the first plate, thereby separating the at least one component from the sample; and
the closed configuration is configured after the third plate, the pressed sample, and the filter are removed from the first plate, and in the closed configuration:
the filtered at least one component left on the first plate is compressed by the first plate and the second plate into a layer of uniform thickness, and
the uniform thickness of the layer is confined by the inner surfaces of the first and second plates and is regulated by the plates and the spacers.

115. A method for sample analysis, comprising:
(a) obtaining a liquid sample that comprises one or more components,
(b) obtaining a device comprising a first plate, a second plate, a third plate, a filter and spacers, wherein:
i. the second plate and the third plate are respectively connected to the first plate, wherein the second plate and the third plate are configured to each pivot against the first plate without interfering with each other,
ii. by pivoting against the first plate, either the second plate or the third plate is movable relative to the first plate into different configurations,
iii. the first plate comprises an inner surface that has a sample contact area for contacting a liquid sample,
iv. the spacers are fixed on one or more of the plates or are mixed in the sample, and
v. the filter, having a sample receiving surface and a sample exit surface, is placed on top of the first plate with the sample exit surface facing the inner surface of the first plate,
(c) depositing a sample comprising one or more components on the sample receiving surface of the filter,
(d) pressing the third plate on the deposited sample against the filter, forcing at least one component of the sample to flow through the filter toward the first plate, thereby separating the at least one component from the sample,
(e) removing the third plate, the pressed sample, and the filter from the first plate, and
(f) compressing the filtered at least one component left on the first plate into a layer of uniform thickness by pressing the first plate and second plate together.

116. The device of claim 113, wherein the sample comprises a bodily fluid selected from the group consisting of: amniotic fluid, aqueous humour, vitreous humour, blood (e.g., whole blood, fractionated blood, plasma or serum), breast milk, cerebrospinal fluid (CSF), cerumen (earwax), chyle, chime, endolymph, perilymph, feces, breath, gastric acid, gastric juice, lymph, mucus (including nasal drainage and phlegm), pericardial fluid, peritoneal fluid, pleural fluid, pus, rheum, saliva, exhaled breath condensate, sebum, semen, sputum, sweat, synovial fluid, tears, vomit, urine, and a combination thereof.

117. The device of claim 113, wherein the sample is blood.

118. The device of claim 113, wherein the sample is an environmental sample from an environmental source selected from the group consisting of a river, lake, pond, ocean, glaciers, icebergs, rain, snow, sewage, reservoirs, tap water, drinking water, soil, compost, sand, rocks, concrete, wood, brick, sewage, the air, underwater heat vents, industrial exhaust, vehicular exhaust, and a combination thereof.

119. The device of claim 113, wherein the sample is a foodstuff sample selected from the group consisting of: raw ingredients, cooked food, plant and animal sources of food, preprocessed food, partially or fully processed food, and a combination thereof.

120. The device of claim 113, wherein the spacers have a filling factor of at least 1%, the filling factor being the ratio of the spacer area in the sample contact surface to the total area of the sample contact surface.

121. The device of claim 113, wherein the Young's modulus of the spacers times the filling factor of the spacers is equal or larger than 10 MPa, the filling factor being the ratio of the spacer area in the sample contact surface to the total area of the sample contact surface.

122. The device of claim 113, wherein the inter-spacer distance is in the range of 1 μm to 200 μm and the inter-spacer distance is substantially periodic.

123. The device of claim 113, wherein the inter-spacer distance is in the range of 7 μm to 200 μm and the sample is blood.

124. The device of claim 113, wherein the spacers have a density of at least 100/mm².

125. The device of claim 113, wherein the spacers have a density of at least 1000/mm².

126. The device of claim 113, wherein the spacers are pillars with a cross-sectional shape selected from round, polygonal, circular, square, rectangular, oval, elliptical, or any combination of the same.

127. The device of claim 113, wherein the average thickness of the layer of uniform thickness has a value equal to or less than 1 μm.

128. The device of claim 113, wherein the average thickness of the layer of uniform thickness has a value in the range of 1 μm to 10 μm.

129. The device of claim 113, wherein the average thickness of the layer of uniform thickness has a value in the range of 10 μm to 30 μm.

130. The device of claim 113, wherein the average thickness of the layer of uniform thickness has a value in the range of 2 μm to 3.8 μm and the sample is blood.

131. The device of claim 113, wherein the materials of the plate and the spacers are selected from polystyrene, PMMA, PC, COC, COP, or another plastic.

132. The device of claim 113, wherein the third plate is configured to press the sample against the filter when the third plate pivots toward the first plate.

133. The device of claim 113, wherein one edge of the second plate is connected to the inner surface of the first plate with a first hinge.

134. The device of claim 113, wherein one edge of the third plate is connected to the inner surface of the first plate with a second hinge.

135. The device of claim 113, wherein one edge of the second plate is connected to the inner surface of the first plate with a first hinge, and one edge of the third plate is connected to the inner surface of the first plate with a second hinge.

136. The device of claim 113, wherein in the closed configuration between the first plate and second plate, the third plate can be adjusted to pivot against the first plate and the second plate.

137. The device of claim 113, wherein the first plate comprises one or more notches on one or more of its edges, wherein the notches are positioned such that the second plate and/or the third plate are juxtaposed on the notches to facilitate the manipulation of pivoting of the second plate and the third plate.

138. The method of claim 115, wherein the sample comprises a bodily fluid selected from the group consisting of: amniotic fluid, aqueous humour, vitreous humour, blood (e.g., whole blood, fractionated blood, plasma or serum), breast milk, cerebrospinal fluid (CSF), cerumen (earwax), chyle, chime, endolymph, perilymph, feces, breath, gastric acid, gastric juice, lymph, mucus (including nasal drainage and phlegm), pericardial fluid, peritoneal fluid, pleural fluid, pus, rheum, saliva, exhaled breath condensate, sebum, semen, sputum, sweat, synovial fluid, tears, vomit, urine, and a combination thereof.

139. The method of claim 115, wherein the sample is blood.

140. The method of claim 115, wherein the sample is an environmental sample from an environmental source selected from the group consisting of a river, lake, pond, ocean, glaciers, icebergs, rain, snow, sewage, reservoirs, tap water, drinking water, soil, compost, sand, rocks, concrete, wood, brick, sewage, the air, underwater heat vents, industrial exhaust, vehicular exhaust, and a combination thereof.

141. The method of claim 115, wherein the sample is a foodstuff sample selected from the group consisting of: raw ingredients, cooked food, plant and animal sources of food, preprocessed food, partially or fully processed food, and a combination thereof.

142. The method of claim 115, wherein the spacers have a filling factor of at least 1%, the filling factor being the ratio of the spacer area in the sample contact surface to the total area of the sample contact surface.

143. The method of claim 115, wherein the Young's modulus of the spacers times the filling factor of the spacers is equal or larger than 10 MPa, the filling factor being the ratio of the spacer area in the sample contact surface to the total area of the sample contact surface.

144. The method of claim 115, wherein the inter-spacer distance is in the range of 1 μm to 200 μm and the inter-spacer distance is substantially periodic.

145. The method of claim 115, wherein the inter-spacer distance is in the range of 7 μm to 200 μm and the sample is blood.

146. The method of claim 115, wherein the spacers have a density of at least 100/mm².

147. The method of claim 115, wherein the spacers have a density of at least 1000/mm².

148. The method of claim 115, wherein the spacers are pillars with a cross-sectional shape selected from round, polygonal, circular, square, rectangular, oval, elliptical, or any combination of the same.

149. The method of claim 115, wherein the average thickness of the layer of uniform thickness has a value equal to or less than 1 μm.

150. The method of claim 115, wherein the average thickness of the layer of uniform thickness has a value in the range of 1 μm to 10 μm.

151. The method of claim 115, wherein the average thickness of the layer of uniform thickness has a value in the range of 10 μm to 30 μm.

152. The method of claim 115, wherein the average thickness of the layer of uniform thickness has a value in the range of 2 μm to 3.8 μm and the sample is blood.

153. The method of claim 115, wherein the materials of the plate and the spacers are selected from polystyrene, PMMA, PC, COC, COP, or another plastic.

154. The method of claim 115, wherein the third plate is configured to press the sample against the filter when the third plate pivots toward the first plate.

155. The method of claim 115, wherein one edge of the second plate is connected to the inner surface of the first plate with a first hinge.

156. The method of claim 115, wherein one edge of the third plate is connected to the inner surface of the first plate with a second hinge.

157. The method of claim 115, wherein one edge of the second plate is connected to the inner surface of the first plate with a first hinge, and one edge of the third plate is connected to the inner surface of the first plate with a second hinge.

158. The method of claim 115, wherein in the closed configuration between the first plate and second plate, the third plate can be adjusted to pivot against the first plate and the second plate.

159. The method of claim 115, wherein the first plate comprises one or more notches on one or more of its edges, wherein the notches are positioned such that the second plate and/or the third plate are juxtaposed on the notches to facilitate the manipulation of pivoting of the second plate and the third plate.
